SEQUENCE LISTING (20 sequences)

SEQ ID NO: 1 - protein, 170 aa, Oplophorus gracilirostris
MFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 2 - protein, 147 aa, Oplophorus gracilirostris
MFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPD

SEQ ID NO: 3 - protein, 10 aa, Oplophorus gracilirostris
GSLLFRVTIN

SEQ ID NO: 4 - protein, 13 aa, Oplophorus gracilirostris
GVTGWRLCENILA

SEQ ID NO: 5 - protein, 171 aa, Artificial sequence
  Synthetic peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 6 - protein, 148 aa, Artificial sequence
  Synthetic peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPD

SEQ ID NO: 7 - protein, 11 aa, Artificial sequence
  Synthetic peptide
GSLLFRVTINV

SEQ ID NO: 8 - protein, 13 aa, Artificial sequence
  Synthetic peptide
GVTGWRLCERILA

SEQ ID NO: 9 - protein, 158 aa, Artificial sequence
  Synthetic peptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTIN

SEQ ID NO: 10 - protein, 11 aa, Artificial sequence
  Synthetic peptide
VTGYRLFEEIL

SEQ ID NO: 11 - protein, 11 aa, Artificial sequence
  Synthetic peptide
VSGWRLFKKIS

SEQ ID NO: 12 - protein, 155 aa, Artificial sequence [sequence omitted]

SEQ ID NO: 13 - protein, 11 aa, Artificial sequence
  Synthetic peptide
GSMLFRVTINS

SEQ ID NO: 14 - protein, 22 aa, Artificial sequence
  Synthetic peptide
GSMLFRVTINSVSGWRLFKKIS

SEQ ID NO: 15 - protein, 5 aa, Artificial sequence
  Synthetic peptide
HHHHH

SEQ ID NO: 16 - protein, 6 aa, Artificial sequence
  Synthetic peptide
HHHHHH

SEQ ID NO: 17 - protein, 10 aa, Artificial sequence
  Synthetic peptide
EQKLISEEDL

SEQ ID NO: 18 - protein, 8 aa, Artificial sequence
  Synthetic peptide
DYKDDDDK

SEQ ID NO: 19 - protein, 8 aa, Artificial sequence
  Synthetic peptide
WSHPQFEK

SEQ ID NO: 20 - protein, 9 aa, Artificial sequence
  Synthetic peptide
YPYDVPDYA

(19) United States
(12) Patent Application Publication
(10) Pub. No.: US 2020/0200765 A1
(43) Pub. Date: Jun. 25, 2020

(54) BROAD SPECTRUM GPCR BINDING AGENTS

(71) Applicant: Promega Corporation, Madison, WI (US)

(72) Inventors: Sergiy Levin, Madison, WI (US); Michelle Boursier, Madison, WI (US); Rachel Friedman Ohana, Madison, WI (US); Thomas Kirkland, Madison, WI (US); Keith Wood, Madison, WI (US)

(21) Appl. No.: 16/703,534

(22) Filed: Dec. 4, 2019

Related U.S. Application Data
(60) Provisional application No. 62/775,207, filed on Dec. 4, 2018.

(51) Int. Cl. G01N 33/68; G01N 33/58; C07D 403/04; C07D 417/04; C07D 413/04; C07D 471/06; C07D 495/06; C07C 291/00; C07C 233/83; G01N 21/64; G01N 24/08

(52) U.S. Cl. CPC G01N 33/6848; G01N 33/582; C07D 403/04; C07D 417/04; C07D 413/04; C07D 471/06; G01N 2021/6439; C07C 291/00; C07C 233/83; G01N 21/6428; G01N 24/088; G01N 2560/00; C07D 495/06

(57) ABSTRACT

Provided herein are broad-spectrum G-Protein coupled receptor (GPCR) binding agents, detectable/isolatable compounds comprising such binding agents (e.g., broad-spectrum GPCR binding agents linked to a functional element and/or solid surface), and methods of use thereof for the detection/isolation of GPCRs.

### FIELD

Provided herein are broad-spectrum G-Protein coupled receptor (GPCR) binding agents, detectable/isolatable compounds comprising such binding agents (e.g., broad-spectrum GPCR binding agents linked to a functional element and/or solid surface), and methods of use thereof for the detection/isolation of GPCRs.

### BACKGROUND

G-Protein coupled receptors (GPCRs) are an important class of trans-membrane proteins. Due to their involvement in multiple diseases, they are targeted by many modern medicines and are also heavily researched for the development of new ones. Therefore, tools that allow interrogation GPCR-ligand interactions in live cells are needed.

### SUMMARY

Provided herein are broad-spectrum G-Protein coupled receptor (GPCR) binding agents, detectable/isolatable compounds comprising such binding agents (e.g., broad-spectrum GPCR binding agents linked to a functional element and/or solid surface), and methods of use thereof for the detection/isolation of GPCRs.

In some embodiments, provided herein are compositions comprising a broad-spectrum G-protein coupled receptor (GPCR) binding agent attached to a functional element or solid surface, wherein the broad-spectrum GPCR binding agent comprises:

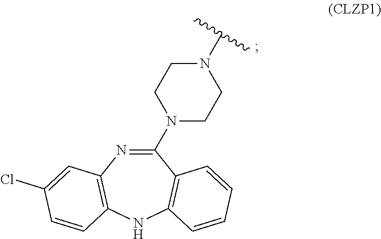

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

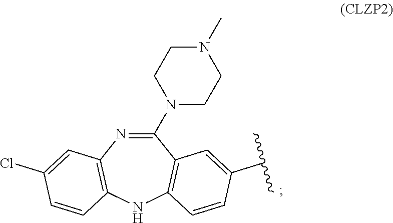

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

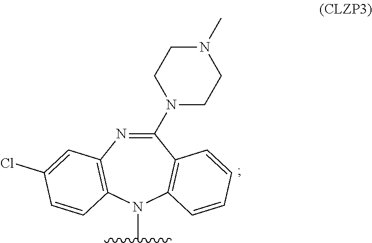

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

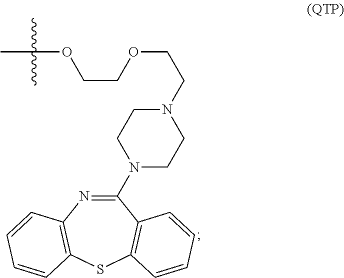

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

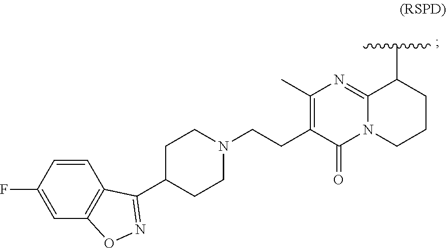

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

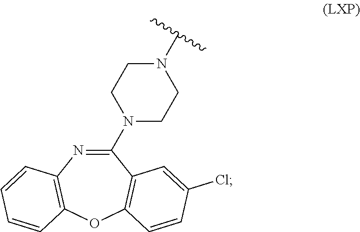

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

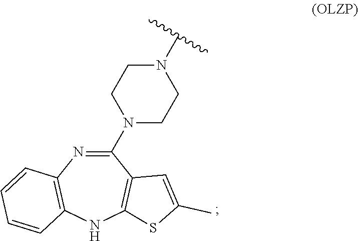

wherein [symbol] is the point of attachment of the broad-spectrum GPCR binding agent to the functional element, solid surface, or a linker between the broad-spectrum GPCR binding agent and the functional element or solid surface.

In some embodiments, provided herein are compositions comprising a broad-spectrum G-protein coupled receptor (GPCR) binding agent attached to a functional element or solid surface, wherein the broad-spectrum GPCR binding agent comprises:

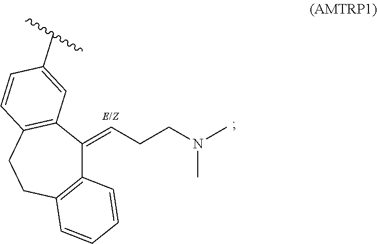

wherein [symbol] is the point of attachment of the broad-spectrum GPCR binding agent to the functional element, solid surface, or a linker between the broad-spectrum GPCR binding agent and the functional element or solid surface, and wherein the double bond may exist as the cis isomer (Z), trans isomer (E), or a mixture of the two.

In some embodiments, provided herein are compositions comprising a broad-spectrum G-protein coupled receptor (GPCR) binding agent attached to a functional element or solid surface, wherein the broad-spectrum GPCR binding agent comprises:

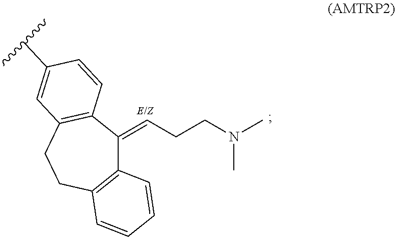

wherein [symbol] is the point of attachment of the broad-spectrum GPCR binding agent to the functional element, solid surface, or a linker between the broad-spectrum GPCR binding agent and the functional element or solid surface, and wherein the double bond may exist as the cis isomer (Z), trans isomer (E), or a mixture of the two.

In some embodiments, provided herein are compositions comprising a broad-spectrum G-protein coupled receptor (GPCR) binding agent attached to a functional element or solid surface, wherein the broad-spectrum GPCR binding agent comprises:

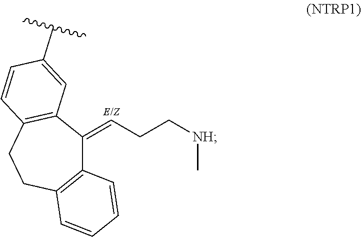

wherein [symbol] is the point of attachment of the broad-spectrum GPCR binding agent to the functional element, solid surface, or a linker between the broad-spectrum GPCR binding agent and the functional element or solid surface, and wherein the double bond may exist as the cis isomer (Z), trans isomer (E), or a mixture of the two.

In some embodiments, provided herein are compositions comprising a broad-spectrum G-protein coupled receptor (GPCR) binding agent attached to a functional element or solid surface, wherein the broad-spectrum GPCR binding agent comprises:

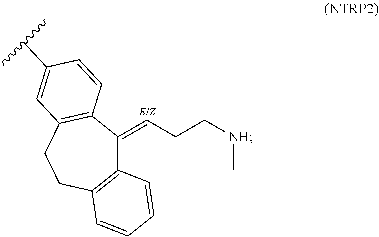

wherein [symbol] is the point of attachment of the broad-spectrum GPCR binding agent to the functional element, solid surface, or a linker between the broad-spectrum GPCR binding agent and the functional element or solid surface, and wherein the double bond may exist as the cis isomer (Z), trans isomer (E), or a mixture of the two.

In some embodiments, provided herein are compositions described herein (e.g., CLZP1, CLZP2, CLZP3, QTP, RSPD, LXP, OLZP, AMTRP1, AMTRP2, NTRP1, NTRP2, NTRP2, etc.) comprising a solid surface selected from a sedimental particle, a membrane, glass, a tube, a well, a self-assembled monolayer, a surface plasmon resonance chip, or a solid support with an electron conducting surface. In some embodiments, the sedimental particle is a magnetic particle.

In some embodiments, provided herein are compositions described herein (e.g., CLZP1, CLZP2, CLZP3, QTP, RSPD, LXP, OLZP, AMTRP1, AMTRP2, NTRP1, NTRP2, NTRP2, etc.) comprising a functional element selected from a detectable element, an affinity element, and a capture element. In some embodiments, the detectable element comprises a fluorophore, chromophore, radionuclide, electron opaque molecule, a MM contrast agent, SPECT contrast agent, or mass tag.

In some embodiments, the broad-spectrum GPCR binding agent of the compositions described herein (e.g., CLZP1, CLZP2, CLZP3, QTP, RSPD, LXP, OLZP, AMTRP1, AMTRP2, NTRP1, NTRP2, NTRP2, etc.) is attached to the functional element or solid surface directly. In some embodiments, the broad-spectrum GPCR binding agent of the compositions described herein (e.g., CLZP1, CLZP2, CLZP3, QTP, RSPD, LXP, OLZP, AMTRP1, AMTRP2, NTRP1, NTRP2, NTRP2, etc.) is attached to the functional element or solid surface via a linker. In some embodiments, the linker comprises [(CH₂)₂O]ₙ, wherein n is 1-20. In some embodiments, the linker is attached to the broad-spectrum GPCR binding agent and/or the functional element by an amide bond.

In some embodiments, provided herein are compositions comprising a structure of:

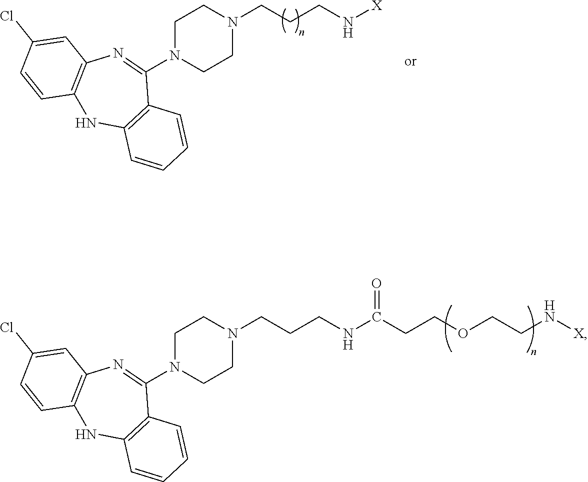

wherein n is 0-8, and wherein X is a functional element or solid surface.

In some embodiments, provided herein are compositions comprising a structure of:

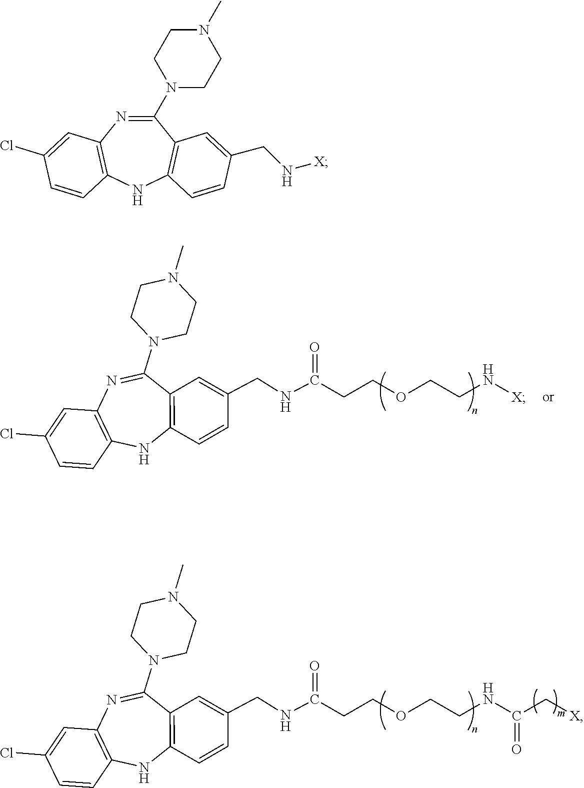

wherein n is 0-8, wherein m is 0-8, and wherein X is a functional element or solid surface.

In some embodiments, provided herein are compositions comprising a structure of:

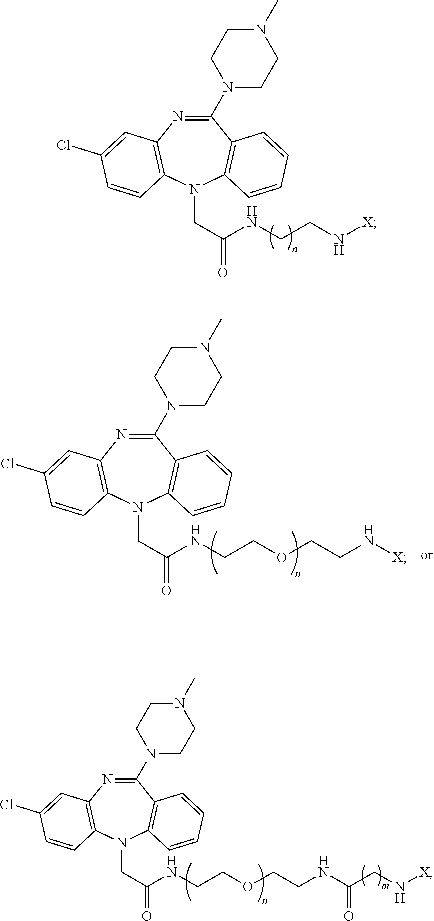

wherein n is 0-8, wherein m is 0-8, and wherein X is a functional element or solid surface.

In some embodiments, provided herein are compositions comprising a structure of:

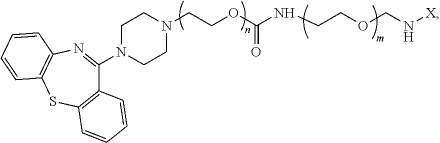

wherein n is 0-8, wherein m is 0-8, and wherein X is a functional element or solid surface.

In some embodiments, provided herein are compositions comprising a structure of:

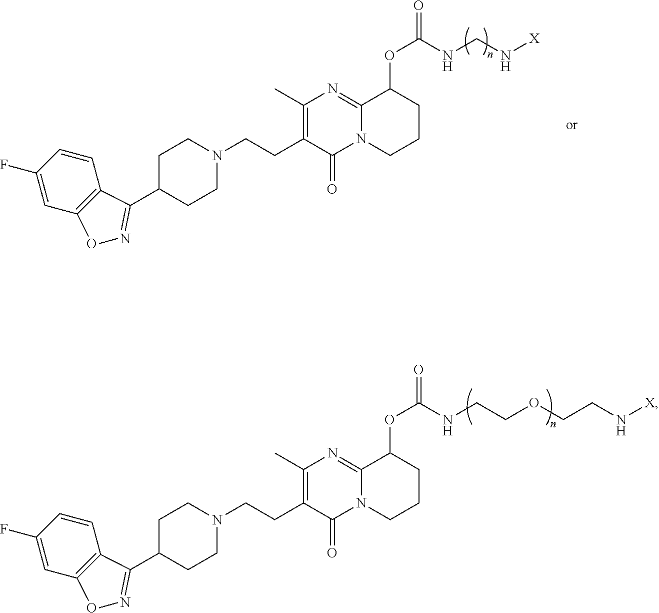

wherein n is 0-8, and wherein X is a functional element or solid surface.

In some embodiments, provided herein are compositions comprising a structure of:

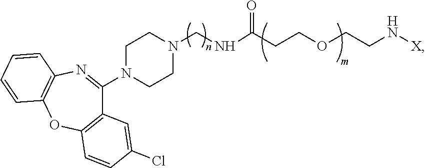

wherein n is 0-8, wherein m is 0-8, and wherein X is a functional element or solid surface.

In some embodiments, provided herein are compositions comprising a structure of:

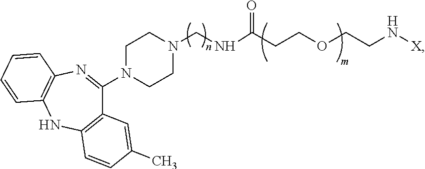

wherein n is 0-8, wherein m is 0-8, and wherein X is a functional element or solid surface.

In some embodiments, provided herein are compositions comprising a structure of:

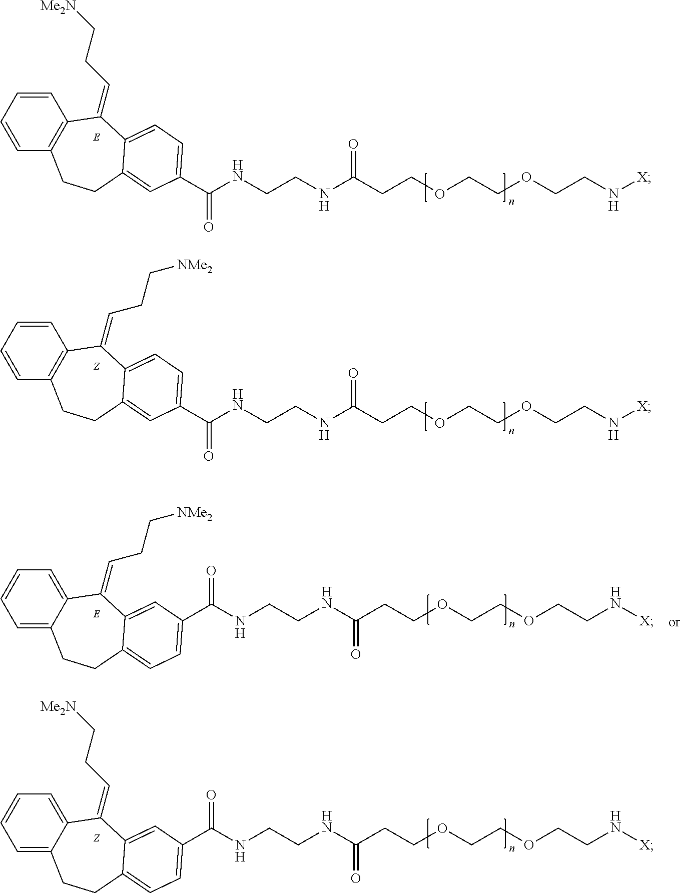

wherein n is 0-8, wherein X is a functional element or solid surface, and wherein any geometric isomers (e.g., C═C) may exist as the cis isomer (Z), trans isomer (E), or a mixture of the two.

In some embodiments, provided herein are compositions comprising a structure of:

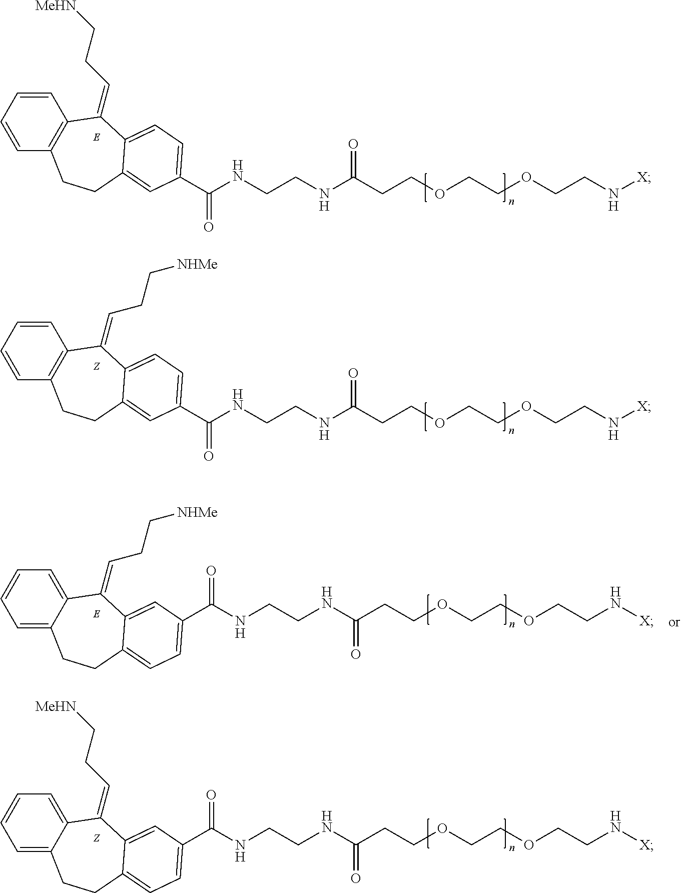

wherein n is 0-8 and wherein X is a functional element or solid surface.

In some embodiments, provided herein are compositions comprising a structure of:

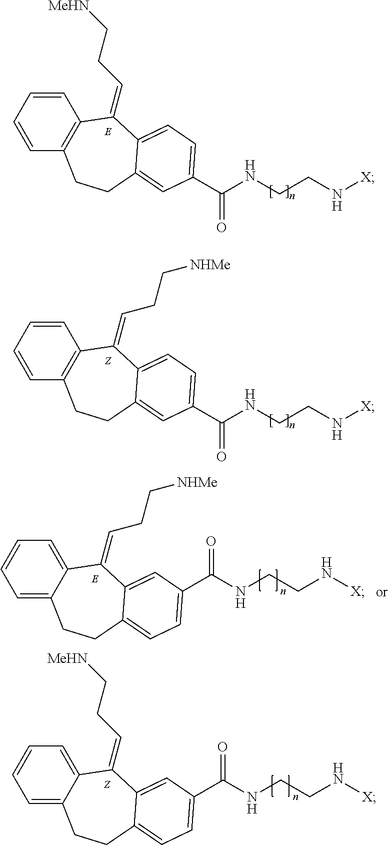

wherein n is 0-8 and wherein X is a functional element or solid surface.

In some embodiments, provided herein are compositions comprising a structure of:

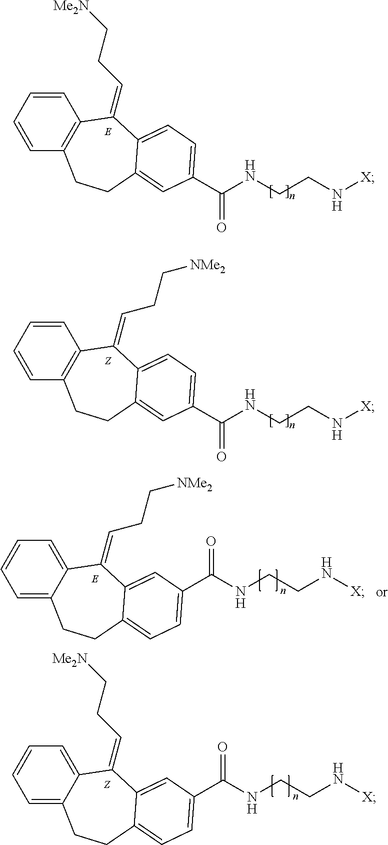

wherein n is 0-8 and wherein X is a functional element or solid surface.

In some embodiments, provided herein are compositions comprising a functional element (X) is a fluorophore.

In some embodiments, provided herein are compositions comprising a amitriptyline-based structure as depicted in FIG. 15 or a nortriptyline-based version of one of the structures depicted in FIG. 15. In some embodiments, a amitriptyline-based structure as depicted in FIG. 15 or a nortriptyline-based version thereof is provided with an alternative linker, fluorophore (or other X group), or connection point on the amitriptyline or nortriptyline ring system. In some embodiments, alternative linkers and X groups are provided herein and alternative connection points are provided by, for example, AMTRP1, AMTRP2, NTRP1, NTRP2, and NTRP2.

In some embodiments, a composition herein comprises a non-natural abundance of one or more stable heavy isotopes.

In some embodiments, provided herein are methods of detecting or quantifying GPCRs in a sample, comprising contacting the sample with a composition described herein (e.g., a composition comprising a linked GPCR binding agent and functional group or solid surface) and detecting or quantifying the functional element of a signal produced thereby. In some embodiments, the functional element of a signal produced thereby is detected or quantified by fluorescence, mass spectrometry, optical imaging, magnetic resonance imaging (MRI), and energy transfer.

In some embodiments, provided herein are methods of isolating GPCRs from a sample, comprising contacting the sample with a composition described herein (e.g., a composition comprising a linked GPCR binding agent and functional group or solid surface) and separating the functional element or the solid surface, as well as the bound GPCRs, from the unbound portion of the sample. In some embodiments, characterizing the identities of the GPCRs in a sample comprises isolating the GPCRs from a sample and analyzing the isolated GPCRs by mass spectrometry.

In some embodiments, provided herein are methods of monitoring interactions between GPCRs and unmodified biomolecules comprising contacting the sample with a composition described herein (e.g., a composition comprising a linked GPCR binding agent and functional group or solid surface).

In some embodiments, any of the methods described herein are performed using a sample selected from a cell, cell lysate, body fluid, tissue, biological sample, in vitro sample, and environmental sample.

In some embodiments, provided herein are systems comprising a composition described herein (e.g., a composition comprising a linked GPCR binding agent and functional group), wherein the functional element is a fluorophore; and (b) a fusion of a GPCR and a bioluminescent protein or a peptide component of a bioluminescent complex, wherein the emission spectrum of the bioluminescent protein or the bioluminescent complex overlaps the excitation spectrum of the fluorophore. In some embodiments, the system comprises a kit, cell, cell lysate, or reaction mixture. In some embodiments, the fusion comprises a GPCR and a peptide component of a bioluminescent complex, and wherein the system further comprises one or more additional components of the bioluminescent complex (e.g., a polypeptide component of the bioluminescent complex) and a substrate for the bioluminescent complex.

In some embodiments, provided herein are methods comprising: (a) contacting a fusion of a GPCR and a bioluminescent protein with (i) a composition described herein (e.g., a composition comprising a linked GPCR binding agent and functional group), wherein the functional element is a fluorophore, and wherein the emission spectrum of the bioluminescent protein overlaps the excitation spectrum of the fluorophore, and (ii) a substrate for the bioluminescent protein; and (b) detecting a wavelength of light within the excitation spectrum of the fluorophore resulting from bioluminescence resonance energy transfer from the bioluminescent protein to the fluorophore when the broad-spectrum GPCR binding agent is bound to the GPCR.

In some embodiments, provided herein are methods comprising: (a) contacting a fusion of a GPCR and a peptide component of a bioluminescent complex with (i) a composition described herein (e.g., a composition comprising a linked GPCR binding agent and functional group), wherein the functional element is a fluorophore, and wherein the emission spectrum of the bioluminescent protein overlaps the excitation spectrum of the fluorophore, (ii) a polypeptide component of the bioluminescent complex, and (iii) a substrate for the bioluminescent protein; and (b) detecting a wavelength of light within the excitation spectrum of the fluorophore resulting from bioluminescence resonance energy transfer from the bioluminescent complex to the fluorophore when the broad-spectrum GPCR binding agent is bound to the GPCR.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Schematic representation of a BRET experiment that utilizes displacement of a fluorescent ligand by a GPCR-ligand. Cells expressing HiBiT-GPCR fusion (left) are treated with LgBiT and fluorescent ligand. Upon formation of LgBiT-HiBiT complex and binding of fluorescent ligand to GPCR, a BRET signal appears (center). Treatment with non-labeled ligands displaces the fluorescent ligand resulting in the decrease of BRET signal (right).

FIG. 2. Schematic representation of discrimination of GPCR-HiBiT fusions from other non-fusion GPCRs by BRET signal.

FIG. 3A-I. Schemes for the chemical synthesis of exemplary clozapine-based tracers.

FIG. 4. Scheme for the chemical synthesis of exemplary loxapine-based tracers.

FIG. 5. Scheme for the chemical synthesis of exemplary olanzapine-based tracers.

FIG. 6. Scheme for the chemical synthesis of exemplary quetiapine-based tracers.

FIG. 7. Scheme for the chemical synthesis of exemplary risperidone-based tracers.

FIG. 8. Exemplary scheme for the detection of tracer binding to GPCR/HiBiT fusions via BRET.

FIG. 9. Heatmap of exemplary BRET results generated with fluorescently-tagged GPCR binding agent tracers against a diverse panel of GPCR/HiBiT fusions expressed in live cells. Assay signals were assessed by taking the ratio of BRET signals for tracer binding in the absence and presence of competing excess unmodified compound.

FIGS. 10A and 10B. BRET target engagement results in live cells with representative GPCR/HiBiT fusions. Cells transfected with plasmid DNA encoding each GPCR/HiBiT fusion were treated with serially diluted tracer (Clozapine tracer SL-1454 FIG. 10A) and (Respiredone tracer SL-1591 FIG. 10B) resulting in a dose-dependent increase of specific BRET.

FIG. 11. Structures of clozapine and its modifiable cores with exemplary attachment points marked on the unmodified clozapine structure.

FIG. 12. Structures of exemplary clozapine-based tracers.

FIG. 13. Structures of exemplary loxapine-, olanzapine-, quetiapine-, and risperidone-based tracers.

FIG. 14. Structures of exemplary linkers connecting a “drug” (GPCR binding agent) to a functional element.

FIG. 15A-B. Structures of exemplary (A) amitriptyline-based and (B) nortriptyline-based tracers. Alternative X groups (pink), such as other fluorophores, may be provided.

FIG. 16. Heat map of exemplary BRET results generated with fluorescently-tagged amitriptyline-derived GPCR binding agent against a diverse panel of GPCR/HiBiT fusions expressed in live cells. Assay signals were assessed by taking the ratio of BRET signals for tracer binding in the absence and presence of competing excess unmodified compound.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a GPCR” is a reference to one or more GPCRs and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “isomer” refers to compounds that have the same composition and molecular weight but differ in physical and/or chemical properties. The structural difference may be in constitution or in the ability to rotate the plane of polarized light.

As used herein, the terms “stereoisomers” or “geometric isomers” refer to the set of compounds which have the same number and type of atoms and share the same bond connectivity between those atoms, but differ in three-dimensional structure. The terms “stereoisomer” or “geometric isomer” refer to any member of this set of compounds.

As used herein, the term “clozapine” refers to a compound of the structure:

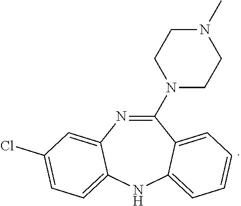

A clozapine moiety or substituent of a molecular entity comprises a clozapine structure tethered at any suitable point of attachment to another molecular entity (e.g., solid surface, functional element, etc.).

As used herein, the term “loxapine” refers to a compound of the structure:

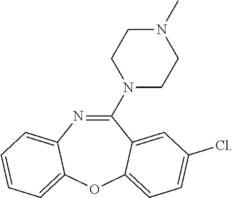

A loxapine moiety or substituent of a molecular entity comprises a loxapine structure tethered at any suitable point of attachment to another molecular entity (e.g., solid surface, functional element, etc.).

As used herein, the term “quetiapine” refers to a compound of the structure:

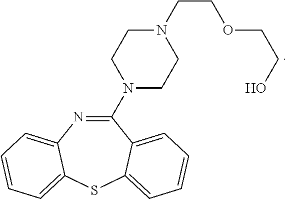

A quetiapine moiety or substituent of a molecular entity comprises a quetiapine structure tethered at any suitable point of attachment to another molecular entity (e.g., solid surface, functional element, etc.).

As used herein, the term “risperidone” refers to a compound of the structure:

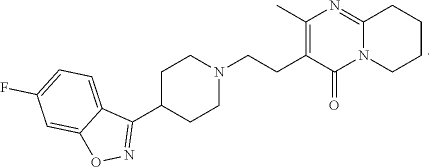

A risperidone moiety or substituent of a molecular entity comprises a risperidone structure tethered at any suitable point of attachment to another molecular entity (e.g., solid surface, functional element, etc.).

As used herein, the term “olanzapine” refers to a compound of the structure:

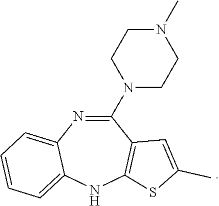

An olanzapine moiety or substituent of a molecular entity comprises an olanzapine structure tethered at any suitable point of attachment to another molecular entity (e.g., solid surface, functional element, etc.).

As used herein, the term “amitriptyline” refers to a compound of the structure:

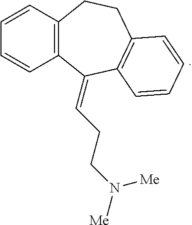

An amitriptyline moiety or substituent of a molecular entity comprises an amitriptyline structure tethered at any suitable point of attachment to another molecular entity (e.g., solid surface, functional element, etc.). Amitriptyline and amitriptyline moieties contain a C═C. Although amitriptyline is symmetrical any substitution that breaks the symmetry results in two geometric isomers of the double bond. Thus amitriptyline moieties may exist as the cis isomer (Z), trans isomer (E), or a mixture of the two.

As used herein, the term “nortriptyline” refers to a compound of the structure:

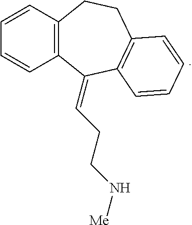

An nortriptyline moiety or substituent of a molecular entity comprises an nortriptyline structure tethered at any suitable point of attachment to another molecular entity (e.g., solid surface, functional element, etc.). Nortriptyline and nortriptyline moieties contain a C═C. Although nortriptyline is symmetrical any substitution that breaks the symmetry results in two geometric isomers of the double bond. Thus, nortriptyline moieties may exist as the cis isomer (Z), trans isomer (E), or a mixture of the two

As used herein, the term “tracer” refers to a compound of interest or an agent that binds to an analyte of interest (e.g., protein of interest (e.g., GPCR), etc.) and displays a quantifiable or detectable property (e.g., detected or quantified any suitable biochemical or biophysical technique (e.g., optically, magnetically, electrically, by resonance imaging, by mass, by radiation, etc.)). Tracers may comprise a compound of interest or an agent that binds to an analyte of interest linked (e.g., directly or via a suitable linker) to a fluorophore, radionuclide, mass tag, contrast agent for magnetic resonance imaging (MM), planar scintigraphy (PS), positron emission tomography (PET), single photon emission computed tomography (SPECT), and computed tomography (CT) (e.g., a metal ion chelator with bound metal ion, isotope, or radionuclide), etc.

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the term “linearly connected atoms” refers to the backbone atoms of a chain or polymer, excluding pendant, side chain, or H atoms that do not form the main chain or backbone.

As used herein, the term “functional element” refers to a detectable, reactive, affinity, or otherwise bioactive agent or moiety that is attached (e.g., directly or via a suitable linker) to a compound or moiety described herein. Other additional functional elements that may find use in embodiments described herein comprise “localization elements”, “detection elements”, etc.

As used herein, the term “capture element” refers to a molecular entity that forms a covalent interaction with a corresponding “capture agent”.

As used herein, the term “affinity element” refers to a molecular entity that forms a stable noncovalent interaction with a corresponding “affinity agent”.

As used herein, the term “solid support” is used in reference to any solid or stationary material to which reagents such as substrates, mutant proteins, drug-like molecules, and other test components are or may be attached. Examples of solid supports include microscope slides, wells of microtiter plates, coverslips, beads, particles, resin, cell culture flasks as well as many other suitable items. The beads, particles, or resin can be magnetic or paramagnetic.

As used herein in chemical structures, the indication:

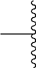

represents a point of attachment of one moiety to another moiety.

### DETAILED DESCRIPTION

Provided herein are broad-spectrum G-Protein coupled receptor (GPCR) binding agents, detectable/isolatable compounds comprising such binding agents (e.g., broad-spectrum GPCR binding agents linked to a functional element and/or solid surface), and methods of use thereof for the detection/isolation of GPCRs.

In some embodiments, provided herein are labeled GPCR ligands. Experiments were conducted during development of embodiments herein to demonstrate to select attachment point(s) on GPCR binding agents that produce a set of promiscuous tracers that retain binding profiles of the parent drug molecules, but include the additional functionality of the linked functional element, solid surface, etc. The labeled GPCR ligands described herein find use in any suitable assays.

In some embodiments, provided herein are compounds that bind a broad spectrum of GPCRs (e.g., specific to GPCRs, but not specific among GPCRs). In some embodiments, provided herein are compounds comprising a structure of one of:

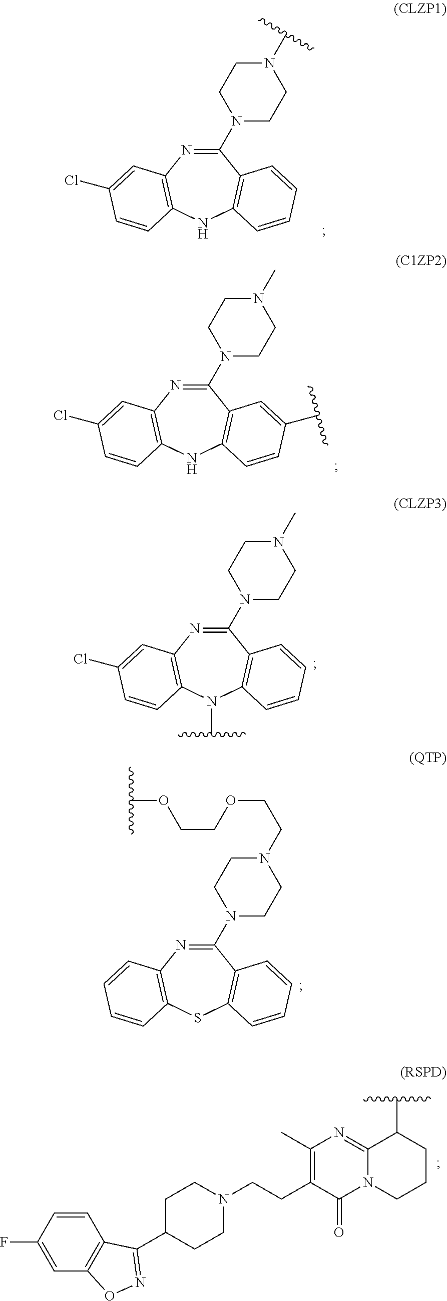

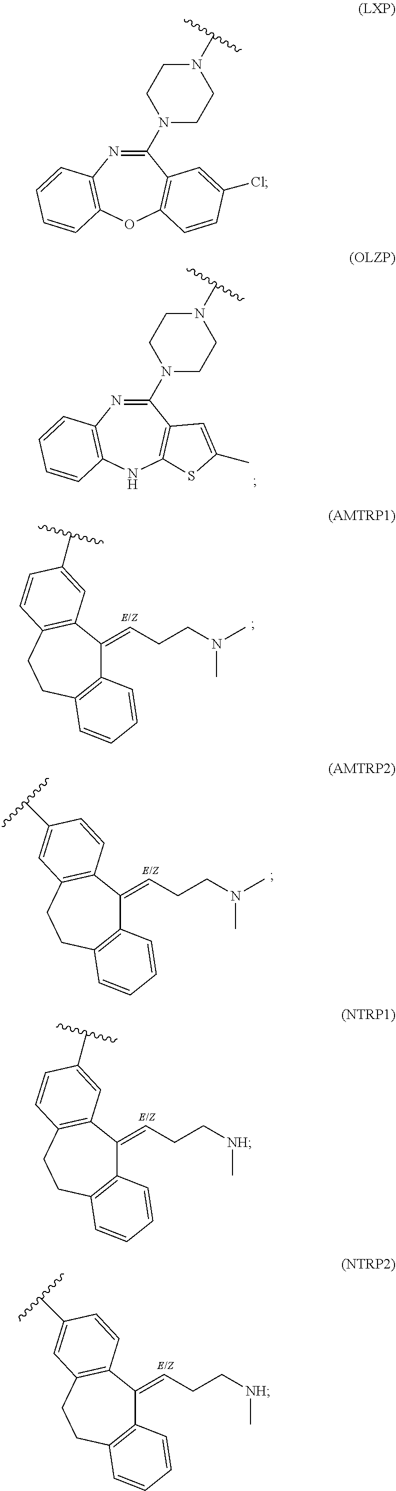

wherein [symbol] is the point of attachment of the broad-spectrum GPCR binding agent to a functional element, solid surface, or a linker between the broad-spectrum GPCR binding agent and the functional element or solid surface, and wherein geometric isomers may exist as the cis isomer (Z), trans isomer (E), or a mixture of the two.

In some embodiments, provided herein are analogs or derivatives of CLZP1, CLZP2, CLZP3, QTP, RSPD, LXP, OLZP, AMTRP1, AMTRP2, NTRP1, NTRP2. In some embodiments, CLZP1, CLZP2, CLZP3, QTP, RSPD, LXP, OLZP, AMTRP1, AMTRP2, NTRP1, NTRP2, or an analog or derivative thereof is attached directly (via a single covalent bond) to a functional element or solid surface. In some embodiments, CLZP1, CLZP2, CLZP3, QTP, RSPD, LXP, OLZP, AMTRP1, AMTRP2, NTRP1, NTRP2, or an analog or derivative thereof is attached indirectly (via a linker) to a functional element or solid surface.

In some embodiments, provided herein are compounds herein (e.g., comprising CLZP1, CLZP2, CLZP3, QTP, RSPD, LXP, OLZP, AMTRP1, AMTRP2, NTRP1, NTRP2, analogs or derivatives thereof, etc.) wherein [symbol] is a reactive group suitable for chemical conjugation to a functional element (e.g., detectable element, linker, etc.) or solid surface. These reactive groups may be present on the GPCR binding agent or connected by a suitable linker. In some embodiments, the reactive group is configured to react specifically (e.g., via biorthogonal, or click chemistry) with a reactive partner that is present or has been introduced on the functional element or solid surface. An exemplary click reaction is copper catalyzed click where the compound bears an alkyne or an azide, and the functional element bears the complementary group (e.g., an azide or an alkyne). Mixing these two species together in the presence of an appropriate copper catalyst causes the compound to be covalently conjugated to the functional element through a triazole. Many other biorthogonal reactions have been reported (for example Patterson, D. M., et al. (2014). “Finding the Right (Bioorthogonal) Chemistry.” ACS Chemical Biology 9(3): 592-605; herein incorporated by reference in its entirety), and compounds (e.g., comprising CLZP1, CLZP2, CLZP3, QTP, RSPD, LXP, OLZP, AMTRP1, AMTRP2, NTRP1, NTRP2, analogs or derivatives thereof, etc.) and functional elements incorporating complementary reactive species are embodiments of the present invention.

In some embodiments, a linker provides sufficient distance between moieties in a compound or composition herein (e.g., between a broad spectrum GPCR binding agent and detectable element, solid surface, etc.) to allow each to function undisturbed (or minimally disturbed) by the linkage to the other. For example, linkers provide sufficient distance to allow a GPCR binding agent to bind a GPCR and detectable moiety to be detectable (e.g., without or with minimal interference between the two). In some embodiments, a linker separates a GPCR binding agent herein (e.g., comprising CLZP1, CLZP2, CLZP3, QTP, RSPD, LXP, OLZP, AMTRP1, AMTRP2, NTRP1, NTRP2, analogs or derivatives thereof, etc.) and a functional element (e.g., detectable element, solid surface, etc.) by 5 angstroms to 1000 angstroms, inclusive, in length. Suitable linkers separate a compound herein and a functional element by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, and any suitable ranges therein (e.g., 5-100 Å, 50-500 Å, 150-700 Å, etc.). In some embodiments, the linker separates a compound herein and a functional element by 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, etc.)).

In some embodiments, a linker comprises 1 or more (e.g., 1-20 (e.g., 1, 2, 3, 4, 5, 6, 7, 8, 9, 10, 11, 12, 13, 14, 15, 16, 17, 18, 19, 20, or any ranges therebetween) —(CH₂)₂O— (oxyethylene) groups (e.g., —(CH₂)₂O—(CH₂)₂O—(CH₂)₂O—(CH₂)₂O—, —(CH₂)₂O—(CH₂)₂O—(CH₂)₂O—(CH₂)₂O—CH₂)₂O—, —(CH₂)₂O—(CH₂)₂O—(CH₂)₂O—(CH₂)₂O—CH₂)₂O—(CH₂)₂O—, etc.). In some embodiments, the linker is —(CH₂)₂O—(CH₂)₂O—(CH₂)₂O—(CH₂)₂O—.

In some embodiments, a linker comprises two or more “linker moieties” (L¹, L², etc.). In some embodiments, a linker comprises a cleavable (e.g., enzymatically cleavable, chemically cleavable, etc.) moiety (Y) and 0, 1, 2, of more “linker moieties” (L¹, L², etc.). In some embodiments, linker moieties are straight or branched chains comprising any combination of alkyl, alkenyl, or alkynyl chains, and main-chain heteroatoms (e.g., O, S, N, P, etc.). In some embodiments, linker moieties comprise one or more backbone groups selected from of: —O—, —S—, —CH═CH—, ═C═, a carbon-carbon triple bond, C═O, NH, SH, OH, CN, etc. In some embodiments, a linker moiety comprises one or more substituents, pendants, side chains, etc., comprising any suitable organic functional groups (e.g., OH, NH2, CN, ═O, SH, halogen (e.g., Cl, Br, F, I), COOH, CH₃, etc.).

In particular embodiments, a linker moiety comprises an alkyl carbamate group (e.g., (CH₂)ₙOCONH, (CH₂)ₙNHCOO, etc.). In some embodiments, the alkyl carbamate is oriented such that the —NH end is oriented toward the GPCR binding agent, and the COO end is oriented toward the functional element or solid surface. In some embodiments, the alkyl carbamate is oriented such the —COO end is oriented toward the GPCR binding agent and the —NH end is oriented toward the functional element or solid surface. In some embodiments, a linker or linker moiety comprises a single alkyl carbamate group. In some embodiments, a linker or linker moiety comprises two or more alkyl carbamate groups (e.g., 2, 3, 4, 5, 6, 7, 8, etc.).

In some embodiments, a linker moiety comprises more than 1 linearly connected C, S, N, and/or O atoms. In some embodiments, a linker moiety comprises one or more alkyl carbamate groups. In some embodiments, a linker moiety comprises one or more alkyl groups (e.g., methyl, ethyl, propyl, butyl, pentyl, hexyl, etc.). In some embodiments, a linker moiety comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)). In some embodiments, a linker moiety is 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)) in length.

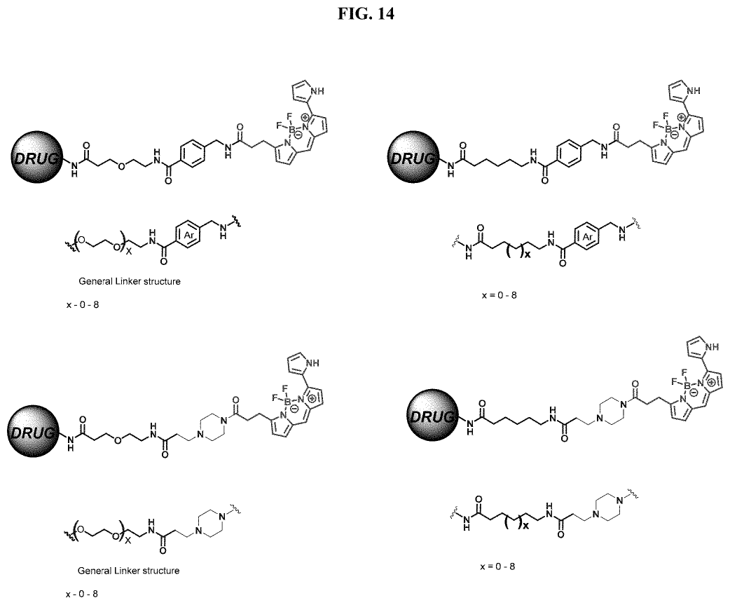

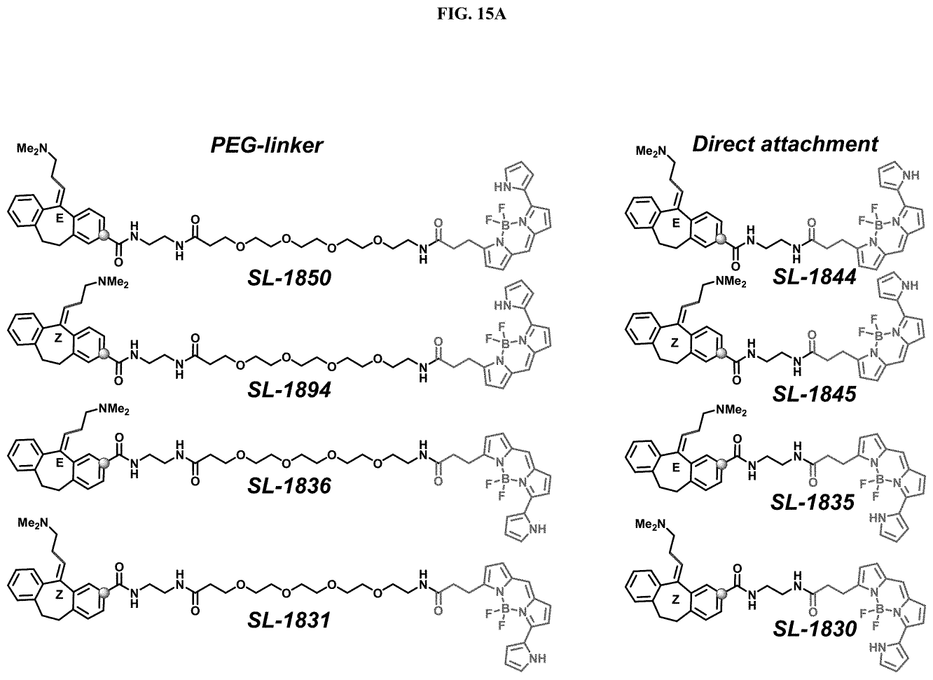

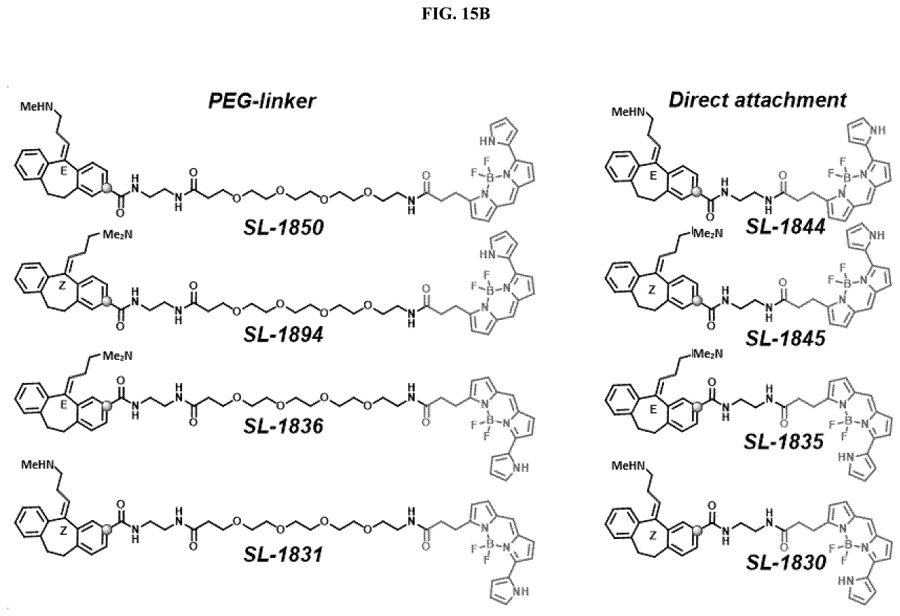

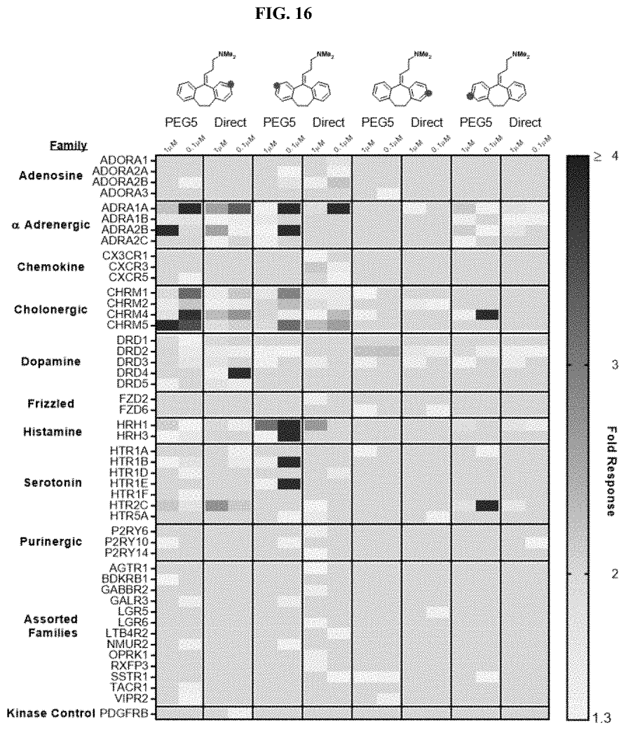

Exemplary linkers for connecting a “drug” (e.g., a GPCR binding agent herein (e.g., comprising CLZP1, CLZP2, CLZP3, QTP, RSPD, LXP, OLZP, AMTRP1, AMTRP2, NTRP1, NTRP2, analogs or derivatives thereof, etc.)) and a functional element (e.g., detectable element, solid surface, etc.) are depicted in FIG. 14. Such exemplary linkers find use with any suitable GPCR binding agents and functional elements described herein.

In some embodiments, the compositions described herein (e.g., comprising CLZP1, CLZP2, CLZP3, QTP, RSPD, LXP, OLZP, AMTRP1, AMTRP2, NTRP1, NTRP2, analogs or derivatives thereof, etc.) are biocompatible (e.g., cell compatible) and/or cell permeable. Therefore, in some embodiments, suitable functional elements (e.g., detectable, capture elements) are ones that are cell compatible and/or cell permeable within the context of such compositions. In some embodiments, a composition comprising an addition element, when added extracellularly, is capable of crossing the cell membrane to enter a cell (e.g., via diffusion, endocytosis, active transport, passive transport, etc.). In some embodiments, suitable functional elements and linkers are selected based on cell compatibility and/or cell permeability, in addition to their particular function.

In certain embodiments, functional elements have a detectable property that allows for detection of the compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) or an analyte (e.g., GPCR) bound thereto. Detectable functional elements include those with a characteristic electromagnetic spectral property such as emission or absorbance, magnetism, electron spin resonance, electrical capacitance, dielectric constant, or electrical conductivity as well as functional groups which are ferromagnetic, paramagnetic, diamagnetic, luminescent, electrochemiluminescent, fluorescent, phosphorescent, chromatic, antigenic, or have a distinctive mass. A functional element includes, but is not limited to, a nucleic acid molecule (e.g., DNA or RNA (e.g., an oligonucleotide or nucleotide), a protein (e.g., a luminescent protein, a peptide, a contrast agent (e.g., MRI contract agent), a radionuclide, an affinity tag (e.g., biotin or streptavidin), a hapten, an amino acid, a lipid, a lipid bilayer, a solid support, a fluorophore, a chromophore, a reporter molecule, a radionuclide, an electron opaque molecule, a MM contrast agent (e.g., manganese, gadolinium(III), or iron-oxide particles), or a coordinator thereof, and the like. Methods to detect a particular functional element, or to isolate a composition comprising a particular functional element and anything bound thereto, are understood.

In some embodiments, a functional group is or comprises a solid support. Suitable solid supports include a sedimental particle such as a magnetic particle, a sepharose, or cellulose bead; a membrane; glass, e.g., glass slides; cellulose, alginate, plastic, or other synthetically prepared polymer (e.g., an Eppendorf tube or a well of a multi-well plate); self-assembled monolayers; a surface plasmon resonance chip; or a solid support with an electron conducting surface; etc.

Exemplary detectable functional elements include haptens (e.g., molecules useful to enhance immunogenicity such as keyhole limpet hemacyanin), cleavable labels (e.g., photocleavable biotin) and fluorescent labels (e.g., N-hydroxysuccinimide (NETS) modified coumarin and succinimide or sulfonosuccinimide modified BODIPY (which can be detected by UV and/or visible excited fluorescence detection), rhodamine (R110, rhodols, CRG6, Texas Methyl Red (TAMRA), Rox5, FAM, or fluorescein), coumarin derivatives (e.g., 7 aminocoumarin, and 7-hydroxycoumarin, 2-amino-4-methoxynapthalene, 1-hydroxypyrene, resorufin, phenalenones or benzphenalenones (U.S. Pat. No. 4,812,409)), acridinones (U.S. Pat. No. 4,810,636), anthracenes, and derivatives of alpha and beta-naphthol, fluorinated xanthene derivatives including fluorinated fluoresceins and rhodols (e.g., U.S. Pat. No. 6,162,931), and bioluminescent molecules (e.g., luciferase (e.g., Oplophorus-derive luciferase (See e.g., U.S. application Ser. No. 12/773,002; U.S. application Ser. No. 13/287,986; herein incorporated by reference in their entireties) or GFP or GFP derivatives). A fluorescent (or bioluminescent) functional element may be used to sense changes in a system, like phosphorylation, in real-time. A fluorescent molecule, such as a chemosensor of metal ions may be employed to label proteins which bind the composition. A bioluminescent or fluorescent functional group such as BODIPY, rhodamine green, GFP, or infrared dyes finds use as a functional element and may, for instance, be employed in interaction studies (e.g., using BRET, FRET, LRET or electrophoresis).

Another class of functional elements includes molecules detectable using electromagnetic radiation and includes, but is not limited to, xanthene fluorophores, dansyl fluorophores, coumarins and coumarin derivatives, fluorescent acridinium moieties, benzopyrene based fluorophores, as well as 7-nitrobenz-2-oxa-1,3-diazole, and 3-N-(7-nitrobenz-2-oxa-1,3-diazol-4-yl)-2,3-diamino-propionic acid. Preferably, the fluorescent molecule has a high quantum yield of fluorescence at a wavelength different from native amino acids and more preferably has high quantum yield of fluorescence that can be excited in the visible, or in both the UV and visible, portion of the spectrum. Upon excitation at a preselected wavelength, the molecule is detectable at low concentrations either visually or using conventional fluorescence detection methods. Electrochemiluminescent molecules such as ruthenium chelates and its derivatives or nitroxide amino acids and their derivatives are detectable at femtomolar ranges and below.

In some embodiments, a functional element is a fluorophore. Suitable fluorophores for linking to the compounds herein (e.g., to form a fluorescent tracer) include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog) such as those described in U.S. patent application Ser. No. 13/682,589, herein incorporated by reference in its entirety.

In addition to fluorescent molecules, a variety of molecules with physical properties based on the interaction and response of the molecule to electromagnetic fields and radiation find use in the compositions and methods described herein. These properties include absorption in the UV, visible, and infrared regions of the electromagnetic spectrum, presence of chromophores that are Raman active and can be further enhanced by resonance Raman spectroscopy, electron spin resonance activity, and nuclear magnetic resonances and molecular mass, e.g., via a mass spectrometer.

In some embodiments, a functional element is a capture element. In some embodiments, a capture element is a substrate for a protein (e.g., enzyme), and the capture agent is that protein. In some embodiments, a capture element is a “covalent substrate” or one that forms a covalent bond with a protein or enzyme that it reacts with. The substrate may comprise a reactive group (e.g., a modified substrate) that forms a covalent bond with the enzyme upon interaction with the enzyme, or the enzyme may be a mutant version that is unable to reconcile a covalently bound intermediate with the substrate. In some embodiments, the substrate is recognized by a mutant protein (e.g., mutant dehalogenase), which forms a covalent bond thereto. In such embodiments, while the interaction of the substrate and a wild-type version of the protein (e.g., dehalogenase) results in a product and the regeneration of the wild-type protein, interaction of the substrate (e.g., haloalkane) with the mutant version of the protein (e.g., dehalogenase) results in stable bond formation (e.g., covalent bond formation) between the protein and substrate. The substrate may be any suitable substrate for any mutant protein that has been altered to form an ultra-stable or covalent bond with its substrate that would ordinarily only transiently bound by the protein. In some embodiments, the protein is a mutant hydrolase or dehalogenase. In some embodiments, the protein is a mutant dehalogenase and the substrate is a haloalkane. In some embodiments, the haloalkane comprises an alkane (e.g., C₂-C₂₀) capped by a terminal halogen (e.g., Cl, Br, F, I, etc.). In some embodiments, the haloalkane is of the formula A-X, wherein X is a halogen (e.g., Cl, Br, F, I, etc.), and wherein A is an alkane comprising 2-20 carbons. In certain embodiments, A comprises a straight-chain segment of 2-12 carbons. In certain embodiments, A is a straight-chain segment of 2-12 carbons. In some embodiments, the haloalkane may comprise any additional pendants or substitutions that do not interfere with interaction with the mutant dehalogenase.

In some embodiments, a capture agent is a SNAP-Tag, and a capture element is benzyl guanine (See, e.g., Crivat G, Taraska J W (January 2012). Trends in Biotechnology 30 (1): 8-16; herein incorporated by reference in its entirety). In some embodiments, a capture agent is a CLIP-Tag, and a capture element is benzyl cytosine (See, e.g., Gautier, et al. Chem Biol. 2008 February; 15(2):128-36; herein incorporated by reference in its entirety).

In some embodiments, a functional element is an affinity element (e.g., that binds to an affinity agent). Examples of such pairs would include: an antibody as the affinity agent and an antigen as the affinity element; a His-tag as the affinity element and a nickel column as the affinity agent; a protein and small molecule with high affinity as the affinity agent and affinity element, respectively (e.g., streptavidin and biotin), etc. Examples of affinity molecules include molecules such as immunogenic molecules (e.g., epitopes of proteins, peptides, carbohydrates, or lipids (e.g., any molecule which is useful to prepare antibodies specific for that molecule)); biotin, avidin, streptavidin, and derivatives thereof; metal binding molecules; and fragments and combinations of these molecules. Exemplary affinity molecules include His5 (HHHHH)(SEQ ID NO: 15), HisX6 (HHHHHH)(SEQ ID NO: 16), C-myc (EQKLISEEDL) (SEQ ID NO: 17), Flag (DYKDDDDK) (SEQ ID NO: 18), SteptTag (WSHPQFEK)(SEQ ID NO: 19), HA Tag (YPYDVPDYA) (SEQ ID NO: 20), thioredoxin, cellulose binding domain, chitin binding domain, S-peptide, T7 peptide, calmodulin binding peptide, C-end RNA tag, metal binding domains, metal binding reactive groups, amino acid reactive groups, inteins, biotin, streptavidin, and maltose binding protein. Another example of an affinity molecule is dansyllysine. Antibodies which interact with the dansyl ring are commercially available (Sigma Chemical; St. Louis, Mo.) or can be prepared using known protocols such as described in Antibodies: A Laboratory Manual (Harlow and Lane, 1988).

In some embodiments, provided herein are methods of using the compounds herein (e.g., comprising CLZP1, CLZP2, CLZP3, QTP, RSPD, LXP, OLZP, AMTRP1, AMTRP2, NTRP1, NTRP2, analogs or derivatives thereof, etc.) alone or attached to a functional element (e.g., directly of via a suitable linker) to detect, isolate, analyze, characterize, etc., GPCRs within a system (e.g., a cell, a cell lysate, a sample, a biochemical solution or mixture, a tissue, an organism, etc.).

In some embodiments, provided herein are methods of detecting one or more GPCRs in a sample, the method comprising contacting the sample with a compound herein (e.g., comprising CLZP1, CLZP2, CLZP3, QTP, RSPD, LXP, OLZP, AMTRP1, AMTRP2, NTRP1, NTRP2, analogs or derivatives thereof, etc.). In some embodiments, provided herein are methods to isolate one or more GPCRs from a sample.

In some embodiments, methods are provided for characterizing a sample by analyzing the presence, quantity, and or population of GPCRs in the sample (e.g., what GPCRs are present and/or at what quantities?) by contacting the sample with a compound herein (e.g., comprising CLZP1, CLZP2, CLZP3, QTP, RSPD, LXP, OLZP, AMTRP1, AMTRP2, NTRP1, NTRP2, analogs or derivatives thereof, etc.).

In some embodiments, provided herein are methods of diagnosing a disease of condition comprising detecting the presence or quantity of one or more GPCRs in a sample from the subject by contacting the sample with a compound herein (e.g., comprising CLZP1, CLZP2, CLZP3, QTP, RSPD, LXP, OLZP, AMTRP1, AMTRP2, NTRP1, NTRP2, analogs or derivatives thereof, etc.), wherein the presence or quantity of the one or more of the GPCRs in the sample is indicative of the disease, condition, or a predisposition thereto.

In some embodiments, provided herein are methods of monitoring a subject's response to a therapeutic treatment comprising: (a) detecting the presence or quantity of one or more GPCRs in a sample from the subject by contacting the sample with compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) prior to administration of the therapeutic treatment, and (b) detecting the presence or quantity of one or more GPCRs in a sample from the subject by contacting the sample with compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) following administration of the therapeutic treatment, wherein a change in the presence of quantity of the one or more GPCRs is indicative of the subject's response to the therapeutic treatment.

In some embodiments, GPCRs bound by the compounds herein are detected, quantified, and/or isolated by taking advantage of unique properties of the compound and/or the functional element bound thereto by any means including electrophoresis, gel filtration, high-pressure or fast-pressure liquid chromatography, mass spectroscopy, affinity chromatography, ion exchange chromatography, chemical extraction, magnetic bead separation, precipitation, hydrophobic interaction chromatography (HIC), or any combination thereof. The isolated GPCR(s) may be employed for structural and functional studies, for diagnostic applications, for the preparation biological or pharmaceutical reagents, as a tool for the development of drugs, and for studying protein interactions, for the isolation and characterization of protein complexes, etc.

In some embodiments, methods are provided for detecting and/or quantifying a compound herein (e.g., comprising CC CLZP1, CLZP2, CLZP3, QTP, RSPD, LXP, OLZP, AMTRP1, AMTRP2, NTRP1, NTRP2, analogs or derivatives thereof, etc.) and/or analyte (e.g., GPCRs) bound thereto in a sample. In some embodiments, techniques for detection and/or quantification of a compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) and/or analyte (e.g., GPCRs) bound thereto depend upon the identity of the functional element attached to the compound (e.g., capture element, affinity element, detectable element (e.g., fluorophore, luciferase, chelated radionuclide, chelated contrast agent, etc.) and/or specific modifications to the compound (e.g., mass tags (e.g., heavy isotopes (e.g., ¹³C, ¹⁵N, ²H, etc.). For example, when a compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) is linked to a fluorophore or other light emitting functional element, the compound and/or analyte (e.g., GPCRs) bound thereto may be detected/quantified in a sample using systems, devices, and/or apparatuses that are provided to detect, quantitate, or monitor, the amount of light (e.g., fluorescence) emitted or changes thereto. In some embodiments, detection, quantification, and/or monitoring are provided by a device, system, or apparatus comprising one or more of a spectrophotometer, fluorometer, luminometer, photomultiplier tube, photodiode, nephlometer, photon counter, electrodes, ammeter, voltmeter, capacitative sensors, flow cytometer, CCD, etc.

In addition to fluorescent functional elements, a variety of functional elements with physical properties based on the interaction and response of the functional elements to electromagnetic fields and radiation can be used to detect the compound herein (e.g., comprising CLZP1, CLZP2, CLZP3, QTP, RSPD, LXP, OLZP, AMTRP1, AMTRP2, NTRP1, NTRP2, analogs or derivatives thereof, etc.) and/or a bound GPCR. These properties include absorption in the UV, visible, and infrared regions of the electromagnetic spectrum, presence of chromophores that are Raman active and can be further enhanced by resonance Raman spectroscopy, electron spin resonance activity, nuclear magnetic resonances, and molecular mass, e.g., via a mass spectrometer.

In some embodiments, the compounds herein bind a broad spectrum of GPCRs, including protein GPCRs of Class A, Class B. Class C, Class Frizzled, Adhesion class, and other seven transmembrane proteins. In some embodiments, the binding agents herein bind to multiple different GPCRs and/or GPCRs of multiple GPCR families, such as 5-Hydroxytryptamine receptors, Acetylcholine receptors (muscarinic), Adenosine receptors, Adrenoceptors, Angiotensin receptors, Apelin receptor, Bile acid receptor, Bombesin receptors, Bradykinin receptors, Cannabinoid receptors, Chemerin receptors, Chemokine receptors, Cholecystokinin receptors, Class A Orphans, Complement peptide receptors, Dopamine receptors, Endothelin receptor, Formylpeptide receptors, Free fatty acid receptors, Galanin receptors, Ghrelin receptor, Glycoprotein hormone receptors, Gonadotrophin-releasing hormone receptors, Histamine receptors, Hydroxycarboxylic acid receptors, Leukotriene receptors, Lysophospholipid (LPA) receptors, Lysophospholipid (SIP) receptors, Melanin-concentrating hormone receptors, Melanocortin receptors, Melatonin receptors, Neuromedin U receptors, Neuropeptide FF/neuropeptide AF receptors, Neuropeptide W/neuropeptide B receptors, Neuropeptide Y receptors, Neurotensin receptors, Opioid receptors, Opsin receptors, Orexin receptors, P2Y receptors, Prokineticin receptors, Prolactin-releasing peptide receptor, Prostanoid receptors, Proteinase-activated receptors, QRFP receptor, Relaxin family peptide receptors, Somatostatin receptors, Succinate receptor, Tachykinin receptors, Thyrotropin-releasing hormone receptors, Trace amine receptor, Urotensin receptor, Vasopressin and oxytocin receptors, Callcitonin receptors, Corticotropin-releasing factor receptors, Glucagon receptor family, Parathyroid hormone receptors, VIP and PACAP receptors. Calcium-sensing receptor, Class C Orphans, GABA_{B} receptors, Metabotropic glutamate receptors, Taste 1 receptors, Class Frizzled GPCRs, Adhesion Class GPCRs, etc. In some embodiments, the compounds herein bind to GPCRs of any suitable organism. In some embodiments, compounds herein bind to human GPCRs and/or homologs and analogs from other organisms.

In some embodiments, the binding agents herein are broad-spectrum GPCR binding agents. Therefore, a binding agent herein may bind to GPCRs of multiple (e.g., 2, 3, 4, 5, 10, 20, 30, 40, or more) GPCR classes or families. In some embodiments, a binding agent herein binds multiple (e.g., 2, 3, 4, 5, 10, 20, 30, 40, 50, 75, 100, 150, 200, 250, 300, 400, 500, or more) distinct GPCRs.

In some embodiments, the GPCR binding agents and tracers described herein find use in systems further comprising bioluminescent proteins (or bioluminescent complexes), and method of using such systems to generate bioluminescent resonance energy transfer (BRET) for the detection, characterization, monitoring, etc., of GPCRs. As such, the present disclosure includes materials and methods related to bioluminescent polypeptides, bioluminescent complexes and components thereof, and bioluminescence resonance energy transfer (BRET).

In some embodiments, provided herein are assays, devices, methods, and systems incorporating bioluminescent polypeptides and/or bioluminescent complexes (of peptide(s) and/or polypeptide components) based on (e.g., structurally, functionally, etc.) the luciferase of Oplophorus gracihrostris, the NanoLuc luciferase (Promega Corporation; U.S. Pat. Nos. 8,557,970; 8,669,103; herein incorporated by reference in their entireties), and/or the NanoBiT (U.S. Pat. No. 9,797,889; herein incorporated by reference in its entirety), or NanoTrip (U.S. Prov. App. No. 62/684,014). As described below, in some embodiments, the assays, devices, methods, and systems herein incorporate commercially available NanoLuc-based technologies (e.g., NanoLuc luciferase, NanoBRET, NanoBiT, NanoTrip, NanoGlo, etc.), but in other embodiments, various combinations, variations, or derivations from the commercially available NanoLuc-based technologies are employed.

PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides. Such polypeptides find use in embodiments herein and can be used in conjunction with the assays and methods described herein.

In some embodiments, assays, methods, devices, and systems herein comprise a bioluminescent polypeptide of SEQ ID NO: 5, or having at least 60% (e.g., 06%, 65%, 70%, 75%, 80%, 85%, 90%, 95%, 96%, 97%, 98%, 99%, 100%, or ranges therebetween) sequence identity with SEQ ID NO: 5. In some embodiments, a bioluminescent polypeptide is fused to a GPCR or otherwise linked to a component of the assays, methods, devices, and/or systems described herein.

PCT Appln. No. PCT/US14/26354 and U.S. Pat. No. 9,797,889 (each of which is herein incorporated by reference in their entirety and for all purposes) describe compositions and methods for the assembly of bioluminescent complexes. Such complexes, and the peptide and polypeptide components thereof, find use in embodiments herein and can be used in conjunction with the assays, methods, devices, and/or systems described herein. In some embodiments, provided herein are polypeptides having at least 60% (e.g., 60%, 65%, 70%, 75%, 80%, 85%, 90%, 95%, 96%, 97%, 98%, 99%, 100%, or ranges therebetween) sequence identity with SEQ ID NO: 9, but less than 100% (e.g., <99%, <98%, <97%, <96%, <95%, <94%, <93%, <92%, <91%, <90%) sequence identity with SEQ ID NO: 1, SEQ ID NO: 2, SEQ ID NO: 5, and SEQ ID NO: 6. In some embodiments, provided herein are peptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 10, but less than 100% (e.g., <99%, <98%, <97%, <96%, <95%, <94%, <93%, <92%, <91%, <90%) sequence identity with SEQ ID NO: 1, SEQ ID NO: 4, SEQ ID NO: 5, and SEQ ID NO: 8. In some embodiments, provided herein are peptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 11, but less than 100% (e.g., <99%, <98%, <97%, <96%, <95%, <94%, <93%, <92%, <91%, <90%) sequence identity with SEQ ID NO: 1, SEQ ID NO: 4, SEQ ID NO: 5, and SEQ ID NO: 8. In some embodiments, any of the aforementioned NanoBiT-based peptides or polypeptides are fused to a GPCR or otherwise linked (e.g., fused, chemically linked, etc.) to a component of the assays, methods, devices, and/or systems described herein.

U.S. Prov. App. No. 62/684,014 (herein incorporated by reference in its entirety and for all purposes) describes compositions and methods for the assembly of bioluminescent complexes. Such complexes, and the peptides and polypeptide components thereof, find use in embodiments herein and can be used in conjunction with the assays and methods described herein. In some embodiments, provided herein are polypeptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 12, but less than 100% (e.g., <99%, <98%, <97%, <96%, <95%, <94%, <93%, <92%, <91%, <90%) sequence identity with SEQ ID NO: 1, SEQ ID NO: 2, SEQ ID NO: 5, SEQ ID NO: 6, and SEQ ID NO: 9. In some embodiments, provided herein are peptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 11, but less than 100% (e.g., <99%, <98%, <97%, <96%, <95%, <94%, <93%, <92%, <91%, <90%) sequence identity with SEQ ID NO: 1, SEQ ID NO: 4, SEQ ID NO: 5, and SEQ ID NO: 8. In some embodiments, provided herein are peptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 13, but less than 100% (e.g., <99%, <98%, <97%, <96%, <95%, <94%, <93%, <92%, <91%, <90%) sequence identity with SEQ ID NO: 1, SEQ ID NO: 3, SEQ ID NO: 5, and SEQ ID NO: 7. In some embodiments, provided herein are peptides having at least 60% (e.g., 06%, 65%, 70%, 75%, 80%, 85%, 90%, 95%, 96%, 97%, 98%, 99%, 100%, or ranges therebetween) sequence identity with SEQ ID NO: 14, but less than 100% (e.g., <99%, <98%, <97%, <96%, <95%, <94%, <93%, <92%, <91%, <90%) sequence identity with SEQ ID NO: 1, SEQ ID NO: 3, SEQ ID NO: 4, SEQ ID NO: 5, SEQ ID NO: 7, and SEQ ID NO: 8. In some embodiments, any of the aforementioned NanoTrip-based peptides or polypeptides are fused to a GPCR or otherwise linked (e.g., fused, chemically linked, etc.) to a component of the assays, methods, devices, and/or systems described herein.

PCT Appln. No. PCT/US13/74765 and U.S. patent application Ser. No. 15/263,416 (herein incorporated by reference in their entireties and for all purposes) describe bioluminescence resonance energy transfer (BRET) systems and methods (e.g., incorporating NanoLuc-based technologies). Such systems and methods, and the bioluminescent polypeptide and fluorophore-conjugated components thereof, find use in embodiments herein and can be used in conjunction with the assays, methods, devices, and systems described herein

In some embodiments, any NanoLuc-based, NanoBiT-based, and/or NanoTrip-based peptides, polypeptide, complexes, fusions, etc. may find use in BRET-based applications with the assays, methods, devices, and systems described herein.

As used herein, the term “energy acceptor” refers to any small molecule (e.g., chromophore), macromolecule (e.g., autofluorescent protein, phycobiliproteins, nanoparticle, surface, etc.), or molecular complex that produces a readily detectable signal in response to energy absorption (e.g., resonance energy transfer). In certain embodiments, an energy acceptor is a fluorophore or other detectable chromophore (e.g., any fluorophore or other detectable chromophore described herein or understood in the field). Suitable fluorophores include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog) such as those described in U.S. patent application Ser. No. 13/682,589, herein incorporated by reference in its entirety.

In some embodiments, systems are provided comprising: (a) a fusion of a GPCR and a bioluminescent protein (or a component of a bioluminescent complex); and (b) a broad spectrum GPCR binding moiety herein linked to a fluorophore, wherein the emission spectrum of the bioluminescent protein overlaps the excitation spectrum of the fluorophore such that BRET is detectable between the bioluminescent protein and the fluorophore when the broad spectrum GPCR binding moiety binds to the GPCR. Similar BRET systems (e.g., utilizing a NANOLUC luciferase) are described in, for example, Intl. Pat. App. PCT/US13/74765 (herein incorporated by reference in its entirety), embodiments of which will find use in the systems and methods herein.

U.S. Pat. Nos. 10,107,800; 9,869,670; and 9,797,890 (herein incorporated by reference in their entireties) describe binary systems for assembly of a bioluminescent complex from peptide and polypeptide components. U.S. Prov. App. No. 62/684,014 (herein incorporated by reference in its entirety) describes tripartite systems for assembly of a bioluminescent complex from three peptide and polypeptide components. In some embodiments, such systems (and the methods associated therewith) find use in embodiments herein. For example, a peptide component of a bioluminescent complex is provided as a fusion with one or more GPCRs. When such a fusion is contacted with a broad spectrum GPCR fluorescent tracer described herein and the polypeptide component of the bioluminescent complex, a BRET signal is detectable. However, non-target GPCRs that are not fused to a peptide component of the bioluminescent complex, despite being bound by the broad spectrum GPCR fluorescent tracer, will not produce a BRET signal. In some embodiments, a peptide tag (e.g., fused to a GPCR) and other components of a bioluminescent complex system (e.g., polypeptide component, substrate, etc.) are described in the above patents/applications and/or commercially available as NanoBiT and/or NanoTrip technologies (Promega Corp., Madison, Wis.). In some embodiments, a peptide tag that is fused to a GPCR for BRET applications exhibits high affinity for the polypeptide component of the bioluminescent complex (e.g., and/or additional peptide components), such that the bioluminescent complex forms upon introduction of the appropriate components without facilitation.

In some embodiments, BRET applications of the technologies described herein rely on minimally perturbing GPCR protein structure by genetic fusion to a peptide component of a bioluminescent complex. In some embodiments, the peptide exhibits high affinity of the polypeptide component and/or other peptide components of the bioluminescent complex (e.g., HiBiT). In some embodiments, the fusion is made at the N-terminus, C-terminus, and out internally within the GPCR. In some embodiments, the small size of the peptide tag allows for minimal genetic manipulation of the protein. Experiments conducted during development of embodiments herein have demonstrated that a peptide tag (e.g., a component of a bioluminescent complex (e.g., HiBiT)) is easily inserted into a GPCR of interest through CRISPR-Cas, thus allowing GPCR-ligand interaction interrogation at the endogenous level without the need to overexpress GPCRs and/or use of membrane preparations. In some embodiments, the polypeptide component of a bioluminescent complex (e.g., LgBiT) is not cell-permeable, thus a signal is only detected on the cell surface. This feature of the detection method allows monitoring of both GPCR cell surface expression levels and internalization.

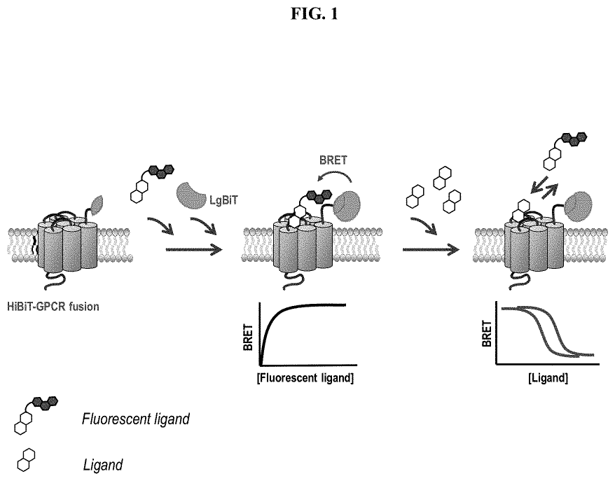

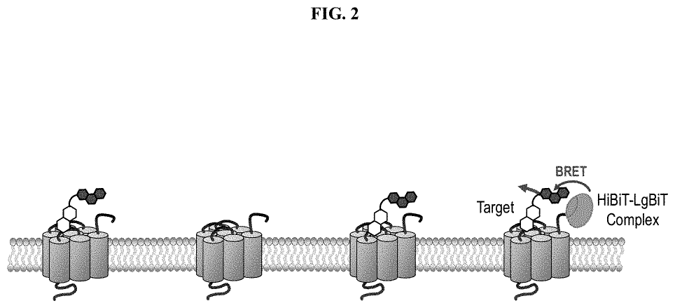

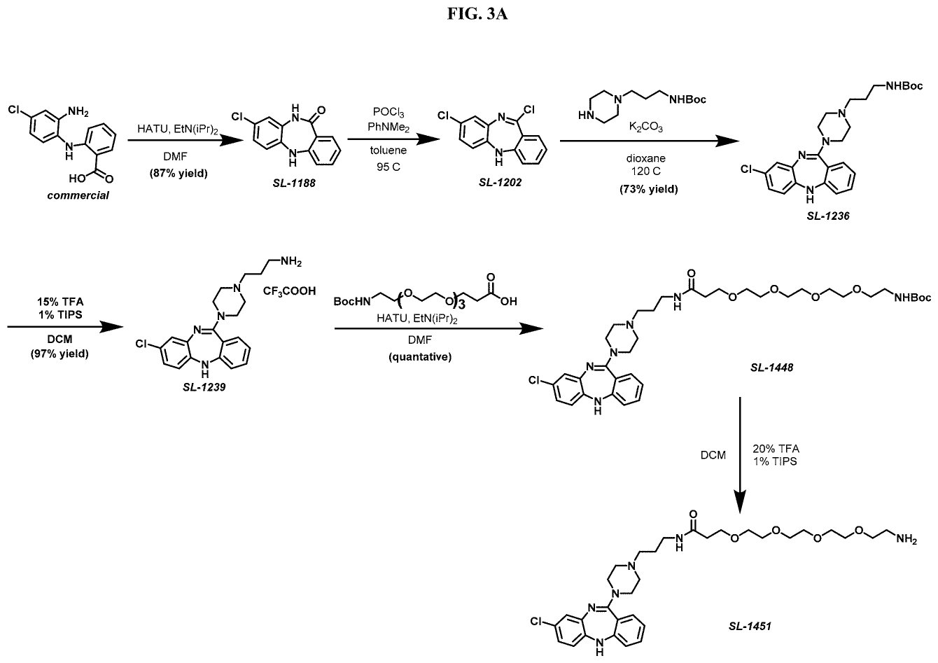

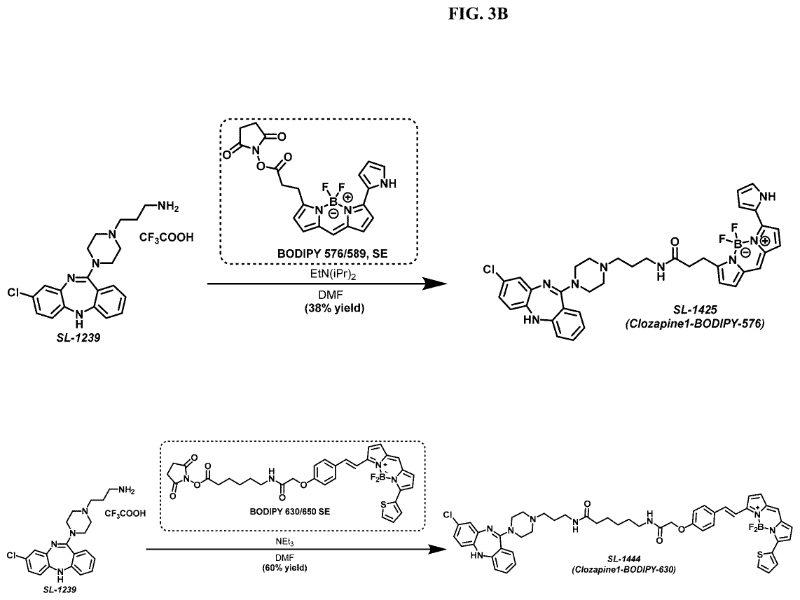

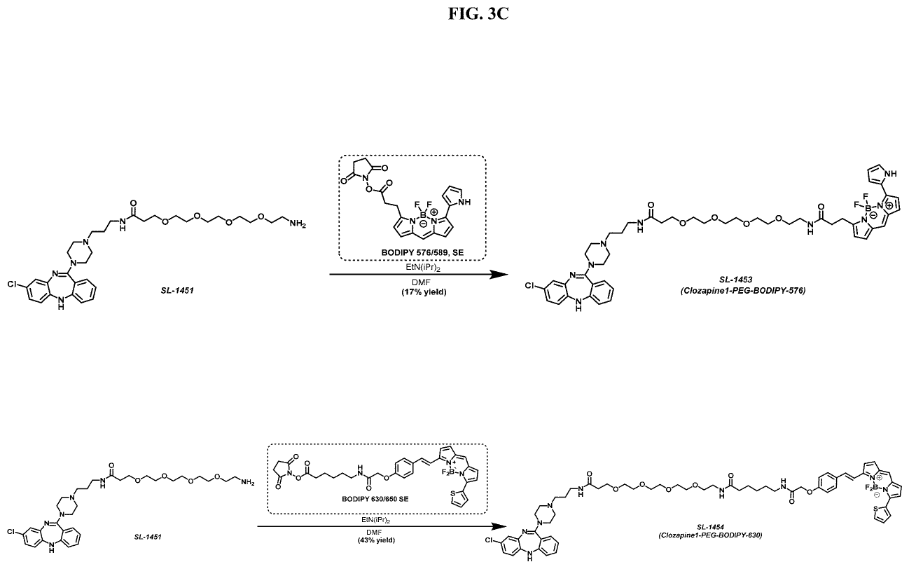

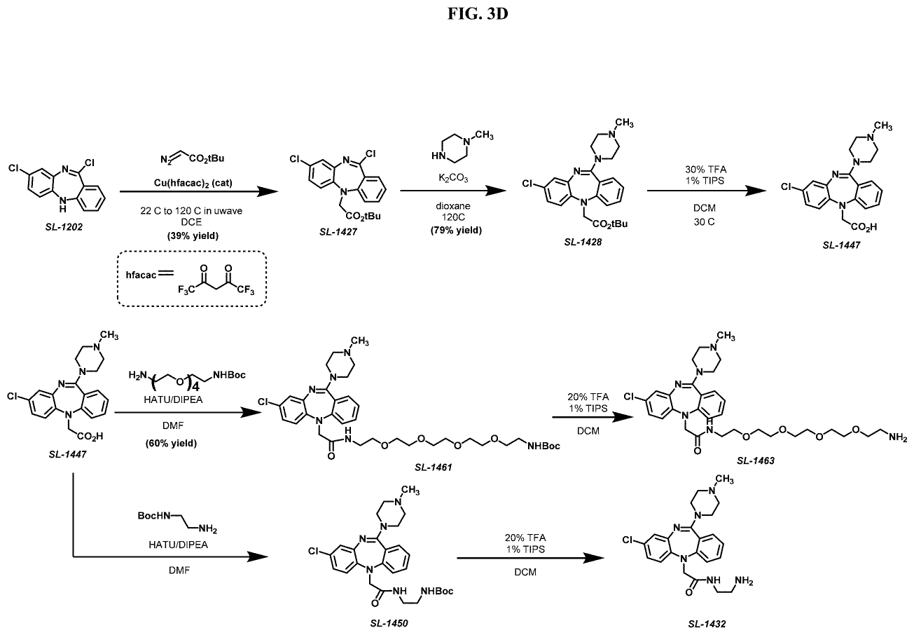

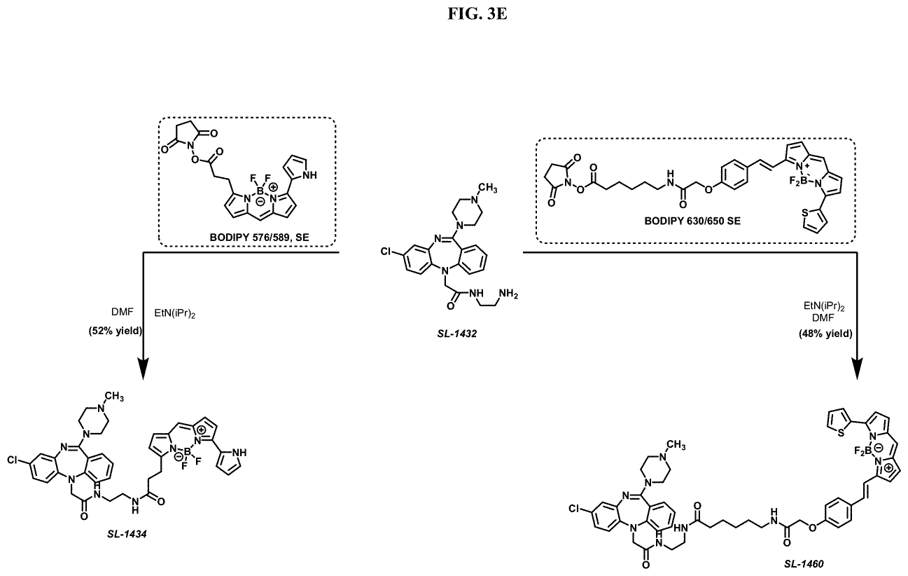

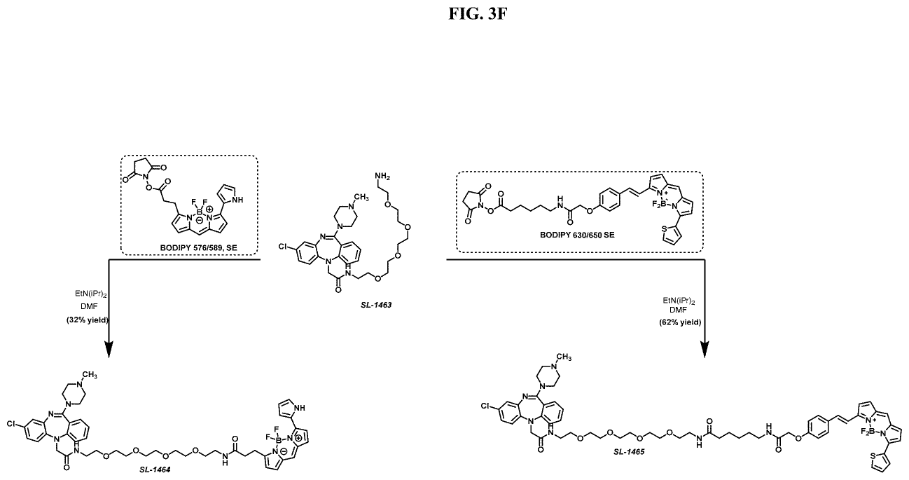

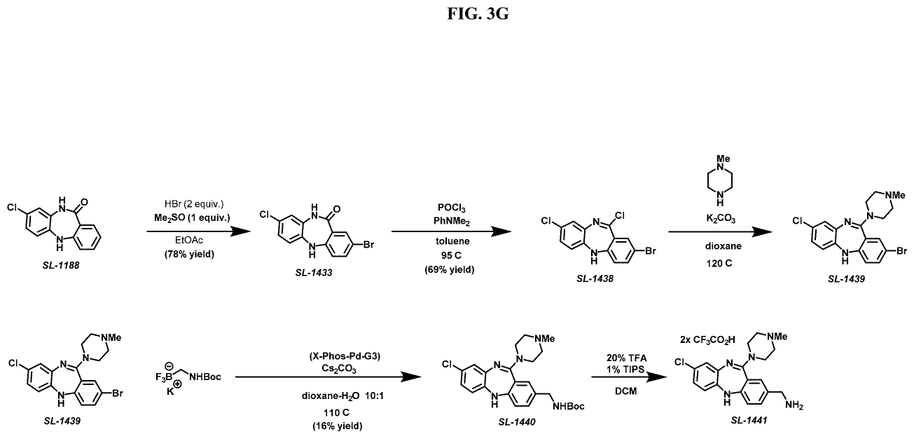

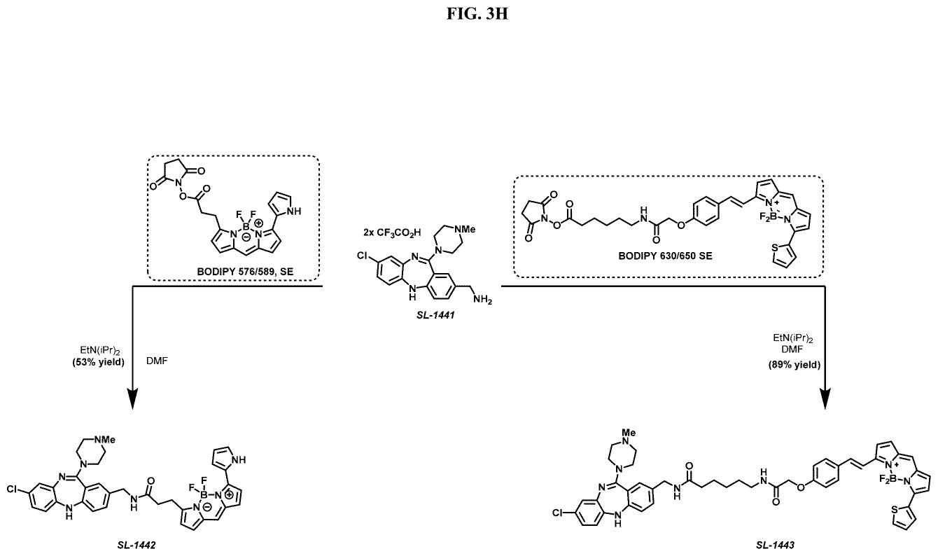

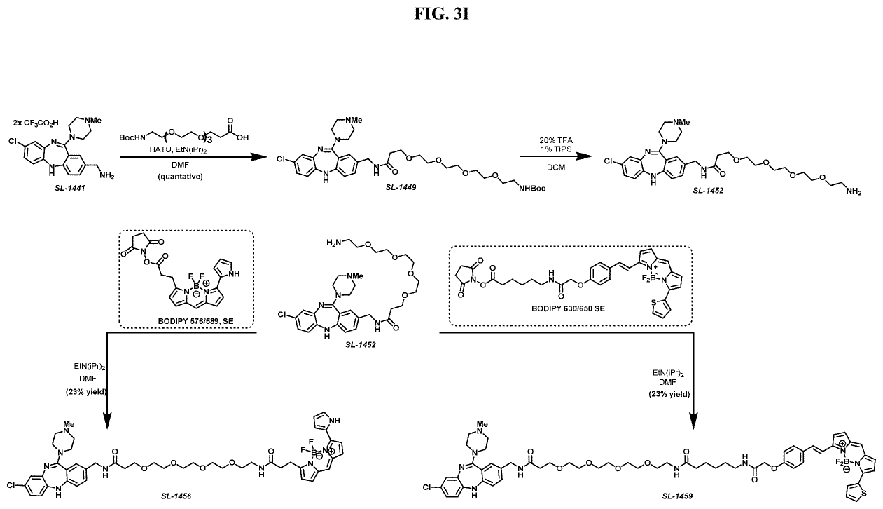

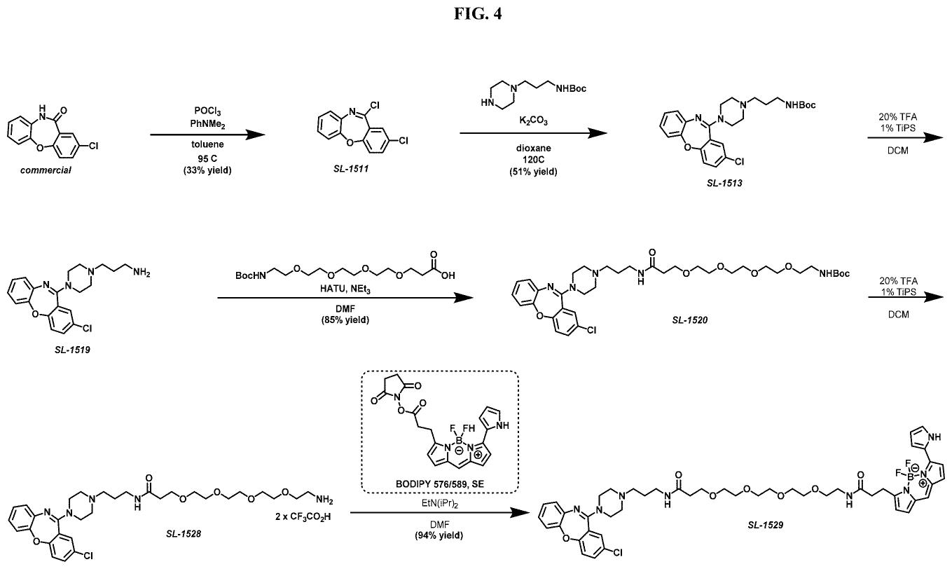

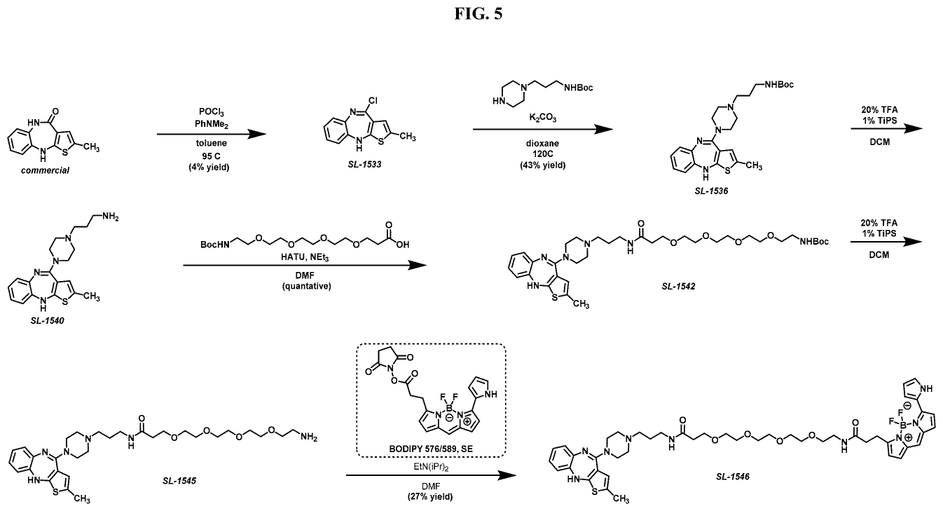

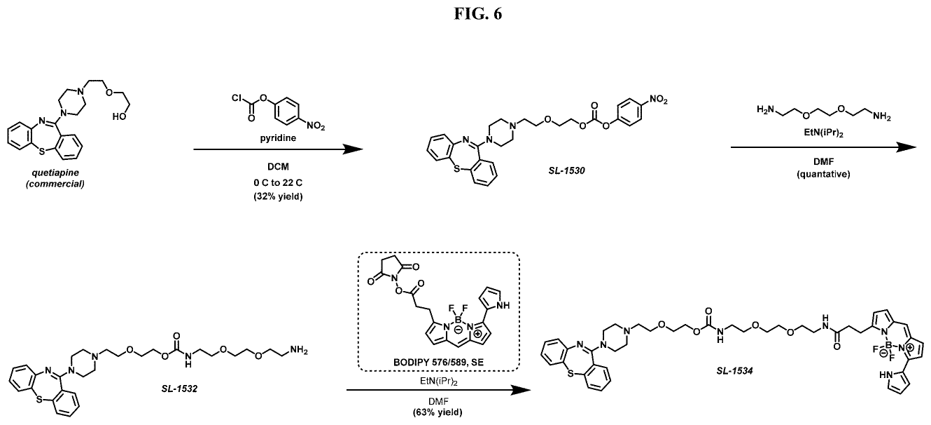

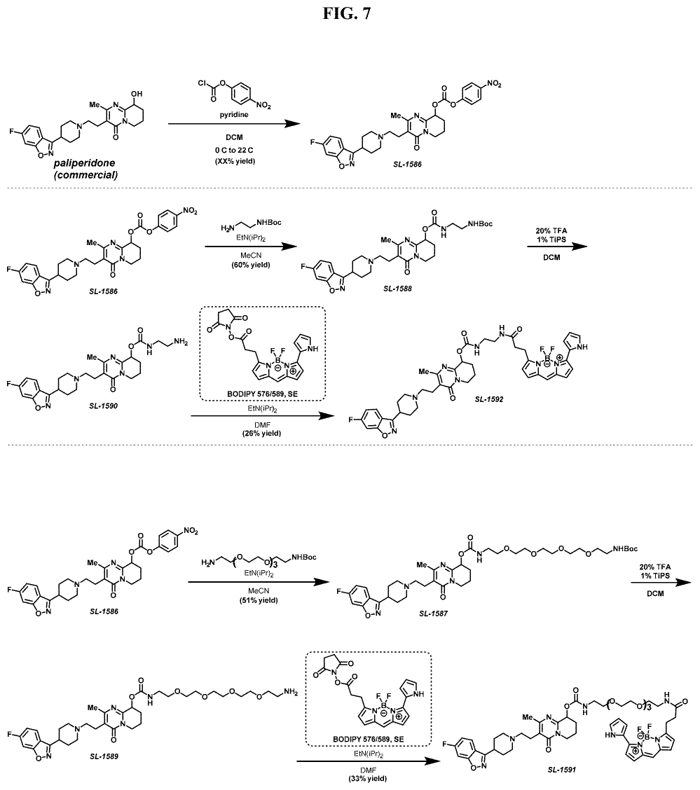

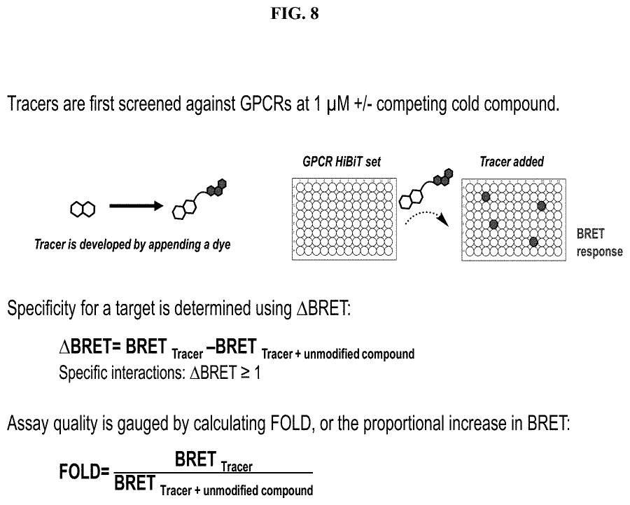

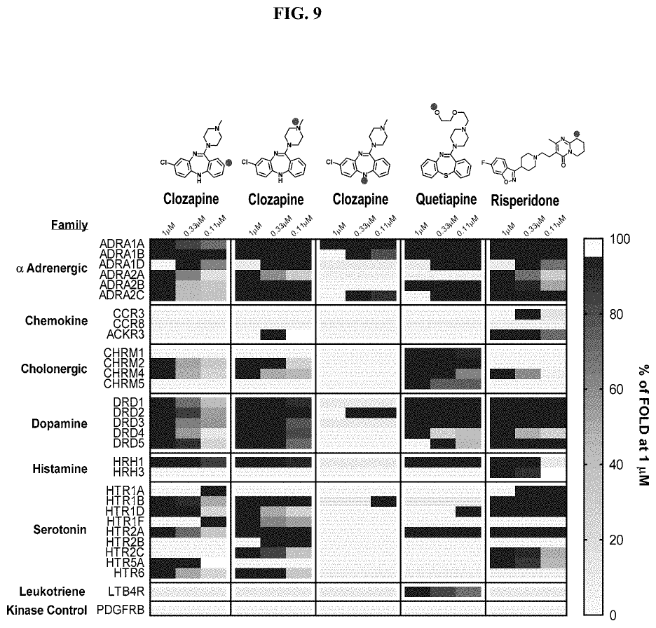

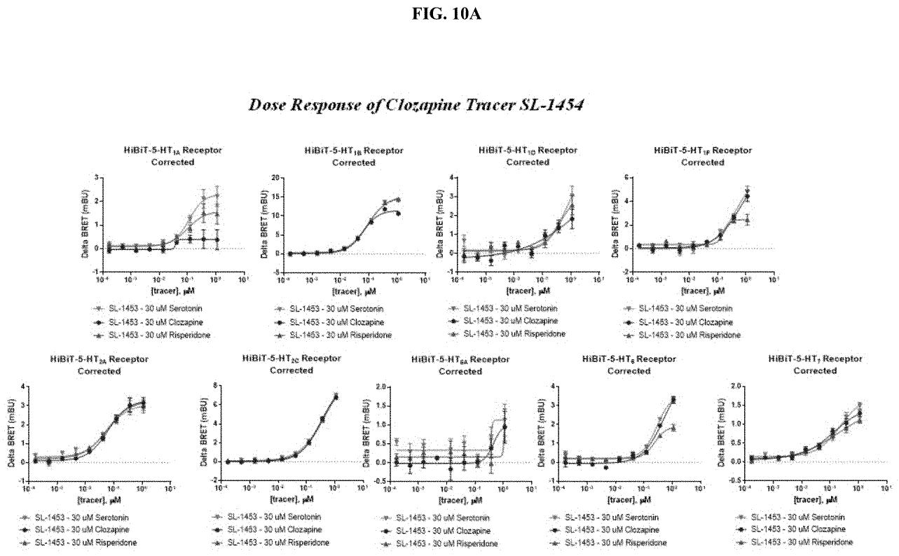

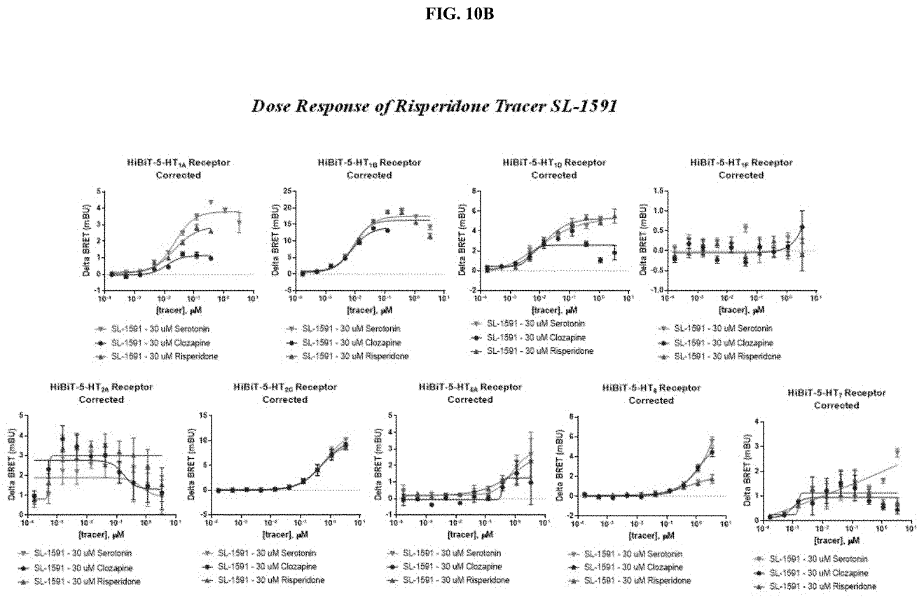

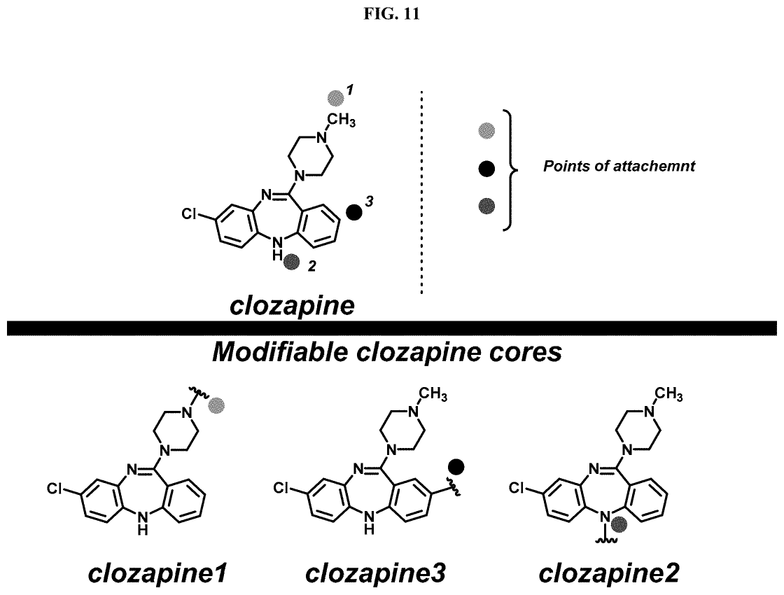

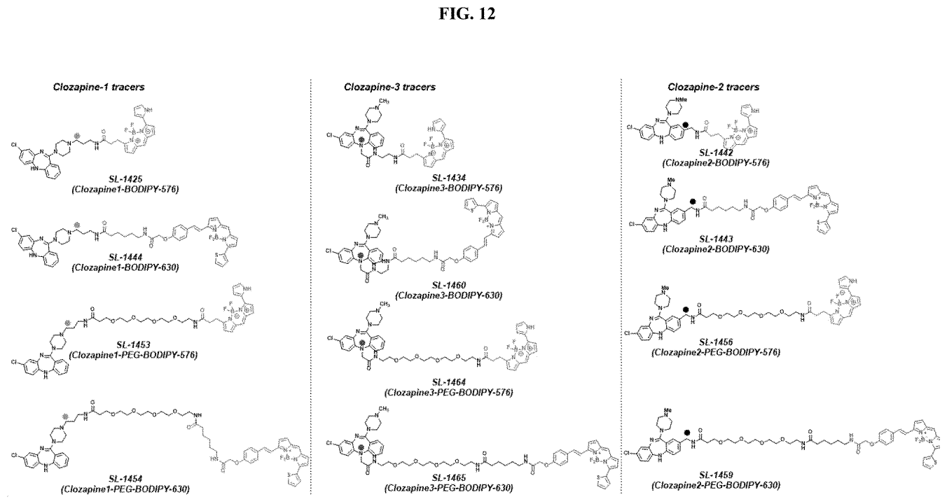

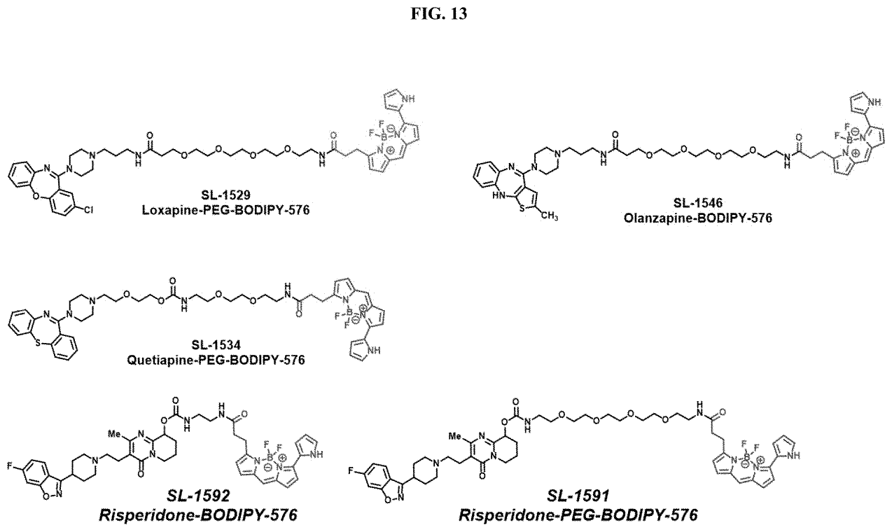

FIG. 1 demonstrates an exemplary BRET embodiment of the compositions and methods herein. A fluorescent signal is generated through energy transfer between HiBiT-tagged GPCR protein (through formation of bioluminescent HiBiT-LgBiT complex) and the fluorescent ligand, which allows real-time monitoring of GPCR-fluorescent ligand interactions. Displacement of the fluorescent ligand with a non-labeled ligand results in a loss of the BRET signal and allows both kinetic and binding data determination for the non-labeled compound. An advantage of this approach is the detection of only the interaction between fluorescent ligand and HiBiT-GPCR fusion. Any other interactions of the fluorescent ligand are not detected, thus significantly decreasing background and increasing sensitivity of BRET-signal detection (FIG. 2).

In some embodiments, provided in certain embodiments herein are systems comprising mutant proteins (e.g., mutant hydrolases (e.g., mutant dehalogenases)) that covalently bind their substrates (e.g., haloalkane substrates), for example, in U.S. Pat. Nos. 7,238,842; 7,425,436; 7,429,472; 7,867,726, each of which is herein incorporated by reference in their entireties. Such proteins may be provided as fusions with GPCRs. In other embodiments, such proteins are used to capture GPCRs bound to the agents described herein (e.g., wherein a functional group is a substrate for the mutant protein).

### EXPERIMENTAL

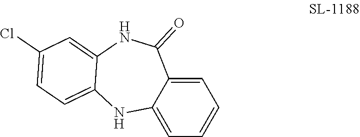 [SMILES: O=C1Nc2cc(Cl)ccc2Nc2ccccc21]

A 1 L flask, equipped with stir bar, was charged with 2-((2-amino-4-chlorophenyl)amino)benzoic acid (5.36 g, 20.4 mmol), HATU (8.53 g, 22.4 mmol), DMF (300 mL), and DIPEA (17.7 mL, 102 mmol). The resulting black solution was stirred at 22° C. for 20 hours, at which point, HPLC indicated complete consumption of the staring material, and the solvent removed under reduced pressure. The residue was purified by silica gel chromatography (0→50% EtOAc/hexanes) to provide 4.35 g (87% yield) of lactone SL-1188 as a light brown solid. ¹H NMR (400 MHz, DMSO-d₆) δ 9.91 (s, 1H), 7.97 (s, 1H), 7.68 (dd, J=7.9, 1.7 Hz, 1H), 7.35 (ddd, J=8.5, 7.2, 1.7 Hz, 1H), 6.99 (s, 3H), 6.97 (dd, J=8.2, 1.1 Hz, 1H), 6.93-6.82 (m, 1H).

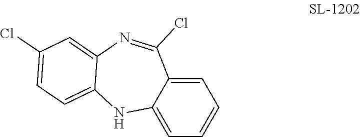 [SMILES: ClC1=Nc2cc(Cl)ccc2Nc2ccccc21]

Imidoyl chloride SL-1202 was prepared according to a published protocol: Ottesen, L. K.; Ek, F.; Olsson, R. Org. Lett. 2006, 8, 1771.

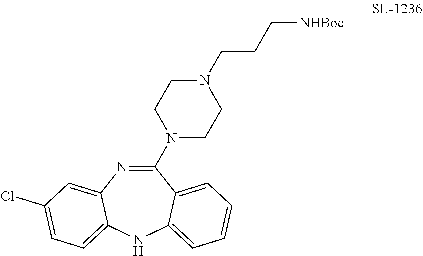 [SMILES: CC(C)(C)OC(=O)NCCCN1CCN(C2=Nc3cc(Cl)ccc3Nc3ccccc32)CC1]

A 10 mL microwave vial, equipped with stir bar, was charged with SL-1202 (56 mg, 0.21 mmol), tert-butyl (3-(piperazin-1-yl)propyl)carbamate (104 mg, 126 μmol), K₂CO₃ (74 mg, 0.53 mmol), and dioxane (4 mL). The vial was placed into a microwave reactor and heated to 120° C. for 1 hour. HPLC analysis confirmed consumption of the starting material, and then the solution was filtered and concentrated under reduced pressure. The crude residue was purified by flash chromatography (gradient elution, 0→20% MeOH/DCM) yielding 72 mg (73% yield) of amidine SL-1236 as a yellow solid. ¹H NMR (400 MHz, DMSO-d₆) δ 7.33 (td, J=7.8, 1.5 Hz, 1H), 7.26-7.10 (m, 2H), 7.09-6.92 (m, 2H), 6.87-6.71 (m, 3H), 2.94 (app. q, J=6.6 Hz, 2H), 2.42 (s, 3H partial overlap with DMSO-d₅), 2.30 (t, J=7.3 Hz, 2H partial overlap with DMSO-d₅-¹²C), 1.66-1.46 (m, 2H), 1.37 (s, 9H); HRMS (ESI) calc'd for C₂₅H₃₃ClN₅O₂ [M+H]⁺ 470.2323 found 470.2300.

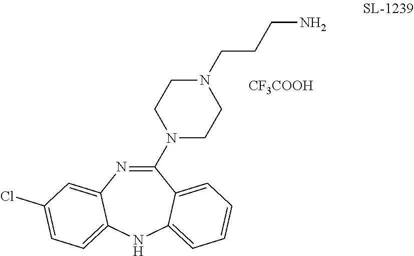 [SMILES: NCCCN1CCN(C2=Nc3cc(Cl)ccc3Nc3ccccc32)CC1 ; "CF3COOH"]

A 50 mL flask, equipped with stir bar, was charged with amidine SL-1236 (72 mg, 0.15 mmol) and a cleavage cocktail (10 mL, 85:15:1 DCM/TFA/TIPS). The resulting light yellow solution was stirred at 22° C. for 2 hours, at which point, HPLC indicated complete consumption of the staring material, and the solvent removed under reduced pressure. The residue was dissolved in 10 mL MeOH, and solvent removed under reduced pressure to provide 72 mg (97% yield) of primary amine SL-1239 as yellow oil. 1H NMR (400 MHz, DMSO-d₆) δ 7.81 (s, 2H), 7.50-7.36 (m, 2H), 7.32 (dd, J=7.8, 1.6 Hz, 1H), 7.14-6.99 (m, 2H), 6.99-6.76 (m, 3H), 3.96 (br. s, 1H), 3.54 (br. s. 1H), 3.31 (br. s, 5H), 2.53-2.51 (m, 2H, overlap with DMSO-d₅) 2.88 (m, 2H), 2.00-1.87 (m, 2H); HRMS (ESI) calc'd for C₂₀H₂₅ClN₅ [M+H]⁺ 370.1798 found 370.1790.

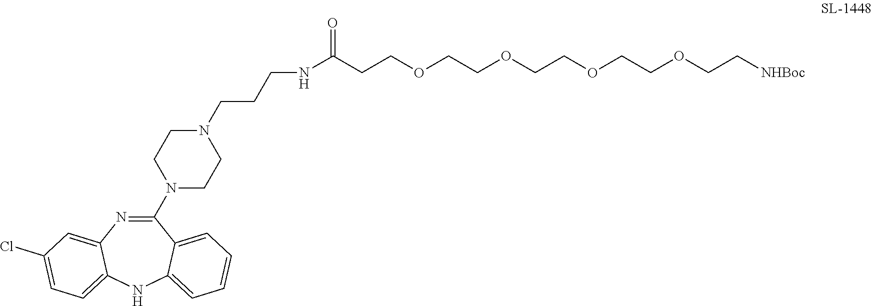 [SMILES: CC(C)(C)OC(=O)NCCOCCOCCOCCOCCC(=O)NCCCN1CCN(C2=Nc3cc(Cl)ccc3Nc3ccccc32)CC1]

A 25 mL flask, equipped with stir bar, was charged with SL-1239 (12 mg, 25 μmol), 2,2-dimethyl-4 oxo-3,8,11,14,17-pentaoxa-5-azaicosan-20-oic acid (14 mg, 37 μmol), HATU (12 mg, 31 μmol), NEt₃ (24 μL, 0.17 mmol), and DMF (6 mL). The resulting light yellow solution was stirred at 22° C. for 1 hour, at which point, HPLC indicated complete consumption of the staring material, and the solvent removed under reduced pressure. The crude residue was purified by preparative HPLC (C18, 5→95% MeCN/H₂O, 0.05% TFA) yielding 18 mg (quantative yield) of amide SL-1448 as a yellow oil. ¹H NMR (400 MHz, MeOD) δ 7.58-7.51 (m, 1H), 7.47 (dd, J=7.8, 1.6 Hz, 1H), 7-227.20 (m 1H), 7.20-7.08 (m, 3H), 6.97 (d, J=8.5 Hz, 1H), 3.94 (br. s, 4H), 3.76 (t, J=5.8 Hz, 2H), 3.61 (m, 12H), 3.49 (m, 6H), 3.38 (t, J=6.3 Hz, 2H), 3.28-3.03 (m, 4H), 2.50 (t, J=5.8 Hz, 2H), 2.11-1.86 (m, 2H), 1.43 (s, 9H). MS (ESI) calc'd for C₃₆H₅₄ClN₆O₇ [M+H]⁺ 717.37 found 717.58.

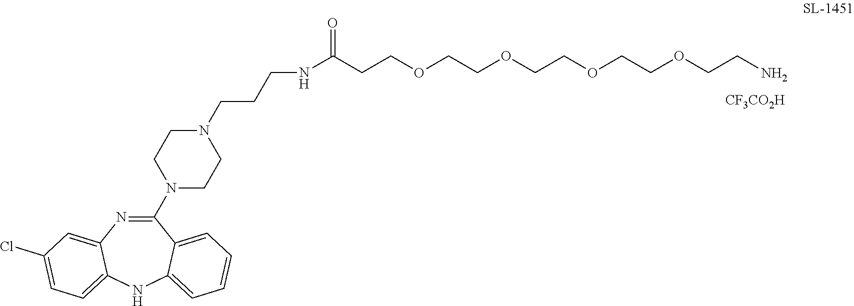 [SMILES: NCCOCCOCCOCCOCCC(=O)NCCCN1CCN(C2=Nc3cc(Cl)ccc3Nc3ccccc32)CC1 ; "CF3CO2H"]

A 25 mL flask, equipped with stir bar, was charged with SL-1248 (18 mg, 25 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and solvent removed under reduced pressure to provide 18 mg (quantative yield) of primary amine SL-1451 as a yellow oil. This material was further used without additional purification. HRMS (ESI) calc'd for C₃₁H₄₆ClN₅O₅ [M+H]⁺ 617.32 found 617.38.

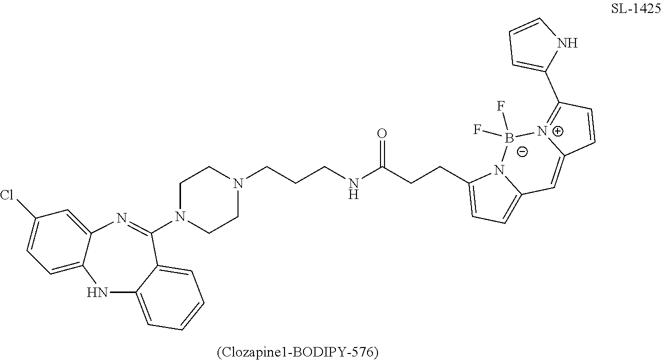 [SMILES: O=C(CCc1ccc2n1[B-](F)(F)[N+]1=C(c3ccc[nH]3)C=CC1=C2)NCCCN1CCN(C2=Nc3cc(Cl)ccc3Nc3ccccc32)CC1]

A 25 mL flask, equipped with stir bar, was charged with SL-1239 (5.7 mg, 12 μmol), BODIPY 576/589 SE (5.0 mg, 12 μmol), DIPEA (11 μL, 59 μmol), and DMF (8 mL). The resulting deep purple solution was stirred at 22° C. for 1 hour, at which point, HPLC indicated complete consumption of the staring material, and the reaction mixture was purified by preparative HPLC (C18, 5→95% MeCN/H₂O, 0.05% TFA) yielding 3 mg (38% yield) of amide SL-1425 as a deep purple film. ¹H NMR (400 MHz, MeOD) δ 7.40 (td, J=7.8, 1.5 Hz, 1H), 7.25 (dd, J=8.2, 1.5 Hz, 1H), 7.23-7.20 (m, 2H), 7.19 (s, 1H), 7.16 (d, J=4.6 Hz, 1H), 7.05-6.98 (m, 4H), 6.98-6.90 (m, 2H), 6.85 (d, J=8.4 Hz, 1H), 6.39 (d, J=3.9 Hz, 1H), 6.36 (dd, J=3.9, 2.5 Hz, 1H), 3.45-3.01 (br. s. 12H, overlap with CD₂HOD), 3.00 (t, J=7.5 Hz, 2H), 2.73 (t, J=7.2 Hz, 2H), 1.91 (p, J=6.8 Hz, 2H).

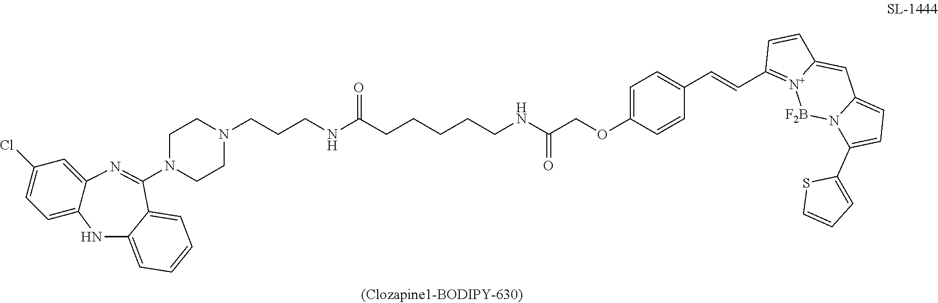

A 25 mL flask, equipped with stir bar, was charged with SL-1239 (5.7 mg, 12 μmol), BODIPY 630/650 SE (7.8 mg, 12 μmol), NEt₃ (8 μL, 60 μmol), and DMF (6 mL). The resulting deep purple solution was stirred at 22° C. for 1.5 hours, at which point, HPLC indicated complete consumption of the staring material, and the reaction mixture purified by preparative HPLC (C18, 5→95% MeCN/H₂O, 0.05% TFA) yielding 6.5 mg (60% yield) of amide SL-1444 as a deep blue-green film. ¹H NMR (400 MHz, MeOD) δ 8.19 (t, J=5.9 Hz, 1H, partially exchanged NH amide peak), 8.12 (dd, J=3.8, 1.1 Hz, 1H), 7.72-7.58 (m, 3H), 7.55 (d, J=4.4 Hz, 2H), 7.53-7.40 (m, 1H), 7.39-7.33 (m, 2H), 7.25-7.16 (m, 2H), 7.16-6.95 (m, 8H), 6.90 (d, J=8.5 Hz, 1H), 6.85 (d, J=4.3 Hz, 1H), 4.04-3.52 (br.s, 4H), 3.45-3.32 (br.s, 4H), 3.29-3.26 (m, 2H), 3.24 (t, J=6.5 Hz, 2H), 3.10 (t, J=7.6 Hz, 2H), 2.19 (t, J=7.4 Hz, 2H), 1.90 (p, J=6.6 Hz, 2H), 1.65-1.49 (m, 4H), 1.32-1.25 (m, 2H)); HRMS (ESI) calc'd for C₄₉H₅₁BClF₂N₈O₅S [M+H]⁺ 915.3554 found 915.3544.

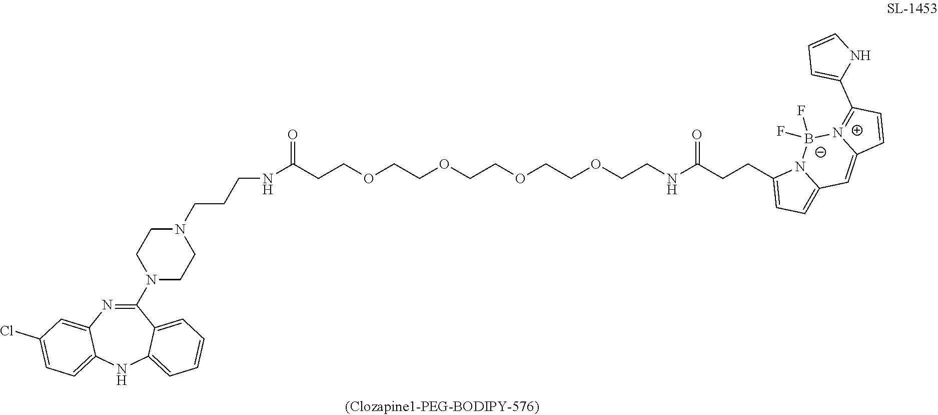 [SMILES: O=C(CCOCCOCCOCCOCCNC(=O)CCc1ccc2n1[B-](F)(F)[N+]1=C(c3ccc[nH]3)C=CC1=C2)NCCCN1CCN(C2=Nc3cc(Cl)ccc3Nc3ccccc32)CC1]

A 25 mL flask, equipped with stir bar, was charged with SL-1451 (7.0 mg, 11 μmol), BODIPY 576/589 SE (4.9 mg, 11 μmol), DIPEA (14 μL, 79 μmol), and DMF (6 mL). The resulting deep purple solution was stirred at 22° C. for 1 hour, at which point, HPLC indicated complete consumption of the staring material, and the reaction mixture purified by preparative HPLC (C18, 5→95% MeCN/H₂O, 0.05% TFA) yielding 1.8 mg (17% yield) of amide SL-1453 as a deep purple film. ¹H NMR (400 MHz, MeOD) δ 7.43 (ddd, J=8.0, 7.4, 1.6 Hz, 1H), 7.33 (dd, J=7.8, 1.5 Hz, 1H), 7.24 (s, 1H), 7.20 (m, 3H), 7.09 (dd, J=7.5, 1.2 Hz, 1H), 7.07-7.03 (m, 2H), 7.02 (d, J=4.6 Hz, 1H), 6.99 (dd, J=8.5, 2.4 Hz, 1H), 6.93 (d, J=4.0 Hz, 1H), 6.87 (d, J=8.4 Hz, 1H), 6.35 (m, 2H), 4.20-3.72 (br.s, 2H), 3.71 (t, J=5.8 Hz, 2H), 3.64-3.55 (m, 14H), 3.53 (m, 3H), 3.37 (t, J=5.5 Hz, 4H), 3.28 (m, 4H), 3.14 (d, J=14.3 Hz, 3H), 2.65 (dd, J=8.3, 7.1 Hz, 2H), 2.45 (t, J=5.8 Hz, 2H), 1.92 (p, J=6.8 Hz, 2H). HRMS (ESI) calc'd for C₄₇H₅₇BClF₂N₉O₆Na [M+Na]⁺ 950.4079 found 9050.4050.

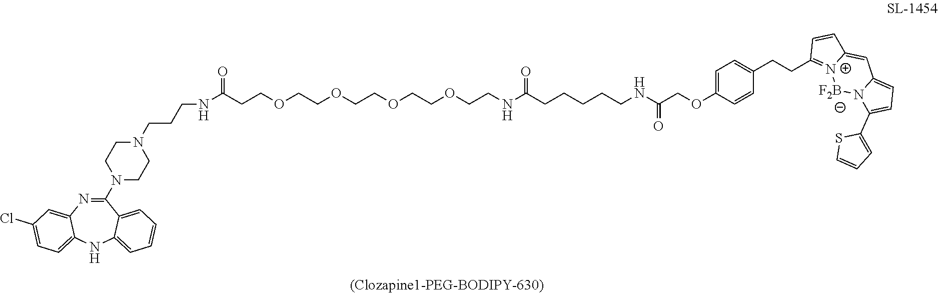

A 25 mL flask, equipped with stir bar, was charged with SL-1451 (7.0 mg, 11 μmol), BODIPY 630/650 SE (7.5 mg, 11 μmol), DIPEA (14 μL, 79 μmol), and DMF (6 mL). The resulting deep purple solution was stirred at 22° C. for 1.5 hours, at which point, HPLC indicated complete consumption of the staring material, and the reaction mixture purified by preparative HPLC (C18, 5→95% MeCN/H₂O, 0.05% TFA) yielding 5.7 mg (43% yield) of amide SL-1454 as a deep blue-green film. ¹H NMR (400 MHz, MeOD) δ 8.12 (dd, J=3.9, 1.1 Hz, 1H), 7.70-7.59 (m, 3H), 7.56 (d, J=4.0 Hz, 2H), 7.45 (ddd, J=8.1, 7.4, 1.6 Hz, 1H), 7.36 (m, 2H), 7.21 (m, 2H), 7.14 (br. d, J=4.5 Hz, 2H), 7.11 (dd, J=7.5, 1.2 Hz, 1H), 7.09-7.03 (m, 4H), 7.01 (dd, J=8.5, 2.4 Hz, 1H), 6.89 (d, J=8.5 Hz, 1H), 6.86 (d, J=4.3 Hz, 1H), 4.58 (s, 2H), 4.00-3.73 (br.s. 2H), 3.72 (t, J=5.8 Hz, 2H), 3.61-3.52 (m, 13H), 3.47 (d, J=5.6 Hz, 3H), 3.38 (br.s, 6H, overlap with CD₂HOD), 3.27 (m, 2H), 3.21-3.06 (m, 2H), 2.46 (t, J=5.8 Hz, 2H), 2.16 (t, J=7.4 Hz, 2H), 1.93 (p, J=6.7 Hz, 2H), 1.65-1.48 (m, 4H), 1.33-1.25 (m, 2H). HRMS (ESI) calc'd for C₆₀H₇₁BClF₂N₉O₈SNa [M+Na]⁺ 1184.4794 found 1184.4794.

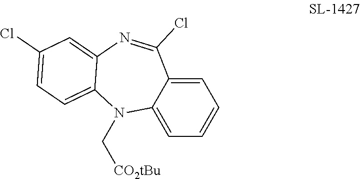 [SMILES: CC(C)(C)OC(=O)CN1c2ccc(Cl)cc2N=C(Cl)c2ccccc21]

A 10 mL microwave vial, equipped with stir bar, was charged with SL-1202 (18 mg, 68 μmol), Cu(hfacac)₂ (3.3 mg, 6.9 μmol), and DCE (2 mL). The vial was sealed, and tert-Butyl diazoacetate (28 μL, 0.21 mmol) slowly added to a stirred solution (gas evolution may occur). The vial was placed into a microwave reactor and heated to 120° C. for 1 minute (ramp to 120° C. takes 2 minutes). The cooled solution was purified by flash chromatography (gradient elution, 0→20% EtOAc/heptane, yielding 10 mg (39% yield) of ester SL-1427 as a yellow solid. ¹H NMR (400 MHz, CDCl₃) δ 7.65 (dd, J=7.8, 1.6 Hz, 1H), 7.42 (ddd, J=8.2, 7.4, 1.6 Hz, 1H), 7.22 (d, J=2.5 Hz, 1H), 7.19-7.08 (m, 2H), 6.91 (dd, J=8.2, 1.0 Hz, 1H), 6.82 (d, J=8.6 Hz, 1H), 4.39 (d, J=16.4 Hz, 1H), 4.26 (d, J=16.3 Hz, 1H), 1.30 (s, 9H); HRMS (ESI) calc'd for C₁₉H₁₉Cl₂N₂O₂ [M+H]⁺ 377.0818 found 377.0809.

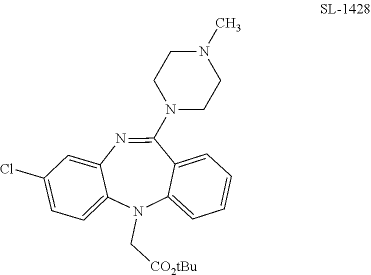 [SMILES: CN1CCN(C2=Nc3cc(Cl)ccc3N(CC(=O)OC(C)(C)C)c3ccccc32)CC1]

A 20 mL microwave vial, equipped with stir bar, was charged with SL-1427 (54 mg, 0.14 mmol), 1-methylpiperazine (80 μL, 0.72 mmol), K₂CO₃ (50 mg, 0.36 mmol), and dioxane (10 mL). The vial was placed into a microwave reactor and heated to 120° C. for 2 hours. The cooled solution was filtered, and the solvent removed under reduced pressure. The crude residue was purified by flash chromatography (gradient elution, 0→30% MeOH/DCM, yielding 50 mg (79% yield) of amidine SL-1428 as a grey solid. ¹H NMR (400 MHz, MeOD) δ 7.42 (ddd, J=8.2, 7.3, 1.6 Hz, 1H), 7.36-7.23 (m, 1H), 7.23-7.04 (m, 2H), 6.98 (dd, J=1.9, 1.0 Hz, 1H), 6.94-6.80 (m, 2H), 4.53 (d, J=16.8 Hz, 1H), 4.25 (d, J=16.7 Hz, 1H), 3.61-3.40 (m, 4H), 2.69-2.44 (m, 4H), 2.34 (s, 3H), 1.38 (s, 9H); HRMS (ESI) calc'd for C₂₄H₃₀ClN₄O₂ [M+H]⁺ 441.2057 found 441.2042.

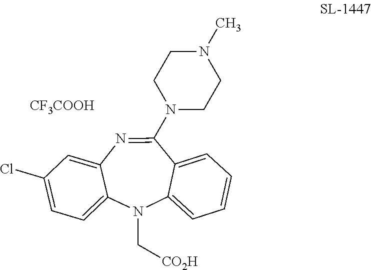 [SMILES: "CF3COOH" ; CN1CCN(C2=Nc3cc(Cl)ccc3N(CC(=O)O)c3ccccc32)CC1]

A 250 mL flask, equipped with stir bar, was charged with amidine SL-1428 (5.4 mg, 12 μmol) and a cleavage cocktail (10 mL, 75:25:1 DCM/TFA/TIPS). The resulting light yellow solution was stirred at 22° C. for 3 hours, at which point, HPLC indicated complete consumption of the staring material, and the solvent removed under reduced pressure. The residue was dissolved in 10 mL MeOH, and the solvent removed under reduced pressure to provide carboxylic acid SL-1447 as yellow oil, which was used in the following steps without further purification. ¹H NMR (400 MHz, DMSO-d₆) 7.62 (ddd, J=8.7, 7.3, 1.6 Hz, 1H), 7.50 (dd, J=7.8, 1.6 Hz, 1H), 7.38 (dd, J=8.3, 1.0 Hz, 1H), 7.29 (td, J=7.6, 1.1 Hz, 1H), 7.26-7.19 (m, 2H), 7.16 (d, J=8.7 Hz, 1H), 4.79 (d, J=17.5 Hz, 1H), 4.48 (d, J=17.6 Hz, 1H), 4.29-3.71 (m, 4H), 3.68-3.38 (m, 4H), 2.98 (s, 3H).

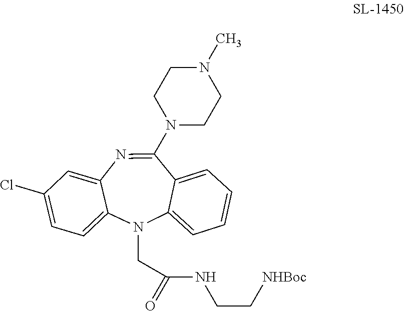 [SMILES: CN1CCN(C2=Nc3cc(Cl)ccc3N(CC(=O)NCCNC(=O)OC(C)(C)C)c3ccccc32)CC1]

A 25 mL flask, equipped with stir bar, was charged with SL-1447 (20 mg, 52 μmol), tert-butyl (2-aminoethyl)carbamate (10 mg, 65 μmol), HATU (25 mg, 65 μmol), DIPEA (65 μL, 0.36 mmol), and DMF (6 mL). The resulting light yellow solution was stirred at 22° C. for 3 hours, at which point, HPLC indicated complete consumption of the staring material, and the solvent removed under reduced pressure. The crude residue was purified by flash chromatography (gradient elution, 0→20% MeOH/DCM, yielding 3 mg (11% yield) of amide SL-1450 as yellow oil. ¹H NMR (400 MHz, MeOD) δ 7.47 (ddd, J=8.6, 7.3, 1.6 Hz, 1H), 7.35 (dd, J=7.7, 1.6 Hz, 1H), 7.28-7.12 (m, 2H), 7.03 (d, J=2.2 Hz, 1H), 7.01-6.88 (m, 2H), 4.40 (d, J=15.8 Hz, 1H), 4.31 (d, J=15.7 Hz, 1H), 3.82-3.38 (m, 4H), 3.19 (m, 2H), 3.08-2.95 (m, 2H), 2.68 (s, 2H), 2.59 (m, 2H), 2.40 (s, 3H), 1.39 (s, 9H); HRMS (ESI) calc'd for C₂₇H₃₆ClN₆O₃ [M+H]⁺ 527.2537 found 527.2528.

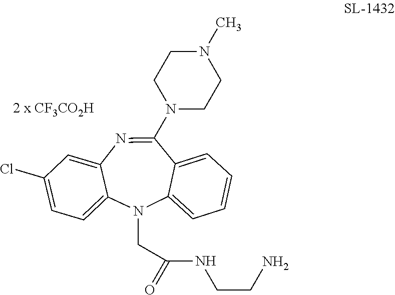 [SMILES: CN1CCN(C2=Nc3cc(Cl)ccc3N(CC(=O)NCCN)c3ccccc32)CC1]

A 25 mL flask, equipped with stir bar, was charged with amide SL-1450 (6 mg, 11 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide primary amine SL-1432 as yellow oil, which was used in the following steps without further purification. ¹H NMR (400 MHz, MeOD) δ 7.98 (s, 3H), 7.63 (ddd, J=8.6, 7.4, 1.6 Hz, 1H), 7.52 (dd, J=7.8, 1.6 Hz, 1H), 7.39-7.29 (m, 2H), 7.27 (d, J=2.4 Hz, 1H), 7.22 (dd, J=8.7, 2.4 Hz, 1H), 7.14 (d, J=8.7 Hz, 1H), 4.60 (d, J=15.7 Hz, 1H), 4.45 (d, J=15.8 Hz, 1H), 3.97 (br. s, 4H), 3.70-3.47 (m, 4H), 3.44 (td, J=6.2, 4.6 Hz, 2H), 3.05-3.01 (m, 2H), 3.00 (s, 4H); MS (ESI) calc'd for C₂₂H₂₈ClN₆O [M+H]⁺ 427.20 found 427.09.

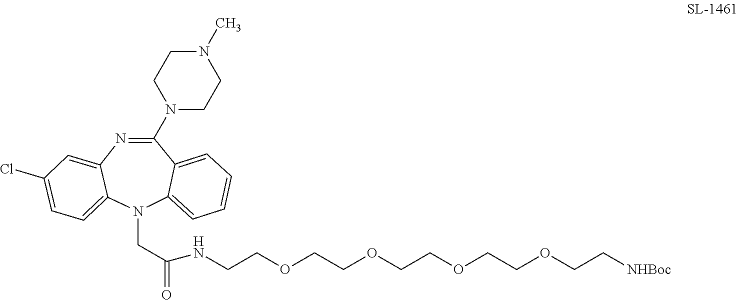 [SMILES: CN1CCN(C2=Nc3cc(Cl)ccc3N(CC(=O)NCCOCCOCCOCCOCCNC(=O)OC(C)(C)C)c3ccccc32)CC1]

A 25 mL flask, equipped with stir bar, was charged with SL-1447 (43 mg, 0.11 mmol), tert-butyl (14-amino-3,6,9,12-tetraoxatetradecyl)carbamate (56 mg, 0.17 mmol), HATU (53 mg, 0.14 mmol), DIPEA (140 μL, 0.78 mmol), and DMF (8 mL). The resulting light yellow solution was stirred at 22° C. for 21 hours, at which point, HPLC indicated complete consumption of the staring material, and the solvent removed under reduced pressure. The crude residue was purified by preparative HPLC (C18, 5→95% MeCN/H₂O, 0.05% TFA), yielding 47 mg (60% yield) of carbamate SL-1461 as a clear oil. ¹H NMR (400 MHz, MeOD) δ 7.54 (ddd, J=8.2, 7.3, 1.6 Hz, 1H), 7.42 (dd, J=7.7, 1.6 Hz, 1H), 7.30-7.21 (m, 2H), 7.15-7.06 (m, 2H), 7.04 (d, J=8.6 Hz, 1H), 4.54 (d, J=15.5 Hz, 1H), 4.31 (d, J=15.6 Hz, 1H), 3.73-3.56 (m, 11H), 3.56-3.39 (m, 10H), 3.21 (t, J=5.7 Hz, 2H), 2.99 (s, 3H), 1.43 (s, 9H); MS (ESI) calc'd for C₃₅H₅₂ClN₆O₇ [M+H]⁺ 703.36 found 703.44.

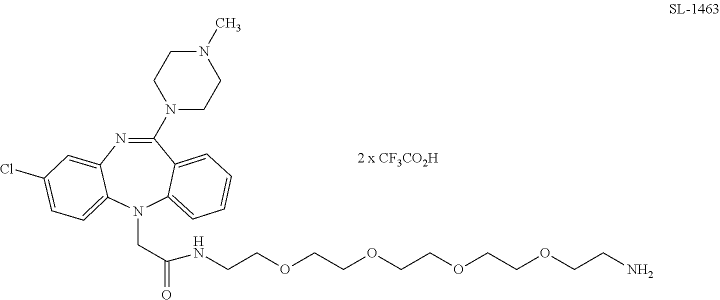 [SMILES: CN1CCN(C2=Nc3cc(Cl)ccc3N(CC(=O)NCCOCCOCCOCCOCCN)c3ccccc32)CC1]

A 25 mL flask, equipped with stir bar, was charged with SL-1461 (47 mg, 67 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 56 mg (quantative yield) of primary amine SL-1451 as a yellow oil. This material was further used without additional purification. HRMS (ESI) calc'd for C₃₀H₄₄ClN_{6NH2}O₅ [M+H]⁺ 603.31 found 603.24.

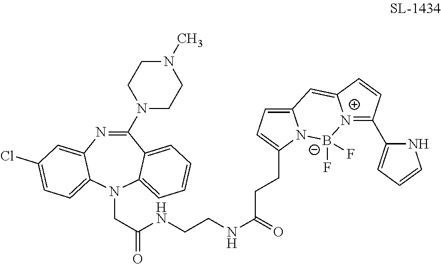 [SMILES: CN1CCN(C2=Nc3cc(Cl)ccc3N(CC(=O)NCCNC(=O)CCc3ccc4n3[B-](F)(F)[N+]3=C(c5ccc[nH]5)C=CC3=C4)c3ccccc32)CC1]

A 25 mL flask, equipped with stir bar, was charged with SL-1432 (4.8 mg, 11 μmol), BODIPY 576/589 SE (4.8 mg, 11 μmol), DIPEA (14 μL, 78 μmol), and DMF (10 mL). The resulting deep purple solution was stirred at 22° C. for 3 hours, at which point, HPLC indicated complete consumption of the staring material, and the reaction mixture purified by preparative HPLC (C18, 5→95% MeCN/H₂O, 0.05% TFA) yielding 4.3 mg (52% yield) of amide SL-1434 as a deep purple film. ¹H NMR (400 MHz, MeOD) δ 7.42 (ddd, J=8.3, 7.3, 1.6 Hz, 1H), 7.34 (dd, J=7.7, 1.6 Hz, 1H), 7.28-7.16 (m, 4H), 7.16-7.10 (m, 3H), 7.10-7.01 (m, 2H), 6.98 (d, J=8.7 Hz, 1H), 6.88 (d, J=4.0 Hz, 1H), 6.37 (dd, J=3.9, 2.5 Hz, 1H), 6.30 (d, J=4.0 Hz, 1H), 4.23 (s, 2H), 4.09-3.55 (br. s, 4H), 3.55-3.40 (br.s, 4H), 3.40-3.32 (m, 2H), 3.27 (t, J=7.2 Hz, 1H), 3.20 (t, J=7.2 Hz, 1H), 3.17-3.08 (m, 2H), 2.93 (s, 3H), 2.59 (t, J=7.4 Hz, 2H); HRMS (ESI) calc'd for C₃₈H₄₀BClF₂N₉O₂ [M+H]+ 738.3055 found 738.3055.

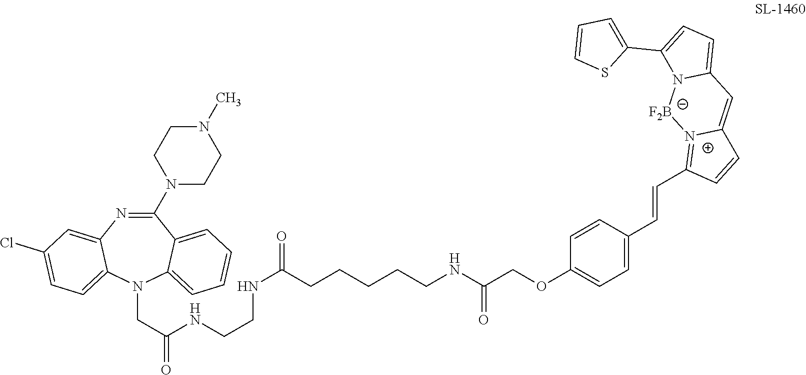

A 25 mL flask, equipped with stir bar, was charged with SL-1432 3.0 mg, 7 μmol), BODIPY 630/650 SE (4.6 mg, 7 μmol), DIPEA (9 μL, 50 μmol), and DMF (8 mL). The resulting deep purple solution was stirred at 22° C. for 1 hour, at which point, HPLC indicated complete consumption of the staring material, and the reaction mixture purified by preparative HPLC (C18, 5→95% MeCN/H₂O, 0.05% TFA) yielding 3.3 mg (48% yield) of amide SL-1460 as a deep blue-green film. ¹H NMR (400 MHz, MeOD) δ 8.12 (dd, J=3.8, 1.1 Hz, 1H), 7.66-7.58 (m, 3H), 7.55 (m, 2H), 7.53-7.48 (m, 1H), 7.42-7.38 (m, 1H), 7.38 (s, 1H), 7.21 (ddd, J=8.9, 6.4, 4.4 Hz, 4H), 7.14 (t, J=4.3 Hz, 2H), 7.11 (d, J=2.4 Hz, 1H), 7.08-7.02 (m, 3H), 6.99 (d, J=8.7 Hz, 1H), 6.86 (d, J=4.3 Hz, 1H), 4.58 (s, 2H), 4.44 (d, J=15.6 Hz, 1H), 4.25 (d, J=15.6 Hz, 1H), 4.10-3.54 (m, 4H), 3.38 (d, J=26.4 Hz, 5H), 3.26 (td, J=6.9, 2.1 Hz, 3H), 3.21-3.14 (m, 3H), 2.94 (s, 3H), 2.09 (t, J=7.4 Hz, 2H), 1.63-1.49 (m, 4H), 1.39-1.06 (m, 2H); HRMS (ESI) calc'd for C₅₁H₅₄BClF₂N₉O₄S [M+H]⁺ 972.3769 found 972.3769.

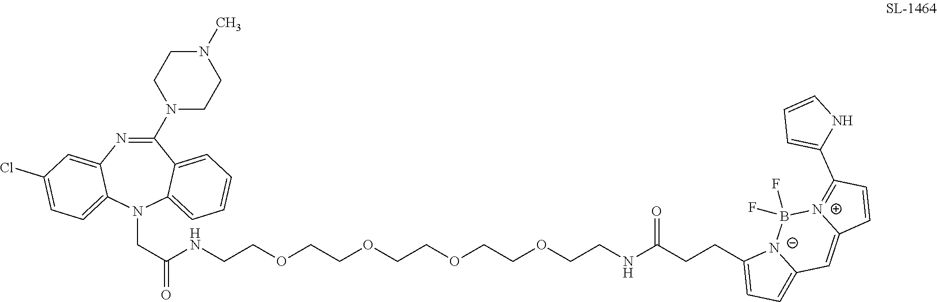

A 25 mL flask, equipped with stir bar, was charged with SL-1463 (10 mg, 17 μmol), BODIPY 576/589 SE (7.1 mg, 17 μmol), DIPEA (20 μL, 0.12 mmol), and DMF (6 mL). The resulting deep purple solution was stirred at 22° C. for 18 hours, at which point, HPLC indicated complete consumption of the staring material, and the reaction mixture purified by preparative HPLC (C18, 5→95% MeCN/H₂O, 0.05% TFA), yielding 4.8 mg (32% yield) of amide SL-1464 as a deep purple film. ¹H NMR (400 MHz, MeOD) δ 7.53 (ddd, J=8.2, 7.3, 1.6 Hz, 1H), 7.40 (dd, J=7.7, 1.6 Hz, 1H), 7.29-7.17 (m, 6H), 7.15-7.06 (m, 2H), 7.06-6.97 (m, 2H), 6.93 (d, J=4.0 Hz, 1H), 6.34 (td, J=4.3, 3.8, 1.8 Hz, 2H), 4.48 (d, J=15.6 Hz, 1H), 4.28 (d, J=15.6 Hz, 1H), 4.14-3.65 (m, 4H), 3.64-3.56 (m, 8H), 3.56-3.33 (m, 14H), 2.95 (s, 3H), 2.72-2.58 (m, 2H); HRMS (ESI) calc'd for C₄₆H₅₆BClF₂N₉O₆ [M+H]⁺ 914.4103 found 914.4111.

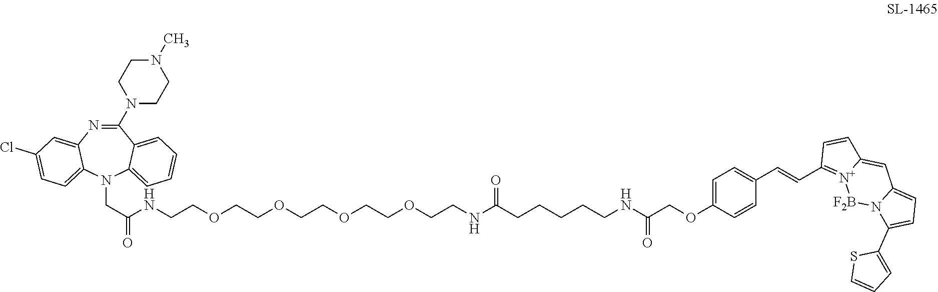

A 25 mL flask, equipped with stir bar, was charged with SL-1463 (10 mg, 17 μmol), BODIPY 630/650 SE (11 mg, 17 μmol), DIPEA (20 μL, 0.12 mmol), and DMF (6 mL). The resulting deep purple solution was stirred at 22° C. for 18 hours, at which point, HPLC indicated complete consumption of the staring material, and the reaction mixture purified by preparative HPLC (C18, 5→95% MeCN/H₂O, 0.05% TFA), yielding 12 mg (62% yield) of amide SL-1465 as a deep blue-green film. ¹H NMR (400 MHz, MeOD) δ 8.12 (dd, J=3.9, 1.1 Hz, 1H), 7.66-7.56 (m, 4H), 7.55 (dd, J=4.1, 2.7 Hz, 2H), 7.45 (dd, J=7.8, 1.6 Hz, 1H), 7.37 (s, 1H), 7.31-7.22 (m, 2H), 7.22-7.17 (m, 3H), 7.17-7.11 (m, 3H), 7.10-6.99 (m, 3H), 6.86 (d, J=4.3 Hz, 1H), 4.57 (s, 2H), 4.51 (d, J=15.5 Hz, 1H), 4.32 (d, J=15.5 Hz, 1H), 3.89 (d, J=41.1 Hz, 4H), 3.64-3.36 (m, 20H), 3.30-3.21 (m, 2H), 2.96 (s, 3H), 2.16 (t, J=7.4 Hz, 2H), 1.56 (dp, J=21.9, 7.3 Hz, 4H), 1.37-1.14 (m, 2H); HRMS (ESI) calc'd for C₅₉H₇₀BClF₂N₉O₈S [M+H]⁺ 1148.4818 found 1148.4887.

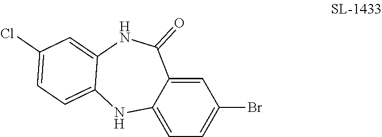 [SMILES: O=C1Nc2cc(Cl)ccc2Nc2ccc(Br)cc21]

A 25 mL flask, equipped with stir bar, was charged with SL-1188 (29 mg, 0.12 mmol), EtOAc (4 mL), and DMSO (10 mg, 0.13 mmol). Upon addition of HBr (33 wt % in H₂O, 58 mg, 0.24 mmol), precipitation occurs. The resulting suspension was heated at 50° C. for 1 hour, at which point, HPLC indicated complete consumption of the staring material, and the solvent removed under reduced pressure. The residue was purified by silica gel chromatography (0→60% EtOAc/hexanes) to provide 3 mg (78% yield) of arylbromide SL-1433 as a yellow solid. ¹H NMR (400 MHz, DMSO-d₆) δ 10.04 (s, 1H), 8.17 (s, 1H), 7.75 (d, J=2.6 Hz, 1H), 7.51 (dd, J=8.7, 2.6 Hz, 1H), 7.02 (dd, J=8.3, 2.3 Hz, 1H), 7.00 (d, J=2.2 Hz, 1H), 6.97 (d, J=8.4 Hz, 1H), 6.93 (d, J=8.6 Hz, 1H); ¹³C NMR (100 MHz, DMSO) δ 166.2, 149.0, 137.9, 136.0, 134.2, 130.8, 126.6, 124.2, 123.9, 121.3, 121.2, 120.6, 112.1; HRMS (ESI) calc'd for C₁₃H₉BrClN₂O [M+H]⁺ 322.9587 found 322.9585.

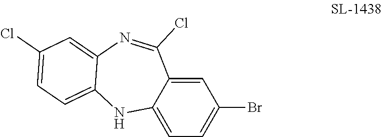 [SMILES: ClC1=Nc2cc(Cl)ccc2Nc2ccc(Br)cc21]

A 25 mL flask, equipped with stir bar, was charged with SL-1433 (145 mg, 593 μmol), N,N-dimethylaniline (0.30 mL, 2.4 mmol), POCl₃ (166 μL, 1.78 mmol), and toluene (5 mL). The resulting suspension was heated to 95° C. for 2.5 hours, and a dark brown solution formed. Solvent was removed under reduced pressure, and the residue dissolved in a mixture of dioxane (5 mL) and aqueous 2M Na₂CO₃ (7 mL). The resulting solution was heated at 80° C. for 45 minutes, dioxane removed under reduced pressure, and the residue extracted in EtOAc (3×25 mL). Combined EtOAc solutions were dried with MgSO₄, filtered, and the solvent removed under reduced pressure. The residue was purified by silica gel chromatography (0→30% EtOAc/hexanes) to provide 107 mg (69% yield) of imidoyl chloride SL-1438 as a yellow solid. ¹H NMR (400 MHz, DMSO-d₆) δ 7.80 (s, 1H), 7.64-7.49 (m, 2H), 7.19 (dd, J=8.5, 2.5 Hz, 1H), 7.07 (d, J=2.4 Hz, 1H), 6.87 (d, J=8.6 Hz, 1H), 6.84 (dt, J=8.7, 1.1 Hz, 1H; HRMS (ESI) calc'd for C₁₃H₈BrClN₂ [M+H]⁺ 340.9248 found 340.9244.

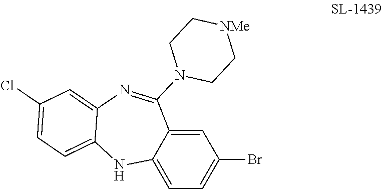 [SMILES: CN1CCN(C2=Nc3cc(Cl)ccc3Nc3ccc(Br)cc32)CC1]

A 20 mL microwave vial, equipped with stir bar, was charged with SL-1438 (105 mg, 307 μmol), 1-methylpiperazine (170 μL, 1.5 mmol), K₂CO₃ (127 mg, 921 μmol), and dioxane (10 mL). The vial was placed into a microwave reactor and heated to 120° C. for 2 hours. The cooled solution was filtered, and the solvent removed under reduced pressure. The crude residue was purified by flash chromatography (gradient elution, 0→30% MeOH/DCM, yielding 120 mg (96% yield) of amidine SL-1439 as a yellowish solid. ¹H NMR (400 MHz, MeOD) δ 7.53 (dd, J=8.5, 2.4 Hz, 1H), 7.36 (s, 1H), 7.30 (d, J=2.4 Hz, 1H), 6.99 (d, J=8.6 Hz, 1H), 6.90-6.85 (m, 3H), 2.39 (t, J=4.9 Hz, 4H), 2.21 (s, 3H); HRMS (ESI) calc'd for C₁₈H₁₉BrClN₄ [M+H]⁺ 405.0482 found 405.0472.

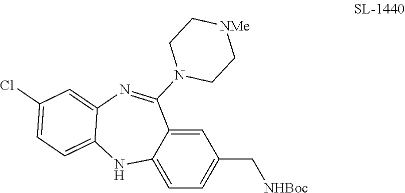 [SMILES: CN1CCN(C2=Nc3cc(Cl)ccc3Nc3ccc(CNC(=O)OC(C)(C)C)cc32)CC1]

A 35 mL microwave vial, equipped with stir bar, was charged under Ar with SL-1439 (78 mg, 0.19 mmol), potassium (Boc-amino-methyl)trifluoroborate, X-Phod-Pd-G3 (25 mg, 30 μmol), Cs₂CO₃, degassed dioxane (12 mL), and degassed water (1 mL). The vial was placed into a microwave reactor and heated to 110° C. for 16 hours. The cooled solution was filtered, and solvents removed under reduced pressure. The crude residue was purified by flash chromatography (gradient elution, 0→30% MeOH/DCM, yielding 140 mg (16% yield) of carbamate SL-1440 as a green oily solid. ¹H NMR (400 MHz, MeOD) δ 7.24 (dd, J=8.2, 2.1 Hz, 1H), 7.19 (d, J=2.1 Hz, 1H), 6.98-6.88 (m, 2H), 6.84 (dd, J=8.4, 2.4 Hz, 1H), 6.78 (d, J=8.4 Hz, 1H), 4.13 (s, 2H), 3.43 (s, 4H), 2.59 (s, 4H), 2.37 (s, 3H), 1.45 (s, 9H); HRMS (ESI) calc'd for C₂₄H₃₁ClN₅O₂ [M+H]⁺ 456.2166 found 456.2156.

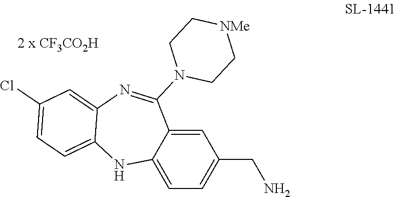 [SMILES: CN1CCN(C2=Nc3cc(Cl)ccc3Nc3ccc(CN)cc32)CC1]

A 25 mL flask, equipped with stir bar, was charged with carbamate SL-1441 (14 mg, 31 μmol) and a cleavage cocktail (7 mL, 80:20:1 DCM/TFA/TIPS). The resulting light yellow solution was stirred at 22° C. for 1.5 hours, at which point, HPLC indicated complete consumption of the staring material, and the solvent removed under reduced pressure. The residue was dissolved in 10 mL MeOH, and the solvent removed under reduced pressure to provide primary amine SL-1441 as yellow oil, which was further used without additional purification. MS (ESI) calc'd for C₁₉H₂₃ClN₅ [M+H]⁺ 356.16 found 356.06.

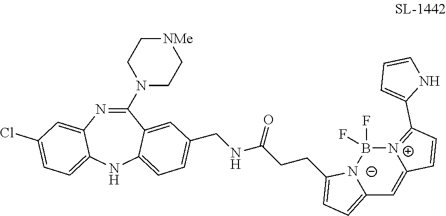

A 25 mL flask, equipped with stir bar, was charged with SL-1442 (3.5 mg, 7.5 μmol), BODIPY 576/589 SE (3.2 mg, 7.5 μmol), DIPEA (7 μL, 37 μmol), and DMF (6 mL). The resulting deep purple solution was stirred at 22° C. for 1 hour, at which point, HPLC indicated complete consumption of the staring material, and the reaction mixture purified by preparative HPLC (C18, 5→95% MeCN/H₂O, 0.05% TFA), yielding 2.6 mg (52% yield) of amide SL-1442 as a deep purple film. ¹H NMR (400 MHz, MeOD) δ 7.35 (dd, J=8.2, 2.1 Hz, 1H), 7.28 (d, J=2.0 Hz, 1H), 7.24-7.16 (m, 4H), 7.07 (d, J=2.4 Hz, 1H), 7.04 (d, J=4.6 Hz, 1H), 7.02-6.94 (m, 2H), 6.87 (d, J=8.5 Hz, 1H), 6.74 (d, J=3.9 Hz, 1H), 6.37 (dd, J=3.9, 2.6 Hz, 1H), 6.13 (d, J=4.0 Hz, 1H), 4.26 (s, 2H), 4.03-3.37 (m, 4H), 3.28 (s, 2H), 2.84 (s, 3H), 2.68 (t, J=7.3 Hz, 2H); HRMS (ESI) calc'd for C₃₅H₃₅BClF₂N₈O [M+H]+ 667.2683 found 667.2677.

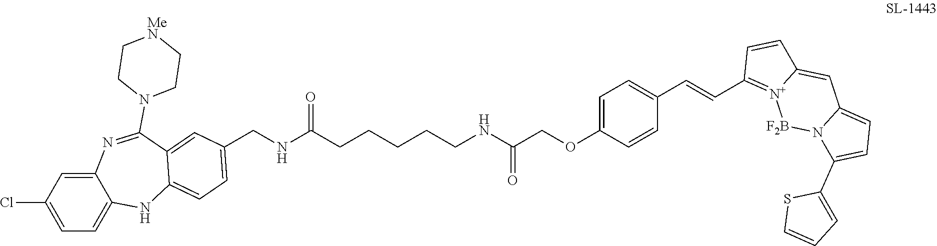

A 25 mL flask, equipped with stir bar, was charged with SL-1441 3.5 mg, 7.5 μmol), BODIPY 630/650 SE (4.9 mg, 7.5 μmol), DIPEA (7 μL, 37 μmol), and DMF (8 mL). The resulting deep purple solution was stirred at 22° C. for 1.5 hours, at which point, HPLC indicated complete consumption of the staring material, and the reaction mixture purified by preparative HPLC (C18, 5→95% MeCN/H₂O, 0.05% TFA) yielding 6 mg (89% yield) of amide SL-1460 as a deep blue-green film. ¹H NMR (400 MHz, MeOD) δ 8.12 (dd, J=3.8, 1.1 Hz, 1H), 7.65-7.56 (m, 3H), 7.55 (s, 1H), 7.51 (d, J=16.3 Hz, 1H), 7.37 (s, 1H), 7.35 (dd, J=8.3, 2.1 Hz, 1H), 7.29 (d, J=2.1 Hz, 1H), 7.26-7.16 (m, 2H), 7.14 (dd, J=7.7, 4.4 Hz, 2H), 7.10 (d, J=2.4 Hz, 1H), 7.03 (td, J=8.8, 2.6 Hz, 4H), 6.88 (d, J=8.5 Hz, 1H), 6.86 (d, J=4.3 Hz, 1H), 4.56 (s, 2H), 4.24 (s, 2H), 3.76 (s, 4H), 3.42 (s, 4H), 3.25 (t, J=6.9 Hz, 2H), 2.93 (s, 3H), 2.20 (t, J=7.3 Hz, 2H), 1.60 (p, J=7.7 Hz, 2H), 1.51 (q, J=7.3 Hz, 2H), 1.26 (tt, J=9.8, 6.0 Hz, 2H); HRMS (ESI) calc'd for C₄₈H₄₉BClF₂N₈O₃S [M+H]⁺ 901.3398 found 901.3386.

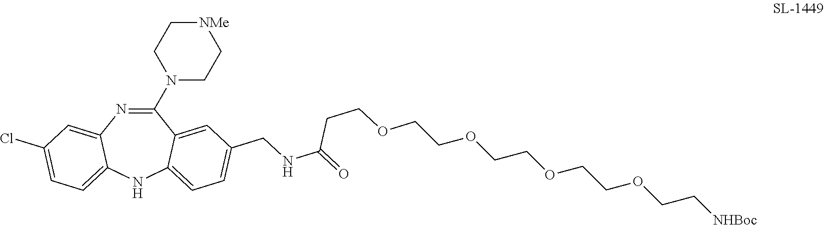 [SMILES: CN1CCN(C2=Nc3cc(Cl)ccc3Nc3ccc(CNC(=O)CCOCCOCCOCCOCCNC(=O)OC(C)(C)C)cc32)CC1]

A 25 mL flask, equipped with stir bar, was charged with SL-1441 (7.0 mg, 15 mmol), 2,2-dimethyl-4-oxo-3,8,11,14,17-pentaoxa-5-azaicosan-20-oic acid (8 mg, 20 μmol), HATU (7 mg, 0.02 mmol), DIPEA (15 μL, 0.10 mmol), and DMF (6 mL). The resulting light yellow solution was stirred at 22° C. for 1.5 hours, at which point, HPLC indicated complete consumption of the staring material, and the solvent removed under reduced pressure. The crude residue was purified by preparative HPLC (C18, 5→95% MeCN/H₂O, 0.05% TFA) yielding 11 mg (quantative) of carbamate SL-1449 as a yellow oil. ¹H NMR (400 MHz, MeOD) δ 7.45 (dd, J=8.3, 2.0 Hz, 1H), 7.36 (d, J=2.0 Hz, 1H), 7.18 (d, J=2.4 Hz, 1H), 7.14-7.03 (m, 2H), 6.94 (d, J=8.6 Hz, 1H), 4.32 (s, 2H), 3.85 (s, 3H), 3.75 (dt, J=15.5, 6.0 Hz, 4H), 3.68-3.43 (m, 26H), 3.21 (dt, J=11.2, 5.6 Hz, 3H), 3.01 (s, 3H), 2.55 (t, J=6.3 Hz, 1H), 2.49 (t, J=5.9 Hz, 2H), 1.43 (d, J=6.8 Hz, 14H); MS (ESI) calc'd for C₃₅H₅₂ClN₆O₇ [M+H]⁺ 703.36 found 703.59.

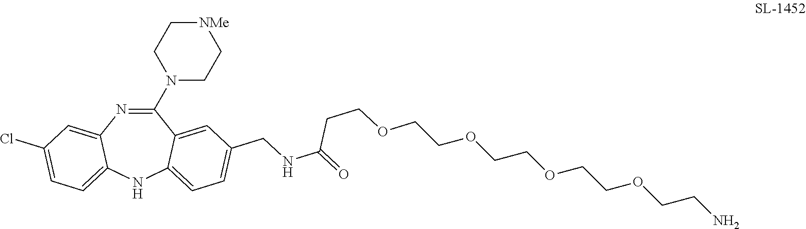 [SMILES: CN1CCN(C2=Nc3cc(Cl)ccc3Nc3ccc(CNC(=O)CCOCCOCCOCCOCCN)cc32)CC1]

A 25 mL flask, equipped with stir bar, was charged with SL-1449 (16 mg, 23 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 16 mg of primary amine SL-1452 as a yellow oil. This material was further used without additional purification.

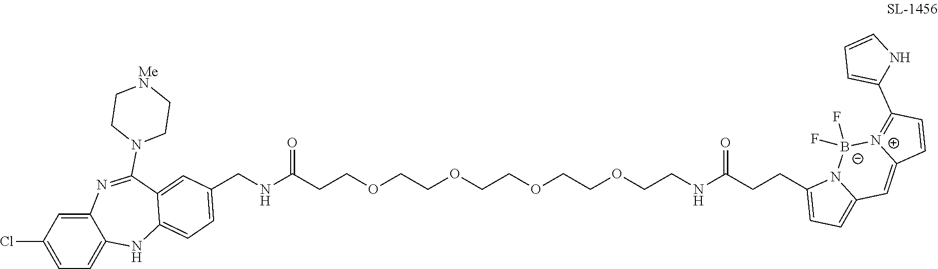 [SMILES: CN1CCN(C2=Nc3cc(Cl)ccc3Nc3ccc(CNC(=O)CCOCCOCCOCCOCCNC(=O)CCc4ccc5n4[B-](F)(F)[N+]4=C(c6ccc[nH]6)C=CC4=C5)cc32)CC1]

A 25 mL flask, equipped with stir bar, was charged with SL-1452 (7 mg, 12 μmol), BODIPY 576/589 SE (5.0 mg, 12 μmol), DIPEA (15 μL, 82 μmol), and DMF (6 mL). The resulting deep purple solution was stirred at 22° C. for 18 hours, at which point, HPLC indicated complete consumption of the staring material, and the reaction mixture purified by preparative HPLC (C18, 5→95% MeCN/H₂O, 0.05% TFA) yielding 2.3 mg (23% yield) of amide SL-1456 as a deep purple film. ¹H NMR (400 MHz, MeOD) δ 7.38 (dd, J=8.2, 2.1 Hz, 1H), 7.29 (d, J=2.0 Hz, 1H), 7.24 (s, 1H), 7.23-7.13 (m, 3H), 7.10 (d, J=2.4 Hz, 1H), 7.06-6.97 (m, 3H), 6.92 (d, J=4.0 Hz, 1H), 6.89 (d, J=8.4 Hz, 1H), 6.54-6.21 (m, 2H), 4.29 (s, 2H), 3.96-3.58 (m, 6H), 3.58-3.45 (m, 15H), 3.43 (s, 3H), 3.36 (t, J=5.4 Hz, 3H), 3.27 (d, J=7.7 Hz, 2H), 2.95 (s, 3H), 2.64 (t, J=7.7 Hz, 2H), 2.45 (t, J=5.8 Hz, 2H); HRMS (ESI) calc'd for C₄₆H₅₆BClF₂N₉O₆ [M+H]⁺ 914.4103 found 914.4089.

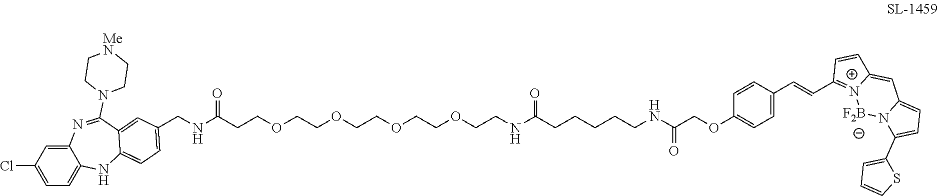

A 25 mL flask, equipped with stir bar, was charged with SL-1452 (7.0 mg, 12 μmol), BODIPY 630/650 SE (7.7 mg, 12 μmol), DIPEA (15 μL, 81 μmol), and DMF (8 mL). The resulting deep purple solution was stirred at 22° C. for 1.5 hour, at which point, HPLC indicated complete consumption of the staring material, and the reaction mixture purified by preparative HPLC (C18, 5→95% MeCN/H₂O, 0.05% TFA) yielding 3.1 mg (23% yield) of amide SL-1459 as a deep blue-green film. ¹H NMR (400 MHz, MeOD) δ 8.12 (dd, J=3.9, 1.1 Hz, 1H), 7.71-7.58 (m, 3H), 7.55 (m, 2H), 7.40 (dd, J=8.3, 2.1 Hz, 1H), 7.38 (s, 1H), 7.31 (d, J=2.0 Hz, 1H), 7.27-7.18 (m, 2H), 7.15 (d, J=2.3 Hz, 2H), 7.14 (s, 1H), 7.10-7.00 (m, 4H), 6.91 (d, J=8.5 Hz, 1H), 6.86 (d, J=4.2 Hz, 1H), 4.57 (s, 2H), 4.29 (s, 2H), 3.74 (t, J=5.8 Hz, 5H), 3.62-3.39 (m, 17H), 3.27 (t, J=5.6 Hz, 3H), 2.97 (s, 3H), 2.46 (t, J=5.8 Hz, 2H), 2.15 (t, J=7.4 Hz, 2H), 1.69-1.45 (m, 4H), 1.37-1.12 (m, 2H); HRMS (ESI) calc'd for C₅₉H₆₉BClF₂N₉O₈S [M+H]⁺ 1148.4818 found 1148.4829.

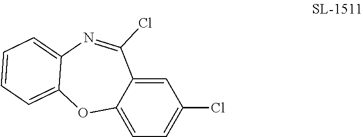 [SMILES: ClC1=Nc2ccccc2Oc2ccc(Cl)cc21]

A 25 mL flask, equipped with stir bar, was charged with 2-chlorodibenzo[b,f][1,4]oxazepin-11(10H)-one (100 mg, 0.4 mmol), N,N-dimethylaniline (0.21 mL, 1.6 mmol), POCl₃ (114 μL, 1.14 mmol), and toluene (4 mL). The resulting suspension was heated to 95° C. for 2.5 hours, and a dark brown solution formed. Solvent was removed under reduced pressure, and the residue dissolved in in a mixture of dioxane (2 mL) and aqueous 2M Na₂CO₃ (3 mL). The resulting solution was heated at 80° C. for 50 minutes, dioxane removed under reduced pressure, and the residue extracted in EtOAc (3×10 mL). Combined EtOAc solutions were dried over MgSO₄, filtered, and the solvent removed under reduced pressure. The residue was purified by silica gel chromatography (0→30% EtOAc/hexanes) to provide 35 mg (33% yield) of imidoyl chloride SL-1511 as a white solid. ¹H NMR (400 MHz, CDCl₃) δ 7.71 (d, J=2.6 Hz, 1H), 7.47 (dd, J=8.7, 2.5 Hz, 1H), 7.33 (dd, J=7.5, 2.1 Hz, 1H), 7.26 (td, J=7.5, 1.7 Hz, 1H, overlap with CHCl3), 7.21 (td, J=7.5, 1.7 Hz, 1H), 7.15 (dd, J=7.6, 1.7 Hz, 1H), 7.13 (d, J=8.7 Hz, 1H); MS (ESI) calc'd for C₁₃H₈Cl₂NO [M+H]⁺ 264.00 found 263.87.

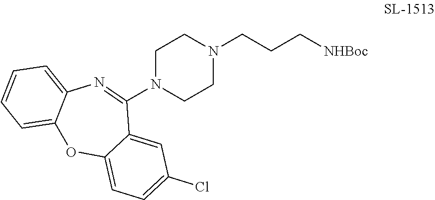 [SMILES: CC(C)(C)OC(=O)NCCCN1CCN(C2=Nc3ccccc3Oc3ccc(Cl)cc32)CC1]

A 10 mL microwave vial, equipped with stir bar, was charged with SL-1511 (35 mg, 0.13 mmol), tert-butyl(3-(piperazin-1-yl)propyl)carbamate (65 mg, 0.27 mmol), K₂CO₃ (46 mg, 0.33 mmol), and dioxane (3 mL). The vial was placed into a microwave reactor and heated to 120° C. for 7 hours. HPLC analysis confirmed consumption of the starting material, and the solution filtered and concentrated under reduced pressure. The crude residue was purified by flash chromatography (gradient elution, 0→20% MeOH/DCM, yielding 32 mg (51% yield) of amidine SL-1513 as a yellow solid. ¹H NMR (400 MHz, MeOD) δ 7.51 (dd, J=8.7, 2.6 Hz, 1H), 7.40 (d, J=2.6 Hz, 1H), 7.29 (d, J=8.7 Hz, 1H), 7.17-7.05 (m, 3H), 7.01 (ddd, J=7.8, 6.7, 2.4 Hz, 1H), 3.53 (br. s, 4H), 3.10 (d, J=6.8 Hz, 2H), 2.62 (br. s, 4H), 2.54-2.40 (m, 2H), 1.72 (p, J=6.9 Hz, 2H), 1.44 (s, 9H); HRMS (ESI) calc'd for C₂₅H₃₂ClN₄O_{3NN} [M+H]⁺ 471.2163 found 471.2152.

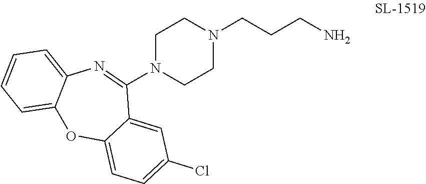 [SMILES: NCCCN1CCN(C2=Nc3ccccc3Oc3ccc(Cl)cc32)CC1]

A 25 mL flask, equipped with stir bar, was charged with SL-1513 (32 mg, 68 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 16 mg of primary amine SL-1452 as a yellow oil. This material was further used without additional purification. MS (ESI) calc'd for C₂₀H₂₄ClN₄O [M+H]⁺ 371.16 found 371.22.

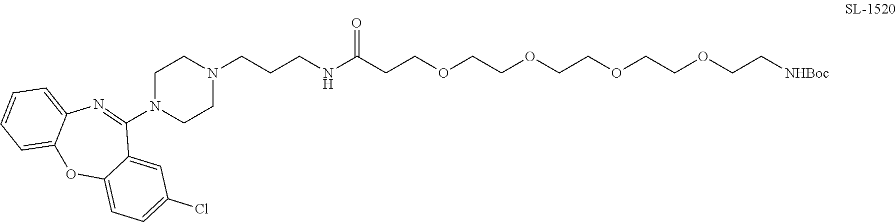 [SMILES: CC(C)(C)OC(=O)NCCOCCOCCOCCOCCC(=O)NCCCN1CCN(C2=Nc3ccccc3Oc3ccc(Cl)cc32)CC1]

A 25 mL flask, equipped with stir bar, was charged with SL-1519 (25 mg, 37 μmol), 2,2-dimethyl-4-oxo-3,8,11,14,17-pentaoxa-5-azaicosan-20-oic acid (31 mg, 84 μmol), HATU (32 mg, 84 μmol), DIPEA (66 μL, 0.47 mmol), and DMF (6 mL). The resulting light yellow solution was stirred at 22° C. for 18 hours, at which point, HPLC indicated complete consumption of the staring material, and the solvent removed under reduced pressure. The crude residue was purified by preparative HPLC (C18, 5→95% MeCN/H₂O, 0.05% TFA) yielding 41 mg (85% yield) of carbamate SL-1520 as a clear oil. ¹H NMR (400 MHz, DMSO-d6) δ 9.58 (br. s, 1H), 8.05 (t, J=5.8 Hz, 1H), 7.68 (dd, J=8.7, 2.6 Hz, 1H), 7.57 (d, J=2.6 Hz, 1H), 7.44 (d, J=8.7 Hz, 1H), 7.23 (dd, J=7.8, 1.5 Hz, 1H), 7.18-6.90 (m, 3H), 6.75 (t, J=5.7 Hz, 1H), 3.61 (t, J=6.4 Hz, 2H), 3.57-3.44 (m, 16H), 3.36 (t, J=6.2 Hz, 2H), 3.26 (s, 2H), 3.17-3.09 (m, 4H), 3.05 (q, J=6.0 Hz, 2H), 2.35 (d, J=6.4 Hz, 2H), 1.97-1.73 (m, 2H), 1.37 (s, 9H); MS (ESI) calc'd for C₃₆H₅₃ClN₅O₈ [M+H]⁺ 718.36 found 718.41.

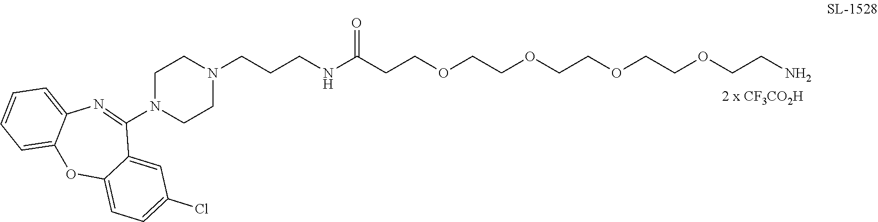 [SMILES: NCCOCCOCCOCCOCCC(=O)NCCCN1CCN(C2=Nc3ccccc3Oc3ccc(Cl)cc32)CC1]

A 25 mL flask, equipped with stir bar, was charged with SL-1520 (41 mg, 57 μmol) and a cleavage cocktail (10 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 35 mg (99% yield) primary amine SL-1528 as a yellow oil. This material was further used without additional purification. MS (ESI) calc'd for C₃₁H₄₅ ClN₅O₆ [M+H]⁺ 618.31 found 618.16.

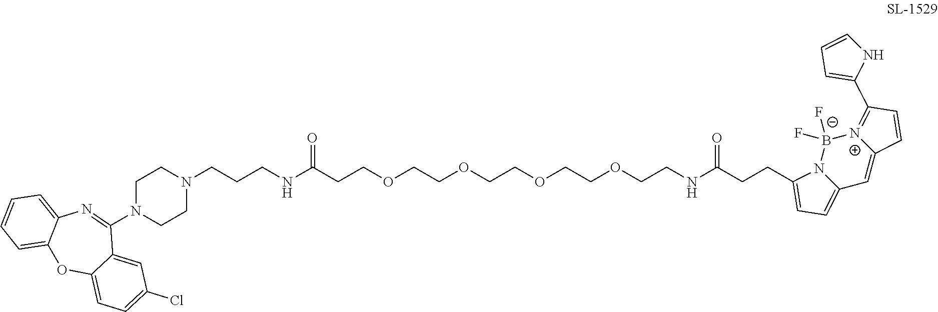

A 25 mL flask, equipped with stir bar, was charged with SL-1528 (35 mg, 56 μmol), BODIPY 576/589 SE (8.0 mg, 19 μmol), DIPEA (50 μL, 0.28 mmol), and DMF (8 mL). The resulting deep purple solution was stirred at 22° C. for 20 hours, at which point, HPLC indicated complete consumption of the staring material, and the reaction mixture purified by preparative HPLC (C18, 5→95% MeCN/H₂O, 0.05% TFA) yielding 16.4 mg (94% yield) of amide SL-1529 as a deep purple film. ¹H NMR (400 MHz, MeOD) δ 10.74 (s, 1H), 7.56 (dd, J=8.7, 2.6 Hz, 1H), 7.47 (d, J=2.6 Hz, 1H), 7.32 (d, J=8.7 Hz, 1H), 7.24 (s, 1H), 7.22-7.18 (m, 3H), 7.18-7.12 (m, 3H), 7.12-7.05 (m, 1H), 6.92 (d, J=3.9 Hz, 1H), 6.42-6.27 (m, 2H), 4.19 (s, 2H), 3.72 (t, J=5.8 Hz, 2H), 3.63-3.55 (m, 14H), 3.52 (t, J=5.5 Hz, 2H), 3.37 (t, J=5.5 Hz, 2H), 3.28 (d, J=7.7 Hz, 2H), 3.17 (t, J=7.2 Hz, 23H), 2.65 (t, J=7.7 Hz, 2H), 2.46 (t, J=5.8 Hz, 2H), 1.92 (q, J=6.8 Hz, 2H); HRMS (ESI) calc'd for C₄₇H₅₇BClF₂N₈O₇ [M+H]⁺ 929.4100 found 929.4094.

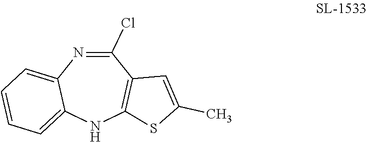 [SMILES: Cc1cc2c(s1)Nc1ccccc1N=C2Cl]

A 25 mL flask, equipped with stir bar, was charged with 2-methyl-5,10-dihydro-4H-benzo[b]thieno[2,3-e][1,4]diazepin-4-one (166 mg, 721 μmol), N,N-dimethylaniline (0.37 mL, 2.9 mmol), POCl₃ (200 μL, 2 mmol), and toluene (8 mL). The resulting suspension was heated to 95° C. for 2.5 hours, and a dark brown solution formed. The solvent was removed under reduced pressure, and the residue dissolved in a mixture of dioxane (4 mL) and aqueous 2M Na₂CO₃ (6 mL). The resulting solution was heated at 80° C. for 50 minutes, dioxane removed under reduced pressure, and the residue extracted in EtOAc (3×25 mL). Combined EtOAc solutions were dried over MgSO₄, filtered, and the solvent removed under reduced pressure. The residue was purified by silica gel chromatography (0→20% EtOAc/hexanes) to provide 7 mg (4% yield) of imidoyl chloride SL-1533 as solid. ¹H NMR (400 MHz, DMSO-d₆) δ 8.22 (s, 1H), 7.03 (td, J=7.6, 1.6 Hz, 1H), 6.92 (td, J=7.6, 1.6 Hz, 1H), 6.80 (dd, J=7.8, 1.5 Hz, 1H), 6.57 (dd, J=7.8, 1.5 Hz, 1H), 6.44 (d, J=1.5 Hz, 1H), 2.21 (d, J=1.3 Hz, 3H); MS (ESI) calc'd for C₁₂H₁₀ClN₂S [M+H]⁺ 249.03 found 249.02.

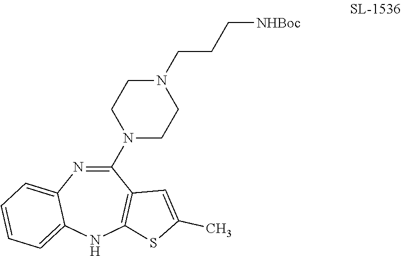 [SMILES: Cc1cc2c(s1)Nc1ccccc1N=C2N1CCN(CCCNC(=O)OC(C)(C)C)CC1]

A 10 mL microwave vial, equipped with stir bar, was charged with SL-1533 (7.0 mg, 28 μmol), tert-butyl (3-(piperazin-1-yl)propyl)carbamate (14 mg, 56 μmol), K₂CO₃ (10 mg, 70 μmol), and dioxane (2 mL). The vial was placed into a microwave reactor and heated to 120° C. for 2 hours. HPLC analysis confirmed consumption of the starting material, and the solution was filtered and concentrated under reduced pressure. The crude residue was purified by preparative HPLC (C18, 5→95% MeCN/H₂O, 0.05% TFA), yielding 5.5 mg (43% yield) of carbamate SL-1536 as a yellow oily solid. ¹H NMR (400 MHz, MeOD) δ 7.29 (td, J=7.6, 1.6 Hz, 1H), 7.25 (dd, J=8.0, 1.6 Hz, 1H), 7.21-7.11 (m, 1H), 6.92 (dd, J=8.0, 1.3 Hz, 1H), 6.62 (q, J=1.3 Hz, 1H), 4.08 (br. s, 4H), 3.74-3.50 (r. s, 4H), 3.29-3.22 (m, 2H), 3.19 (t, J=6.6 Hz, 2H), 2.38 (d, J=1.3 Hz, 3H), 2.07-1.89 (m, 2H), 1.45 (s, 9H); HRMS (ESI) calc'd for C₂₄H₃₄N₅O₂S [M+H]⁺ 456.2433 found 456.2427.

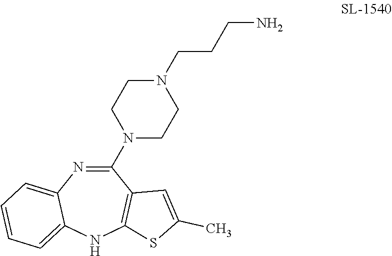 [SMILES: Cc1cc2c(s1)Nc1ccccc1N=C2N1CCN(CCCN)CC1]

A 25 mL flask, equipped with stir bar, was charged with SL-1536 (5.5 mg, 12 μmol) and a cleavage cocktail (7 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 4.2 mg (98% yield) of primary amine SL-1540 as a yellow oil. This material was further used without additional purification. MS (ESI) calc'd for C₁₉H₂₆N₅S [M+H]⁺ 356.19 found 356.08.

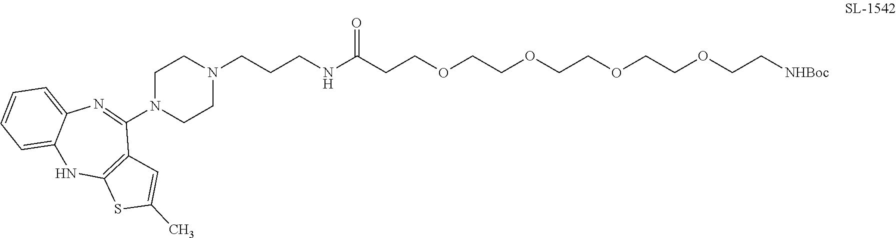 [SMILES: Cc1cc2c(s1)Nc1ccccc1N=C2N1CCN(CCCNC(=O)CCOCCOCCOCCOCCNC(=O)OC(C)(C)C)CC1]

A 25 mL flask, equipped with stir bar, was charged with SL-1540 (4.2 mg, 12 mmol), 2,2-dimethyl-4-oxo-3,8,11,14,17-pentaoxa-5-azaicosan-20-oic acid (5.4 mg, 15 μmol), HATU (5.6 mg, 15 μmol), DIPEA (16 μL, 11 μmmol), and DMF (6 mL). The resulting light yellow solution was stirred at 22° C. for 3 hours, at which point, HPLC indicated complete consumption of the staring material, and the solvent removed under reduced pressure. The crude residue was purified by preparative HPLC (C18, 5→95% MeCN/H₂O, 0.05% TFA) yielding 9 mg (quantative) of carbamate SL-1542 as a yellow oil. ¹H NMR (400 MHz, MeOD) δ 7.39-7.27 (m, 1H), 7.25 (dd, J=8.0, 1.5 Hz, 1H), 7.22-7.07 (m, 1H), 6.93 (dd, J=8.0, 1.3 Hz, 1H), 6.63 (q, J=1.2 Hz, 1H), 4.10 (br. s, 4H), 3.77 (t, J=5.9 Hz, 2H), 3.62-3.59 (m, 16H), 3.56 (q, J=5.5 Hz, 4H), 3.40 (dd, J=7.0, 5.6 Hz, 2H), 3.25 (d, J=7.1 Hz, 2H), 2.51 (t, J=5.8 Hz, 2H), 2.39 (d, J=1.2 Hz, 3H), 2.09-1.95 (m, 2H), 1.43 (s, 9H); HRMS (ESI) calc'd for C₃₅H₅₄N₆O₇SNa [M+Na]⁺ 725.3672 found 725.3661.

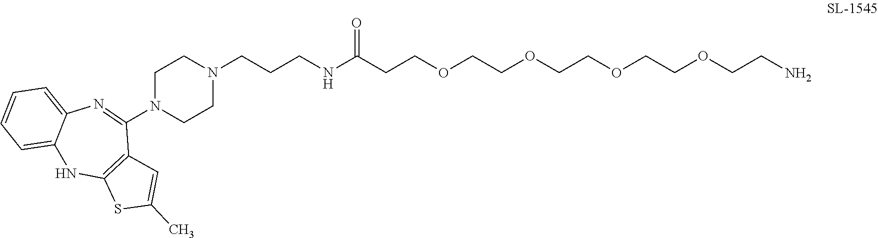 [SMILES: Cc1cc2c(s1)Nc1ccccc1N=C2N1CCN(CCCNC(=O)CCOCCOCCOCCOCCN)CC1]

A 25 mL flask, equipped with stir bar, was charged with SL-1542 (8 mg, 12 μmol) and a cleavage cocktail (7 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and the solvent removed under reduced pressure to provide 6 mg (85% yield) primary amine SL-1528 as a yellow oil. This material was further used without additional purification. MS (ESI) calc'd for C₃₀H₄₇N₆O₅S [M+H]⁺ 603.33 found 603.08.

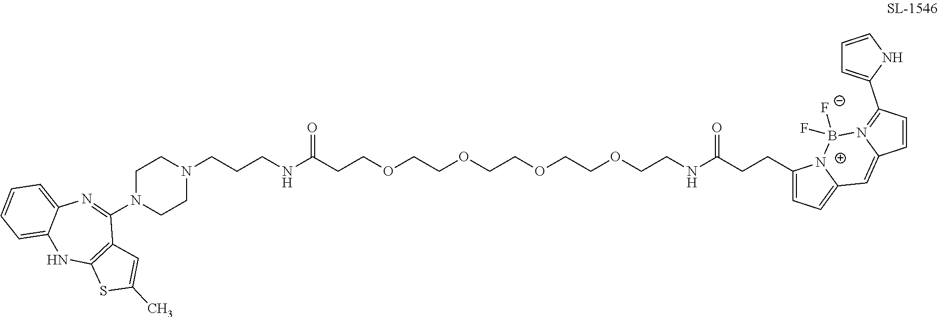

A 25 mL flask, equipped with stir bar, was charged with SL-1545 (6 mg, 10 μmol), BODIPY 576/589 SE (3.5 mg, 8 μmol), DIPEA (14 μL, 82 μmol), and DMF (8 mL). The resulting deep purple solution was stirred at 22° C. for 3 hours, at which point, HPLC indicated complete consumption of the staring material, and the reaction mixture purified by preparative HPLC (C18, 5→95% MeCN/H₂O, 0.05% TFA) yielding 2 mg (27% yield) of amide SL-1516 as a deep purple film. ¹H NMR (400 MHz, MeOD) δ 7.28 (ddd, J=8.0, 7.2, 1.7 Hz, 1H), 7.25 (s, 1H), 7.24-7.18 (m, 4H), 7.15 (ddd, J=8.0, 7.2, 1.3 Hz, 1H), 7.01 (d, J=4.6 Hz, 1H), 6.96-6.85 (m, 2H), 6.55 (q, J=1.2 Hz, 1H), 6.34 (td, J=4.2, 1.8 Hz, 2H), 4.03 (s, 4H), 3.74 (t, J=5.8 Hz, 2H), 3.59 (d, J=3.7 Hz, 12H), 3.53 (t, J=5.6 Hz, 2H), 3.46 (br. s, 4H), 3.41-3.34 (m, 4H), 3.27 (d, J=7.6 Hz, 2H), 3.18 (t, J=7.0 Hz, 2H), 2.64 (t, J=7.6 Hz, 2H), 2.48 (t, J=5.7 Hz, 2H), 2.34 (d, J=1.2 Hz, 3H), 1.95 (p, J=6.8 Hz, 2H); MS (ESI) calc'd for C₄₆H₅₉BF₂N₉O₆S [M+H]⁺ 914.44 found 914.26.

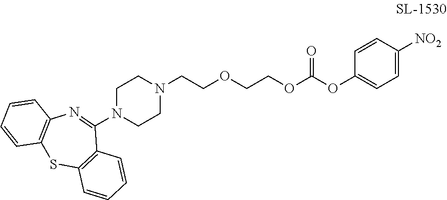 [SMILES: O=C(OCCOCCN1CCN(C2=Nc3ccccc3Sc3ccccc32)CC1)Oc1ccc([N+](=O)[O-])cc1]

A 50 mL flask, equipped with stir bar, was charged with quetiapine (263 mg, 686 μmol), 4-nitrophenyl chloroformate (200 mg, 1 mmol), and DCM (30 mL). The resulting solution was cooled to 0° C. under Ar and pyridine (166 μL, 2.06 mmol) was added dropwise. The solution was allowed to warm up to 22° C. and left stirred for 20 hours, at which point, solvent removed under reduced pressure and residue purified by silica gel chromatography (0→50% MeOH/DCM) to provide 121 mg (32% yield) of carbonate SL-1530 as a yellow oily solid. ¹H NMR (400 MHz, CDCl₃) δ 8.35-8.10 (m, 2H), 7.51 (dt, J=7.3, 1.2 Hz, 1H), 7.44-7.35 (m, 3H), 7.35-7.27 (m, 3H), 7.18 (t, J=7.7 Hz, 1H), 7.06 (dd, J=8.0, 1.5 Hz, 1H), 6.99-6.78 (m, 1H), 4.52-4.35 (m, 2H), 3.85-3.75 (m, 2H), 3.70 (s, 2H), 3.37 (d, J=151.4 Hz, 4H), 2.76-2.38 (m, 5H); MS (ESI) calc'd for C₂₈H₂₉N₄O₆S [M+H]⁺ 549.18 found 549.03.

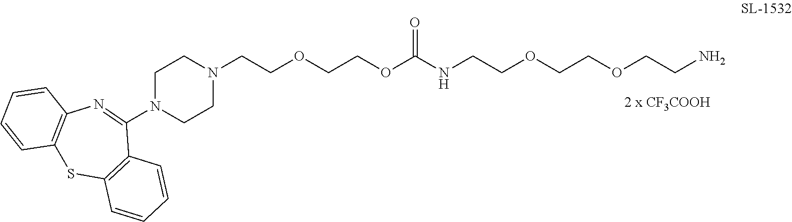 [SMILES: NCCOCCOCCNC(=O)OCCOCCN1CCN(C2=Nc3ccccc3Sc3ccccc32)CC1]

A 250 mL flask, equipped with stir bar, was charged with SL-1530 (60 mg, 110 μmol), 2,2′-(ethane-1,2-diylbis(oxy))bis(ethan-1-amine) (81 mg, 0.55 mmol), DIPEA (180 μL, 1.0 mmol), and DMF (100 mL). The resulting yellow solution was stirred for 18 hours at 22° C., at which point, HPLC indicated complete consumption of the staring material, and solvent removed under reduced pressure, and the residue was purified by preparative HPLC (C18, 5→95% MeCN/H₂O, 0.05% TFA), yielding 86 mg (quantative) of amide SL-1532 as a deep purple film. ¹H NMR (400 MHz, MeOD) δ 7.71 (dd, J=7.8, 1.3 Hz, 1H), 7.68-7.46 (m, 4H), 7.42-7.28 (m, 2H), 7.21 (ddd, J=7.7, 6.8, 2.1 Hz, 1H), 4.22 (t, J=4.6 Hz, 2H), 4.17-3.79 (m, 6H), 3.79-3.62 (m, 10H), 3.57 (d, J=9.9 Hz, 2H), 3.52 (t, J=5.7 Hz, 2H), 3.51-3.40 (m, 2H), 3.27 (t, J=5.7 Hz, 2H), 3.12 (t, J=5.1 Hz, 2H); HRMS (ESI) calc'd for C₂₇H₃₉N₅O₅S [M+H]⁺ 558.2750 found 558.2746.

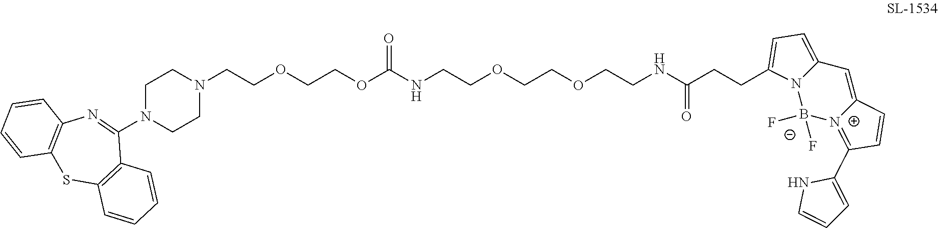

A 25 mL flask, equipped with stir bar, was charged with SL-1532 (8 mg, 10 μmol), BODIPY 576/589 SE (4 mg, 9 μmol), DIPEA (16 μL, 94 μmol), and DMF (8 mL). The resulting deep purple solution was stirred at 22° C. for 16 hours, at which point, HPLC indicated complete consumption of the staring material, and the reaction mixture purified by preparative HPLC (C18, 5→95% MeCN/H₂O, 0.05% TFA) yielding 5.1 mg (63% yield) of amide SL-1534 as a deep purple film. ¹H NMR (400 MHz, MeOD) δ δ 7.57 (dd, J=7.7, 1.2 Hz, 1H), 7.51-7.35 (m, 4H), 7.28-7.22 (m, 2H), 7.22-7.16 (m, 3H), 7.09 (dd, J=8.0, 1.4 Hz, 1H), 7.04-6.87 (m, 3H), 6.39-6.22 (m, 2H), 4.20 (t, J=4.6 Hz, 2H), 3.83-3.78 (m, 2H), 3.69 (t, J=4.5 Hz, 3H), 3.62-3.42 (m, 12H), 3.42-3.35 (m, 6H), 3.29-3.16 (m, 4H), 2.66 (t, J=7.7 Hz, 2H); HRMS (ESI) calc'd for C₄₄H₅₂BF₂N₈O₆S [M+H]⁺ 869.3792 found 869.3784.

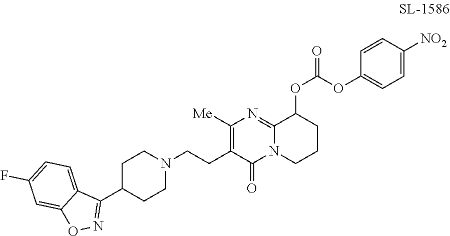 [SMILES: Cc1nc2n(c(=O)c1CCN1CCC(c3noc4cc(F)ccc34)CC1)CCCC2OC(=O)Oc1ccc([N+](=O)[O-])cc1]

A 50 mL flask, equipped with stir bar, was charged with paliperidone (220 mg, 516 μmol), pyridine (1 mL), and DCM (10 mL). To the resulting solution, 4-nitrophenyl chloroformate (200 mg, 1 mmol) was slowly added. The solution was stirred at 22° C. for 20 hours, at which point, the solution was purified by silica gel chromatography (0→50% MeOH/DCM) to provide carbonate SL-1586 as a yellow solid. MS (ESI) calc'd for C₃₀H₃₁FN₅O₇ [M+H]⁺ 592.22 found 592.11.

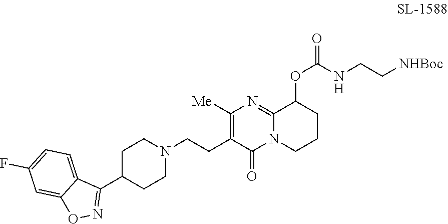 [SMILES: Cc1nc2n(c(=O)c1CCN1CCC(c3noc4cc(F)ccc34)CC1)CCCC2OC(=O)NCCNC(=O)OC(C)(C)C]

A 25 mL flask, equipped with stir bar, was charged with SL-1586 (19 mg, 32 μmol), tert-butyl (2-aminoethyl)carbamate (6.2 mg, 39 μmol), DIPEA (70 μL, 97 μmol), and MeCN (10 mL). The resulting yellow solution was stirred for 2 hours at 22° C., at which point, HPLC indicated complete consumption of the staring material, and solvent removed under reduced pressure, and the residue purified by preparative HPLC (C18, 5→95% MeCN/H₂O, 0.05% TFA) yielding 12 mg (60% yield) of carbamate SL-1588 as a clear oil. ¹H NMR (400 MHz, MeOD) δ 7.92 (dd, J=8.8, 5.1 Hz, 1H), 7.45 (dd, J=8.7, 2.2 Hz, 1H), 7.22 (td, J=9.0, 2.2 Hz, 1H), 5.72-5.53 (m, 1H), 4.07 (dt, J=14.3, 5.1 Hz, 1H), 3.96-3.77 (m, 3H), 3.75 3.38 (m, 2H), 3.23-3.10 (m, 4H), 3.10-2.89 (m, 2H), 2.63-2.33 (m, 6H), 2.33-1.91 (m, 6H), 1.43 (s, 9H).

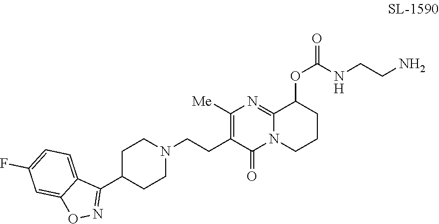 [SMILES: Cc1nc2n(c(=O)c1CCN1CCC(c3noc4cc(F)ccc34)CC1)CCCC2OC(=O)NCCN]

A 25 mL flask, equipped with stir bar, was charged with carbamate SL-1590 (12 mg, 19 μmol) and a cleavage cocktail (7 mL, 80:20:1 DCM/TFA/TIPS). The resulting light yellow solution was stirred at 22° C. for 1.5 hours, at which point, HPLC indicated complete consumption of the staring material, and the solvent removed under reduced pressure. The residue was dissolved in 10 mL MeOH, and solvent removed under reduced pressure to provide 10 mg (quantative) primary amine SL-1590 as a yellow oil, which was further used without additional purification. MS (ESI) calc'd for C₂₆H₃₄FN₆O₄ [M+H]⁺ 513.26 found 513.13.

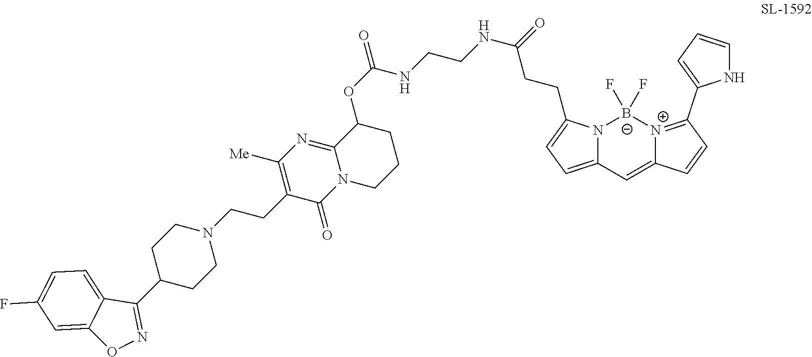 [SMILES: Cc1nc2n(c(=O)c1CCN1CCC(c3noc4cc(F)ccc34)CC1)CCCC2OC(=O)NCCNC(=O)CCc1ccc2n1[B-](F)(F)[N+]1=C(c3ccc[nH]3)C=CC1=C2]

A 25 mL flask, equipped with stir bar, was charged with SL-1590 (9 mg, 18 μmol), BODIPY 576/589 SE (5 mg, 12 μmol), DIPEA (15 μL, 82 μmol), and DMF (7 mL). The resulting deep purple solution was stirred at 22° C. for 2 hours, at which point, HPLC indicated complete consumption of the staring material, and the reaction mixture purified by preparative HPLC (C18, 5→95% MeCN/H₂O, 0.05% TFA) yielding 3.2 mg (33% yield) of amide SL-1592 as a deep purple film. ¹H NMR (400 MHz, MeOD) δ 7.88 (dd, J=8.8, 5.1 Hz, 1H), 7.44 (dd, J=8.7, 2.2 Hz, 1H), 7.24 (d, J=3.9 Hz, 1H), 7.20 (t, J=4.2 Hz, 4H), 7.09-6.95 (m, 1H), 6.92 (d, J=3.9 Hz, 1H), 6.52-6.15 (m, 2H), 5.62 (d, J=5.6 Hz, 1H), 4.03 (dd, J=14.5, 5.7 Hz, 1H), 3.83 (t, J=13.4 Hz, 3H), 3.49 (d, J=24.6 Hz, 1H), 3.42-3.32 (m, 2H), 3.26-3.06 (m, 5H), 3.06-2.78 (m, 2H), 2.69-2.49 (m, 2H), 2.42 (t, J=15.6 Hz, 2H), 2.32 (m, 3H), 2.24-1.85 (m, 5H); HRMS (ESI) calc'd for C₄₂H₄₆BF₃N₉O₅ [M+H]⁺ 824.3667 found 824.3677.

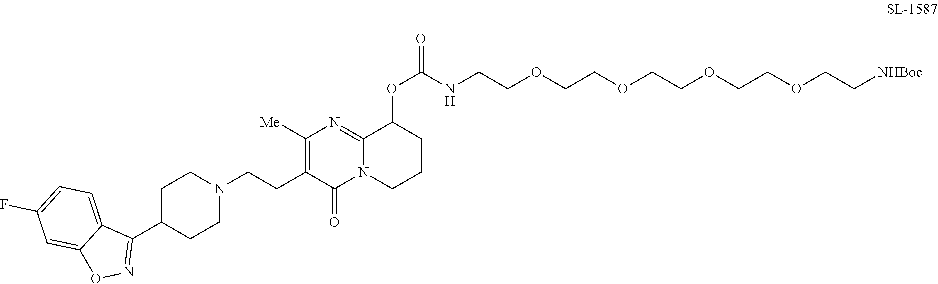 [SMILES: Cc1nc2n(c(=O)c1CCN1CCC(c3noc4cc(F)ccc34)CC1)CCCC2OC(=O)NCCOCCOCCOCCOCCNC(=O)OC(C)(C)C]

A 25 mL flask, equipped with stir bar, was charged with SL-1586 (19 mg, 32 μmol), tert-butyl (14-amino-3,6,9,12-tetraoxatetradecyl)carbamate (13 mg, 39 μmol), DIPEA (17 μL, 97 μmol), and MeCN (10 mL). The resulting yellow solution was stirred for 1 hour at 22° C., at which point, HPLC indicated complete consumption of the staring material, solvent removed under reduced pressure, and the residue purified by preparative HPLC (C18, 5→95% MeCN/H₂O, 0.05% TFA) yielding 13 mg (51% yield) of carbamate SL-1587 as a clear oil. ¹H NMR (400 MHz, MeOD) δ 8.01-7.75 (m, 1H), 7.45 (dd, J=8.8, 2.2 Hz, 1H), 7.22 (td, J=9.0, 2.2 Hz, 1H), 5.63 (t, J=4.6 Hz, 1H), 4.17-4.00 (m, 1H), 4.00-3.79 (m, 3H), 3.79-3.52 (m, 16H), 3.50 (t, J=5.7 Hz, 3H), 3.26-3.14 (m, 3H), 3.11-2.85 (m, 2H), 2.56-2.42 (m, 2H), 2.38 (d, J=10.8 Hz, 3H), 2.31-2.15 (m, 2H), 2.15-1.90 (m, 4H), 1.43 (s, 9H); MS (ESI) calc'd for C₃₉H₅₈FN₆O₁₀ [M+H]⁺ 789.42 found 789.29.

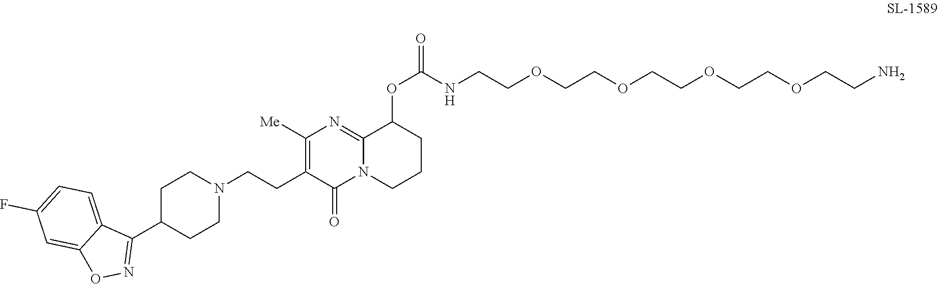 [SMILES: Cc1nc2n(c(=O)c1CCN1CCC(c3noc4cc(F)ccc34)CC1)CCCC2OC(=O)NCCOCCOCCOCCOCCN]

A 25 mL flask, equipped with stir bar, was charged with carbamate SL-1587 (13 mg, 16 μmol) and a cleavage cocktail (7 mL, 80:20:1 DCM/TFA/TIPS). The resulting light yellow solution was stirred at 22° C. for 1 hour, at which point, HPLC indicated complete consumption of the staring material, and the solvent removed under reduced pressure. The residue was dissolved in 10 mL MeOH, and solvent removed under reduced pressure to provide 12 mg (quantative) primary amine SL-1589 as a yellow oil, which was further used without additional purification. MS (ESI) calc'd for C₃₄H₅₀FN₆O₈ [M+H]⁺ 689.37 found 689.34.

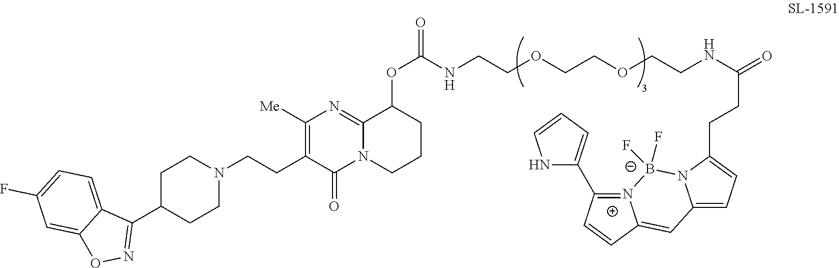

A 25 mL flask, equipped with stir bar, was charged with SL-1589 (12 mg, 17 μmol), BODIPY 576/589 SE (6 mg, 14 μmol), DIPEA (18 μL, 99 μmol), and DMF (7 mL). The resulting deep purple solution was stirred at 22° C. for 2.5 hours, at which point, HPLC indicated complete consumption of the staring material, and the reaction mixture purified by preparative HPLC (C18, 5→95% MeCN/H₂O, 0.05% TFA) yielding 3.6 mg (26% yield) of amide SL-1591 as a deep purple film. ¹H NMR (400 MHz, MeOD) δ 7.86 (dd, J=8.8, 5.0 Hz, 1H), 7.52-7.37 (m, 1H), 7.29-7.09 (m, 5H), 7.00 (d, J=4.6 Hz, 1H), 6.92 (d, J=4.0 Hz, 1H), 6.40-6.15 (m, 2H), 5.63 (d, J=3.9 Hz, 1H), 4.05 (dd, J=14.3, 4.8 Hz, 1H), 3.85 (t, J=15.4 Hz, 3H), 3.53 (td, J=5.4, 2.2 Hz, 5H), 3.37 (td, J=5.2, 2.6 Hz, 3H), 3.28-3.07 (m, 6H), 2.97 (dq, J=7.8, 3.9 Hz, 2H), 2.64 (t, J=7.7 Hz, 2H), 2.43 (d, J=14.9 Hz, 2H), 2.32 (d, J=12.1 Hz, 3H), 2.24-1.94 (m, 5H); HRMS (ESI) calc'd for C₅₀H₆₂₆BF₃N₉O₉ [M+H]⁺ 1001.4773 found 1000.4716.

### Synthesis of Amitriptyline Fluorescent Tracers:

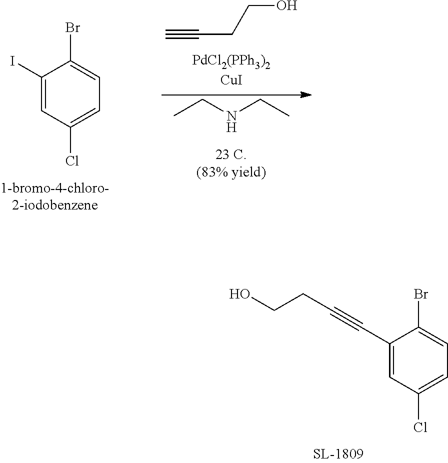

A 50 mL round bottom flask, equipped with stir bar and rubber septum under argon atmosphere, was charged with 1-bromo-4-chloro-2-iodobenzene (3.17 g, 10 mmol), CuI (38 mg, 0.2 mmol), and PdCl₂(PPh₃)₂ (140 mg, 0.2 mmol). Degassed diethylamine (18 mL) was added via syringe followed by 3-butyn-1-ol. The reaction mixture was stirred at 23° C. for 72 hours at which point HPLC indicated complete consumption of the staring material, and the solvent removed under reduced pressure. The crude residue was purified by flash chromatography (gradient elution, 0→50% EtOAc/heptane, yielding 2.15 g (83% yield) of alkyne SL-1809 as a yellow solid. ¹H NMR (400 MHz, CDCl3) δ 7.49 (d, J=8.6 Hz, 1H), 7.43 (d, J=2.6 Hz, 1H), 7.13 (dd, J=8.6, 2.5 Hz, 1H), 3.85 (t, J=6.1 Hz, 2H), 2.74 (t, J=6.1 Hz, 2H); ¹³C NMR (100 MHz, CDCl3) δ 133.3, 133.0, 132.9, 129.3, 126.8, 123.6, 93.1, 80.4, 77.3, 77.0, 76.7, 60.9, 24.0; HRMS (ESI) calc'd for C₁₀H₉BrClO [M+H]⁺ 258.9525 found 258.9520.

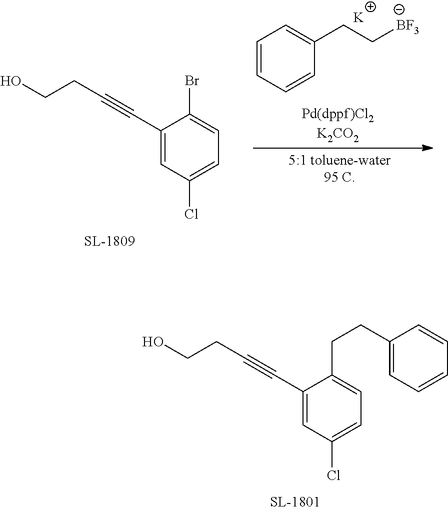

A 50 mL pressure vessel, equipped with stir bar, was charged with SL-1809 (146 mg, 560 μmol), potassium trifluoro(phenethyl)borate (125 mg, 590 μmol), K₂CO₃ (206 mg, 1.69 mmol), Pd(dppf)Cl₂ (8 mg, 11 μmol). Headspace was flushed with argon and degassed toluene (5 mL) and degassed water (1 mL) were added. The reaction mixture was stirred at 95° C. for 21 hours. The reaction mixture was cooled to ambient temperature, diluted with EtOAc (40 mL), and dried with MgSO₄, filtered, and the solvent removed under reduced pressure. The crude residue was purified by preparative HPLC (C18, 5→95% MeCN/H₂O, 0.05% TFA) yielding 60 mg (38% yield) of alkyne SL-1801 as a clear oil. ¹H NMR (400 MHz, CDCl3) δ 7.39 (d, J=2.3 Hz, 1H), 7.33-7.26 (m, 2H), 7.24-7.19 (m, 1H), 7.16 (dd, J=8.4, 2.0 Hz, 3H), 7.04 (d, J=8.2 Hz, 1H), 3.83 (t, J=6.3 Hz, 2H), 3.13-2.95 (m, 2H), 2.90 (dd, J=9.7, 6.2 Hz, 2H), 2.74 (t, J=6.3 Hz, 2H); ¹³C NMR (100 MHz, CDCl3) δ 13C NMR (101 MHz, CDCl3) δ 142.0, 141.4, 132.0, 131.4, 130.1, 128.4, 128.4, 128.1, 126.0, 124.4, 91.2, 79.8, 61.2, 36.8, 36.2, 23.9; HRMS (ESI) calc'd for C₁₈H₁₈ClO [M+H]⁺ 285.1046 found 285.1044.

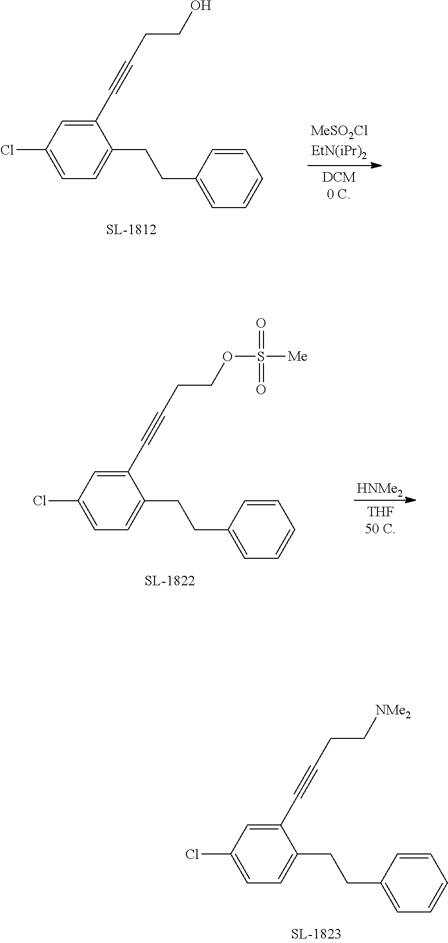 [SMILES: OCCC#Cc1cc(Cl)ccc1CCc1ccccc1 ; CS(=O)(=O)OCCC#Cc1cc(Cl)ccc1CCc1ccccc1 ; CN(C)CCC#Cc1cc(Cl)ccc1CCc1ccccc1 ; "HNMe2"]

A 25 mL round bottom flask, equipped with stir bar, was charged with SL-1812 (72 mg, 0.25 mmol), EtN(iPr)₂ (90 μL, 0.51 mmol), and DCM (7 mL). The resulting solution was cooled to 0° C. under argon followed by addition of mesyl chloride (29 μL, 0.38 mmol). The reaction mixture was stirred at 0° C. for 1 hour at which point HPLC indicated complete consumption of the staring material, and the solvent removed under reduced pressure. The crude residue was used without additional purification in the next step.

A 25 mL round bottom flask, equipped with stir bar, was charged with SL-1822 (90 mg, 0.25 mmol) and 2M dietylamine solution in THF (12 mL, 25 mmol). The reaction mixture was stirred at 50° C. for 20 hours at which point HPLC indicated complete consumption of the staring material, and the solvent removed under reduced pressure. The crude residue was purified by flash chromatography (gradient elution, 0→100% EtOAc/heptane, yielding 26 mg (35% yield) of amine SL-1823 as a clear oil. ¹H NMR (400 MHz, CDCl3) δ 7.37 (d, J=2.3 Hz, 1H), 7.28 (dd, J=8.0, 6.6 Hz, 2H), 7.23-7.16 (m, 3H), 7.14 (dd, J=8.2, 2.3 Hz, 1H), 7.02 (d, J=8.2 Hz, 1H), 3.07-2.95 (m, 2H), 2.95-2.81 (m, 2H), 2.64 (s, 4H), 2.31 (s, 6H); ¹³C NMR (100 MHz, CDCl3) δ 13C NMR (101 MHz, CDCl3) δ 142.0, 141.6, 131.9, 131.3, 130.0, 128.4, 128.3, 127.9, 126.0, 124.8, 78.8, 77.3, 77.0, 76.7, 58.3, 45.1, 36.7, 36.2, 18.5; HRMS (ESI) calc'd for C₂₀H₂₃ClN [M+H]⁺ 312.1519 found 312.1516.

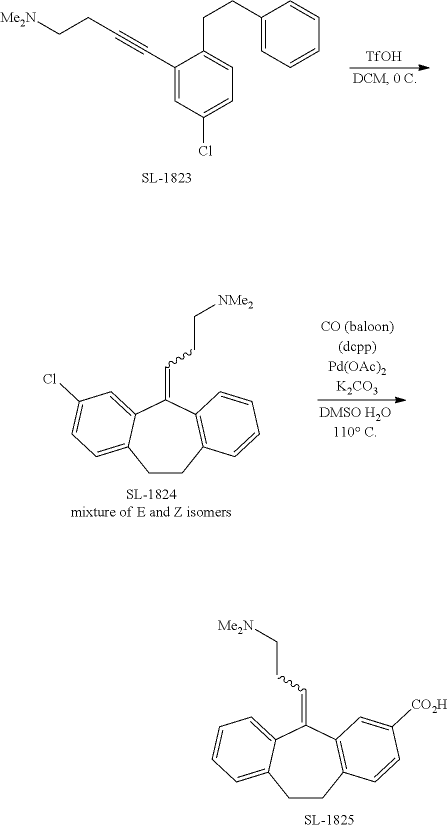

A 10 mL round bottom flask, equipped with stir bar, was charged with a solution of SL-1823 (25 mg, 80 μmol) in DCM (3 mL). The solution was cooled to 0° C., and triflic acid (39 μL, 0.44 mmol) was added in one portion. The resulting brown solution is stirred at 0° C. for 10 minutes at which point the reaction was quenched by addition of saturated aqueous solution of K₂CO₃ (3 mL). Organic layer was separated and aqueous solution was extracted (2×3 mL DCM). Organics were combined, dried over MgSO4, filtered and concentrated in vacuo. The crude residue was used in the next step without further purification. 41 NMR (400 MHz, CDCl3, reported for mixture of E/Z isomers) δ 7.25-6.85 (m, 7H), 5.86-5.80 (m, 1H), 3.41-3.18 (m, 2H), 3.00-2.86 (m, 1H), 2.81-2.64 (m, 3H), 2.44 (s, 8H); MS (ESI) calc'd for C₁₀H₂₃ClN [M+H]⁺ 312.15 found 312.11. Single peak on HPLC at 254 nm.

A 50 mL round bottom flask, equipped with stir bar and septum was charged with SL-1824 (25 mg, 80 μmol), K₂CO₃ (33 mg, 0.24 mmol), Pd(OAc)₂ (1.8 mg, 8.0 μmol), and [dcpp 2BF₄] (9.8 mg, 16 μmol). Flask was evacuated and backfilled with argon (3× times repeated). Degassed DMSO (2 mL) and H₂O (0.2 mL) were added, and the reaction vessel was evacuated and backfilled with carbon monoxide (3× times repeated). CO was allowed to bubble through the solution for 5 minutes. The resulting yellow suspension was heated to 110° C. under CO balloon for 18 hours at which point HPLC analysis indicated complete consumption of the starting material. The reaction mixture was diluted with MeOH (3 mL), passed through syringe filter, and purified by preparative HPLC (C18, 5→95% MeCN/H₂O, 0.05% TFA) yielding 25 mg (97% yield) of E/Z mixture of carboxylic acids SL-1825 as a clear oil. ¹H NMR (400 MHz, MeOD, reported for mixture of E/Z isomers) δ 8.06-7.67 (m, 2H), 7.49-6.92 (m, 5H), 5.89 (m, 1H), 3.49-3.34 (m, 2H), 3.25 (m, 2H), 3.09-2.90 (m, 1H), 2.81 (d, J=12.1 Hz, 7H), 2.66-2.40 (m, 2H); MS (ESI) calc'd for C₂₁H₂₄NO₂ [M+H]⁺ 322.18 found 322.15.

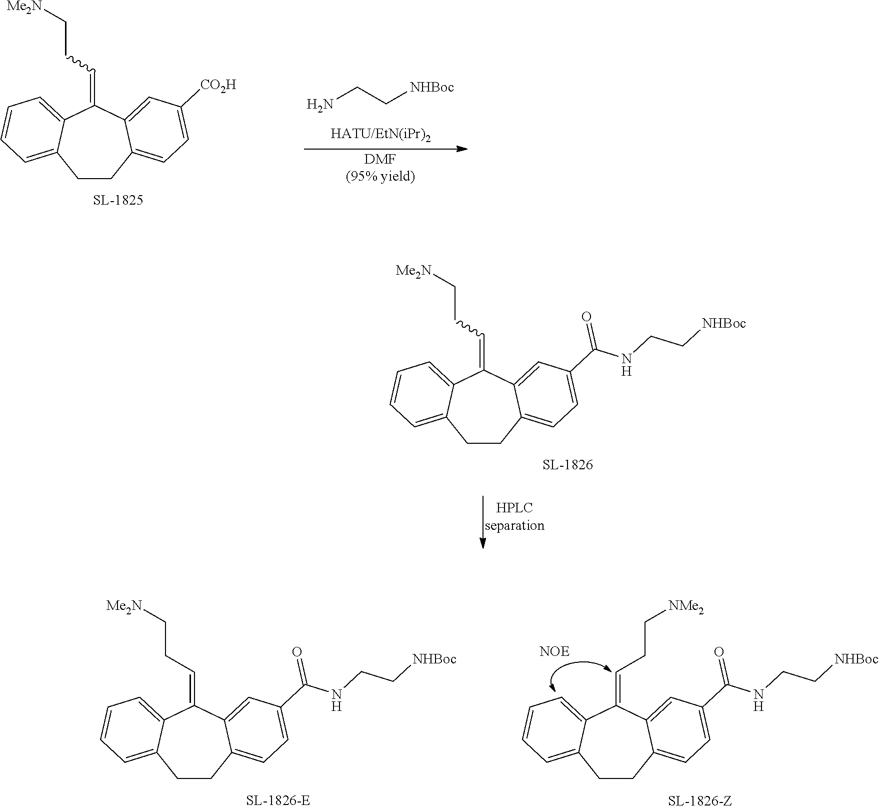 [SMILES: CN(C)CCC=C1c2ccccc2CCc2ccc(C(=O)O)cc21 ; CC(C)(C)OC(=O)NCCN ; CN(C)CCC=C1c2ccccc2CCc2ccc(C(=O)NCCNC(=O)OC(C)(C)C)cc21 ; CN(C)CC/C=C1\c2ccccc2CCc2ccc(C(=O)NCCNC(=O)OC(C)(C)C)cc21 ; CN(C)CC/C=C1/c2ccccc2CCc2ccc(C(=O)NCCNC(=O)OC(C)(C)C)cc21]

A 25 mL flask, equipped with stir bar, was charged with SL-1825 (25 mg, 78 μmol), tert-butyl (2-aminoethyl) carbamate (37 mg, 97 μmol), HATU (16 mg, 97 μmol), EtN(iPr)₂ (70 μL, 0.39 mmol), and DMF (8 mL). The resulting light-yellow solution was stirred at 22° C. for 2.5 hours at which point HPLC indicated complete consumption of the staring material, and the solvent was removed under reduced pressure. The crude residue was purified by preparative HPLC (C18, 5→95% MeCN/H₂O, 0.05% TFA) yielding 10.6 mg (29% yield) of E-isomer of amide SL-1826-E as a clear oil and 4.7 mg (13% yield) of Z-isomer of amide SL-1826-Z al a clear oil (42% combined yield).

SL-1826-E: ¹H NMR (400 MHz, MeOD) δ 7.80 (d, J=1.9 Hz, 1H), 7.62 (dd, J=8.0, 2.0 Hz, 1H), 7.34-7.20 (m, 3H), 7.20-7.12 (m, 2H), 5.92 (t, J=7.3 Hz, 1H), 3.58-3.41 (m, 3H), 3.41-3.34 (m, 2H), 3.28-3.15 (m, 4H), 3.00 (m, 1H), 2.88-2.75 (m, 7H), 2.59 (p, J=8.3, 7.6 Hz, 2H), 1.41 (s, 9H); MS (ESI) calc'd for C₂₈H₃₈N₃O₃ [M+H]⁺ 464.3 found 464.4.

SL-1826-Z: ¹H NMR (400 MHz, MeOD) δ 7.82-7.69 (m, 1H), 7.63 (d, J=1.9 Hz, 1H), 7.40 (d, J=7.9 Hz, 1H), 7.31 (dd, J=6.8, 2.4 Hz, 1H), 7.19 (m, 2H), 7.10 (dd, J=7.2, 1.8 Hz, 1H), 5.89 (t, J=7.4 Hz, 1H), 3.45 (t, J=6.0 Hz, 3H), 3.39 (s, 1H), 3.31-3.17 (m, 3H), 2.93 (d, J=13.3 Hz, 2H), 2.88-2.71 (m, 6H), 2.71-2.31 (m, 2H), 1.41 (s, 9H); MS (ESI) calc'd for C₂₈H₃₈N₃O₃ [M+H]⁺ 464.3 found 464.4.

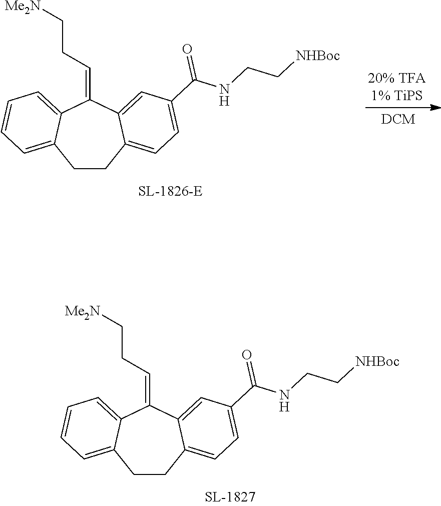 [SMILES: CN(C)CC/C=C1\c2ccccc2CCc2ccc(C(=O)NCCNC(=O)OC(C)(C)C)cc21 ; CN(C)CC/C=C1\c2ccccc2CCc2ccc(C(=O)NCCNC(=O)OC(C)(C)C)cc21]

A 25 mL flask, equipped with stir bar, was charged with SL-1826-E (10.6 mg, 22.9 μmol), and the cleavage cocktail (7 mL, 80:20:1 DCM/TFA/TIPS). The resulting light-yellow solution was stirred at 22° C. for 35 minutes at which point HPLC indicated complete consumption of the staring material, and the solvent was removed under reduced pressure. The residue was dissolved in 10 mL MeOH, solvent removed under reduced pressure, and the reaction mixture was purified by preparative HPLC (C18, 5→95% MeCN/H₂O, 0.05% TFA), yielding 6 mg (72% yield) of amine SL-1827 as a clear oil. ¹H NMR (400 MHz, MeOD) δ 7.85 (d, J=2.0 Hz, 1H), 7.67 (dd, J=8.0, 2.0 Hz, 1H), 7.33-7.26 (m, 2H), 7.24 (dt, J=6.6, 3.4 Hz, 1H), 7.22-7.14 (m, 2H), 5.92 (t, J=7.3 Hz, 1H), 3.66 (m, 2H), 3.40 (s, 2H), 3.28-3.20 (m, 2H), 3.17 (t, J=6.0 Hz, 2H), 3.05-2.93 (m, 1H), 2.80 (m, 6H), 2.60 (m, 2H); MS (ESI) calc'd for C₂₃H₃₀N₃O₃⁺ [M+H]⁺ 364.2 found 364.3.

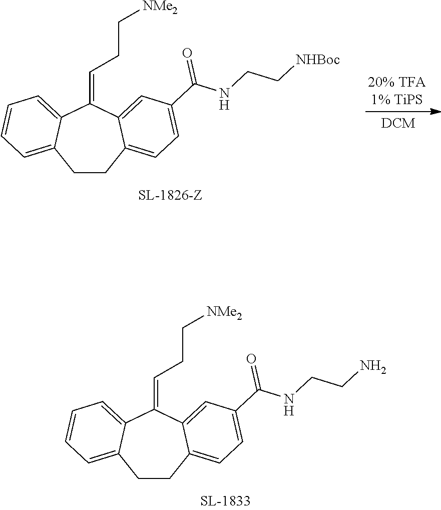 [SMILES: CN(C)CC/C=C1/c2ccccc2CCc2ccc(C(=O)NCCNC(=O)OC(C)(C)C)cc21 ; CN(C)CC/C=C1/c2ccccc2CCc2ccc(C(=O)NCCN)cc21]

A 10 mL flask, equipped with stir bar, was charged with SL-1826-Z (4.7 mg, 10.1 μmol), and the cleavage cocktail (4 mL, 80:20:1 DCM/TFA/TIPS). The resulting light-yellow solution was stirred at 22° C. for 40 minutes at which point HPLC indicated complete consumption of the staring material and the solvent was removed under reduced pressure. The residue was dissolved in 10 mL MeOH, and solvent removed under reduced pressure. The crude residue was used without additional purification in the next step. ¹H NMR (400 MHz, MeOD) δ 7.78 (dd, J=7.9, 1.9 Hz, 1H), 7.68 (d, J=1.9 Hz, 1H), 7.41 (d, J=7.9 Hz, 1H), 7.33-7.22 (m, 1H), 7.21-7.12 (m, 2H), 7.08 (dd, J=7.2, 1.9 Hz, 1H), 5.88 (t, J=7.3 Hz, 1H), 3.66 (t, J=5.9 Hz, 2H), 3.49-3.34 (m, 2H), 3.28-3.20 (m, 2H), 3.16 (t, J=6.0 Hz, 2H), 2.98-2.87 (m, 2H), 2.81 (m, 6H), 2.70-2.49 (m, 2H); MS (ESI) calc'd for C₂₃H₃₀N₃O₃⁺ [M+H]⁺ 364.2 found 364.5.

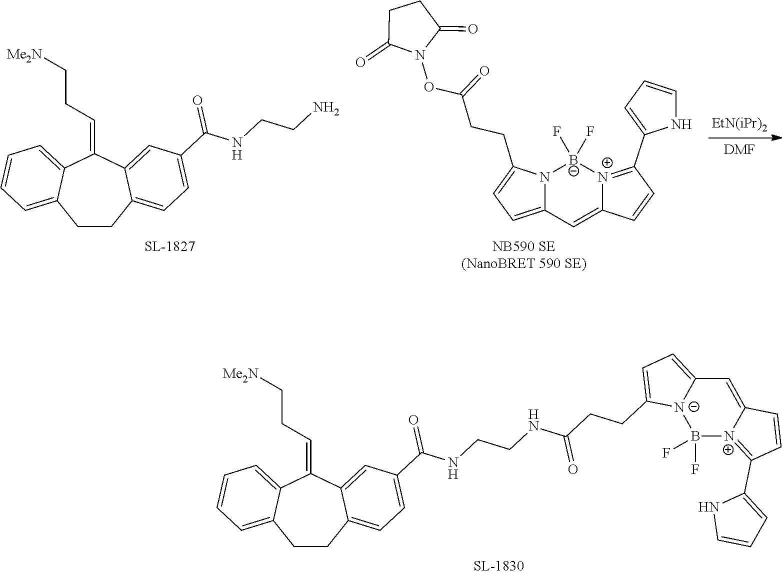

To a solution of SL-1827 (1.3 mg, 3.5 μmol) in DMF (8 mL), DIPEA (3.0 μL, 18 μmol) was added followed by NanoBRET 590 SE (1.5 mg, 3.5 Promega). The resulting solution was allowed to react at 22° C. for 2 hours at which point HPLC analysis indicated full consumption of the starting material. Solvent was removed under vacuum, and the crude residue was purified by preparative RP HPLC (5→95% MeCN/H₂O buffered with 0.5% TFA) to provide 0.9 mg (36% yield) of SL-1830 as a purple film. HPLC: 98% purity at 254 nm; ¹H NMR (400 MHz, MeOD) 1δ 7.71 (d, J=2.0 Hz, 1H), 7.58 (dd, J=8.0, 2.0 Hz, 1H), 7.33-7.23 (m, 2H), 7.23-7.16 (m, 3H), 7.16-7.09 (m, 3H), 7.08 (s, 1H), 7.01 (d, J=4.6 Hz, 1H), 6.72 (d, J=4.0 Hz, 1H), 6.34 (dd, J=3.9, 2.5 Hz, 1H), 6.26 (d, J=4.0 Hz, 1H), 5.82 (t, J=7.3 Hz, 1H), 3.63-3.43 (m, 4H), 3.17-3.05 (m, 2H), 3.01-2.86 (m, 1H), 2.83-2.75 (m, 1H), 2.73-2.69 (m, 8H), 2.56-2.38 (m, 2H); HRMS (SI) Calc'd C₃₉H₄₂BF₂N₆O₂⁺ [M+H]+ 675.3430, found 675.3413.

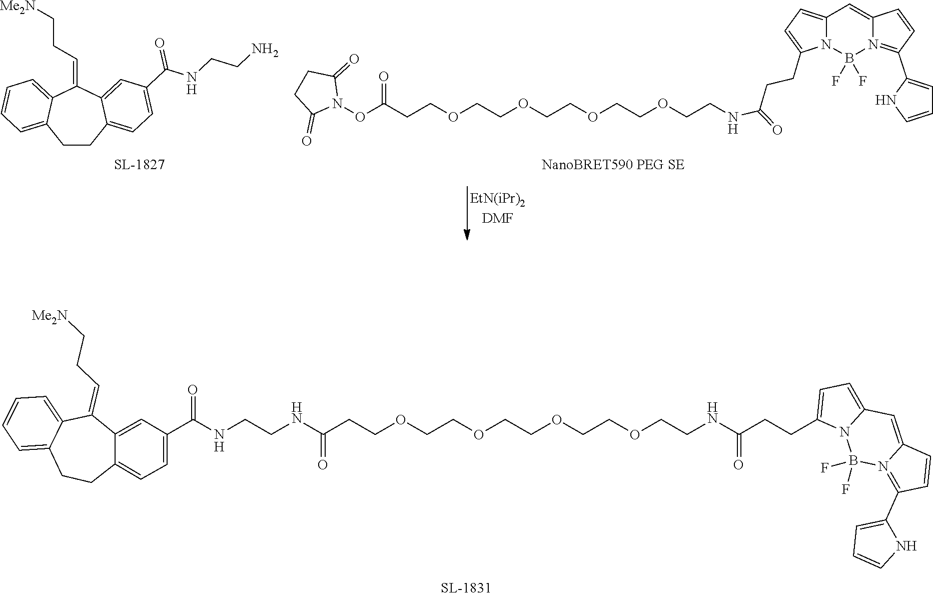

To a solution of SL-1827 (1.6 mg, 4.5 μmol) in DMF (8 mL), DIPEA (6.0 μL, 31 μmol) was added followed by NanoBRET 590 PEG SE (3.0 mg, 4.5 μmol). The resulting solution was allowed to react at 22° C. for 24 hours at which point HPLC analysis indicated full consumption of the starting material. Solvent was removed under vacuum, and the crude residue was purified by preparative RP HPLC (5→95% MeCN/H₂O buffered with 0.5% TFA) to provide 1.1 mg (27% yield) of SL-1831 as a purple film. HPLC: 99% purity at 254 nm; ¹H NMR (400 MHz, MeOD) δ 7.78 (d, J=2.0 Hz, 1H), 7.61 (dd, J=8.0, 2.0 Hz, 1H), 7.33-7.08 (m, 9H), 7.01 (d, J=4.6 Hz, 1H), 6.91 (d, J=4.0 Hz, 1H), 6.40-6.32 (m, 1H), 6.32 (d, J=4.0 Hz, 1H), 5.89 (t, J=7.3 Hz, 1H), 3.66 (t, J=6.0 Hz, 2H), 3.55-3.52 (m, 4H), 3.52-3.40 (m, 12H), 3.37-3.34 (m, 2H), 3.29-3.25 (m, 2H), 3.23-3.15 (m, 2H), 2.82-2.73 (m, 6H), 2.63 (t, J=7.7 Hz, 2H), 2.56 (s, 2H), 2.42 (t, J=6.0 Hz, 2H); HRMS (SI) Calc'd C₅₀H₆₃BF₂N₇O₇⁺ [M+H]+ 922.4850, found 922.4835.

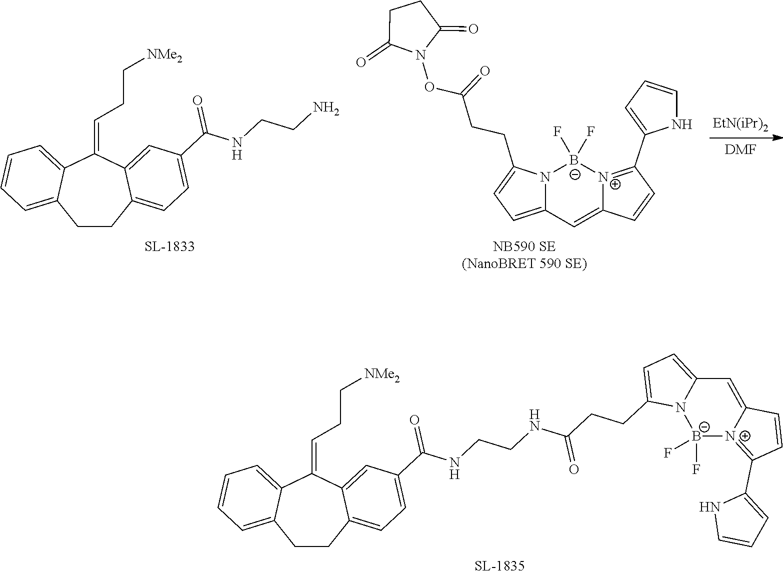

To a solution of SL-1833 (2.1 mg, 5.9 μmol) in DMF (6 mL), DIPEA (5.0 μL, 29 μmol) was added followed by NanoBRET 590 SE (2.5 mg, 5.9 μmol, Promega). The resulting solution was allowed to react at 22° C. for 16 hours at which point HPLC analysis indicated full consumption of the starting material. Solvent was removed under vacuum, and the crude residue was purified by preparative RP HPLC (5→95% MeCN/H₂O buffered with 0.5% TFA) to provide 1.6 mg (41% yield) of SL-1835 as a purple film. HPLC: 99% purity at 254 nm; ¹H NMR (400 MHz, MeOD) δ 7.68 (dd, J=7.9, 2.0 Hz, 1H), 7.58 (d, J=1.9 Hz, 1H), 7.34 (d, J=7.9 Hz, 1H), 7.26 (dd, J=7.5, 1.6 Hz, 1H), 7.23-7.16 (m, 4H), 7.16-7.10 (m, 2H), 7.08 (t, J=7.4 Hz, 1H), 7.02 (d, J=4.6 Hz, 1H), 6.80 (d, J=4.0 Hz, 1H), 6.35 (dd, J=3.9, 2.5 Hz, 1H), 6.26 (d, J=4.0 Hz, 1H), 5.82 (t, J=7.4 Hz, 1H), 3.47-3.42 (m, 2H), 3.35-3.33 (m, 2H), 3.25-3.19 (m, 2H), 2.94-2.81 (m, 2H), 2.77 (s, 6H), 2.65 (t, J=7.7 Hz, 2H), 2.61-2.42 (m, 2H); HRMS (SI) Calc'd C₃₉H₄₂BF₂N₆O₂⁺ [M+H]+ 675.3430, found 675.3429.

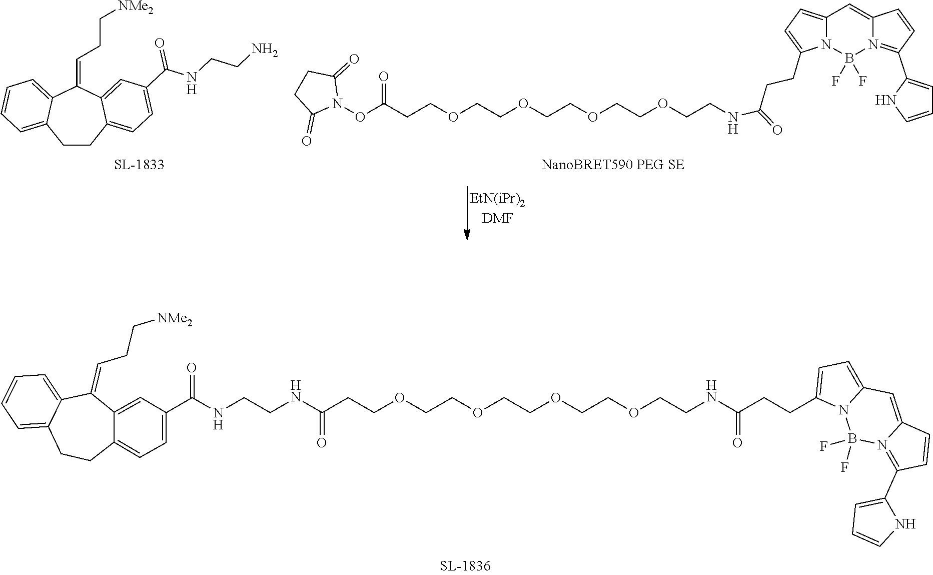

To a solution of SL-1833 (1.0 mg, 3.0 μmol) in DMF (6 mL), DIPEA (4.0 μL, 22 μmol) was added followed by NanoBRET 590-PEG SE (2.0 mg, 3.0 μmol). The resulting solution was allowed to react at 22° C. for 24 hours at which point HPLC analysis indicated full consumption of the starting material. Solvent was removed under vacuum, and the crude residue was purified by preparative RP HPLC (5→95% MeCN/H₂O buffered with 0.5% TFA) to provide 1.5 mg (55% yield) of SL-1836 as a purple film. HPLC: 99% purity at 254 nm; ¹H NMR (400 MHz, MeOD) δ 7.70 (dd, J=7.8, 1.9 Hz, 1H), 7.58 (d, J=1.9 Hz, 1H), 7.36 (d, J=7.9 Hz, 1H), 7.28 (dd, J=7.1, 2.0 Hz, 1H), 7.24-7.10 (m, 6H), 7.07 (dd, J=7.1, 2.0 Hz, 1H), 7.01 (d, J=4.6 Hz, 1H), 6.91 (d, J=3.9 Hz, 1H), 6.35 (dt, J=4.1, 2.4 Hz, 1H), 6.32 (d, J=3.9 Hz, 1H), 5.83 (t, J=7.3 Hz, 1H), 3.63 (t, J=5.9 Hz, 2H), 3.53 (s, 4H), 3.51-3.46 (m, 4H), 3.45-3.36 (m, 10H), 3.36-3.33 (m, 4H), 3.28-3.16 (m, 4H), 2.97-2.84 (m, 2H), 2.81 (s, 6H), 2.63 (t, J=7.7 Hz, 2H), 2.60-2.43 (m, 2H), 2.39 (t, J=6.0 Hz, 2H); HRMS (SI) Calc'd C₅₀H₆₃BF₂N₇O₇⁺ [M+H]+ 922.4850, found 922.4871.

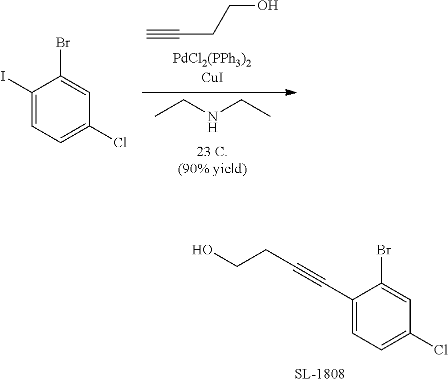

A 50 mL round bottom flask, equipped with stir bar and rubber septum under argon atmosphere, was charged with 2-bromo-4-chloro-1-iodobenzene (2.14 g, 6.74 mmol), CuI (25.7 mg, 0.135 mmol), and PdCl₂(PPh₃)₂ (95 mg, 0.13 mmol). Degassed diethylamine (12 mL) was added via syringe followed by 3-butyn-1-ol. The reaction mixture was stirred at 23° C. for 48 hours at which point HPLC indicated complete consumption of the staring material, and the solvent removed under reduced pressure. The crude residue was purified by flash chromatography (gradient elution, 0→40% EtOAc/heptane, yielding 1.57 g (90% yield) of alkyne SL-1808 as a yellow solid. ¹H NMR (400 MHz, CDCl3) δ 7.59 (d, J=2.1 Hz, 1H), 7.37 (d, J=8.3 Hz, 1H), 7.23 (dd, J=8.4, 2.1 Hz, 1H), 3.85 (t, J=6.1 Hz, 2H), 2.74 (t, J=6.1 Hz, 2H); ¹³C NMR (100 MHz, CDCl3) δ 134.3, 133.7, 132.1, 127.5, 126.0, 124.0, 92.7, 80.5, 60.9, 24.0; HRMS (ESI) calc'd for C₁₀H₉BrClO [M+H]⁺ 258.9525 found 258.9521.

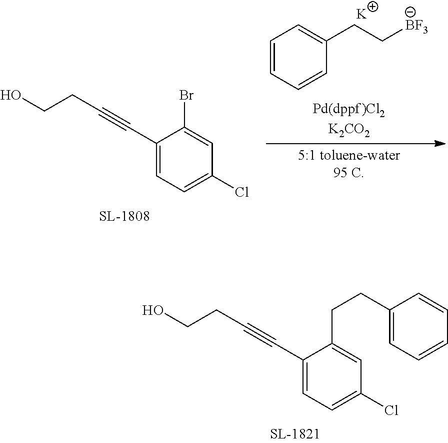

A 500 mL round bottom flask, equipped with stir bar and reflux condenser was charged with SL-1808 (1.57 g, 6.05 mmol), potassium trifluoro(phenethyl)borate (1.35 g, 6.35 mmol), K₂CO₃ (2.22 g, 18.2 mmol), and Pd(dppf)Cl₂ (220 mg, 0.30 mmol). Flask was evacuated and backfilled with argon (3× times repeat). Degassed toluene (50 mL) and H₂O (10 mL) were added, and the reaction mixture was stirred at 95° C. for 24 hours. The reaction mixture was cooled to ambient temperature, and solvents were removed under reduces pressure. The crude residue was partitioned between DCM (100 mL) and water (100 mL), aqueous layer was extracted to DCM (2×100 mL), organics were combined, dried over MgSO₄, filtered, and the solvent removed under reduced pressure. The crude residue was initially purified by flash chromatography (gradient elution, 0→100% EtOAc/heptane and then further purified by preparative HPLC (C18, 5→95% MeCN/H₂O, 0.05% TFA) yielding 392 mg (23% yield) of alkyne SL-1821 as a white solid. ¹H NMR (400 MHz, CDCl3) δ 7.38-7.27 (m, 3H), 7.24-7.16 (m, 3H), 7.16-7.08 (m, 2H), 3.82 (t, J=6.3 Hz, 2H), 3.10-2.97 (m, 2H), 2.96-2.81 (m, 2H), 2.74 (t, J=6.3 Hz, 2H); ¹³C NMR (100 MHz, CDCl3) δ 1 13C NMR (101 MHz, CDCl3) δ 145.4, 141.4, 133.7, 133.5, 128.9, 128.4, 128.4, 126.2, 126.1, 121.3, 90.9, 80.0, 61.2, 36.7, 36.7, 24.0; HRMS (ESI) calc'd for C₁₈H₁₈ClO [M+H]⁺ 285.1046 found 285.1044.

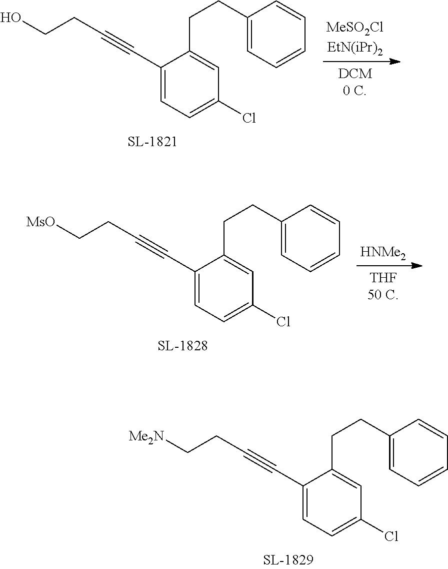 [SMILES: OCCC#Cc1ccc(Cl)cc1CCc1ccccc1 ; CS(=O)(=O)OCCC#Cc1ccc(Cl)cc1CCc1ccccc1 ; CN(C)CCC#Cc1ccc(Cl)cc1CCc1ccccc1 ; "HNMe2"]

A 50 mL round bottom flask, equipped with stir bar, was charged with SL-1821 (210 mg, 0.74 mmol), EtN(iPr)₂ (263 μL, 1.47 mmol), and DCM (20 mL). The resulting solution was cooled to 0° C. under argon followed by addition of mesyl chloride (86 μL, 1.1 mmol). The reaction mixture was stirred at 0° C. for 4 hours at which point HPLC indicated complete consumption of the staring material. The reaction was quenched by addition of saturated aqueous K₂CO₃ (20 mL), and aqueous phase was further extracted with DCM (3×20 mL). Organics were combined, dried over MgSO4, filtered, and the solvent removed under reduced pressure. The crude residue was used without additional purification in the next step. ¹H NMR (400 MHz, CDCl3) δ 7.37-7.27 (m, 3H), 7.25-7.07 (m, 5H), 4.37 (t, J=6.8 Hz, 2H), 3.12-2.99 (m, 2H), 2.98 (s, 3H), 2.96-2.84 (m, 4H).

A 50 mL round bottom flask, equipped with stir bar, was charged with SL-1828 (270 mg, 0.74 mmol) and 2M dietylamine solution in THF (37 mL, 74 mmol). The reaction mixture was stirred at 50° C. for 17 hours at which point HPLC indicated complete consumption of the staring material, and the solvent removed under reduced pressure. The crude residue was purified by flash chromatography (gradient elution, 0→20% MeOH/DCM, yielding 125 mg (54% yield) of amine SL-1829 as a clear oil. ¹H NMR (400 MHz, CDCl3) δ 7.39-7.27 (m, 3H), 7.24-7.18 (m, 3H), 7.16-7.03 (m, 2H), 3.08-2.95 (m, 2H), 2.95-2.83 (m, 2H), 2.63 (s, 4H), 2.31 (s, 6H); ¹³C NMR (100 MHz, CDCl3) δ 145.4, 141.6, 133.4, 128.8, 128.4, 126.1, 126.0, 121.7, 58.4, 45.1, 36.7, 36.7, 18.5; HRMS (ESI) calc'd for C₂₀H₂₃ClN [M+H]⁺ 312.1519 found 312.1516.

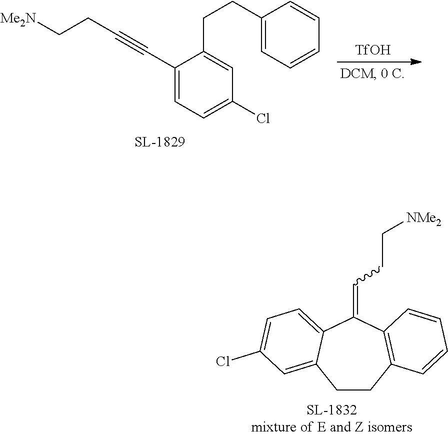

A 50 mL round bottom flask, equipped with stir bar, was charged with a solution of SL-1829 (120 mg, 0.38 mmol) in DCM (15 mL). The solution was cooled to 0° C., and triflic acid (170 μL, 1.9 mmol) was added in one portion. The resulting brown solution is stirred at 0° C. for 10 minutes at which point the reaction was quenched by addition of saturated aqueous solution of K₂CO₃ (15 mL). Organic layer was separated, and the aqueous solution was extracted (2× 15 mL DCM). Organics were combined, dried over MgSO4, filtered, and concentrated in vacuo. The crude residue was purified by flash chromatography (gradient elution, 0→30% MeOH/DCM, yielding 101 mg (99% yield) of amine SL-1832 as a clear oil. ¹H NMR (400 MHz, CDCl3, reported for mixture of E/Z isomers) δ 7.27-7.23 (m, 1H), 7.23-6.98 (m, 6H), 5.86 (m, 1H), 3.65-3.12 (m, 2H), 2.94 (s, 1H), 2.75 (s, 1H), 2.50-2.34 (m, 2H), 2.29 (m, 2H), 2.19 (s, 6H); ¹³C NMR (100 MHz, CDCl3, reported for mixture of E/Z isomers) δ 142.6, 142.5, 141.2, 140.8, 139.7, 139.6, 139.0, 138.8, 138.5, 136.7, 132.8, 132.5, 130.0, 129.9, 129.8, 129.6, 129.6, 128.6, 128.1, 128.0, 127.6, 127.2, 126.1, 126.0, 125.9, 125.8, 59.2, 45.2, 45.2, 33.7, 33.4, 31.9, 31.7, 27.8, 27.7. HRMS (ESI) calc'd for C₁₀H₂₃ClN [M+H]⁺ 312.1519 found 312.1510. Single peak on HPLC at 254 nm.

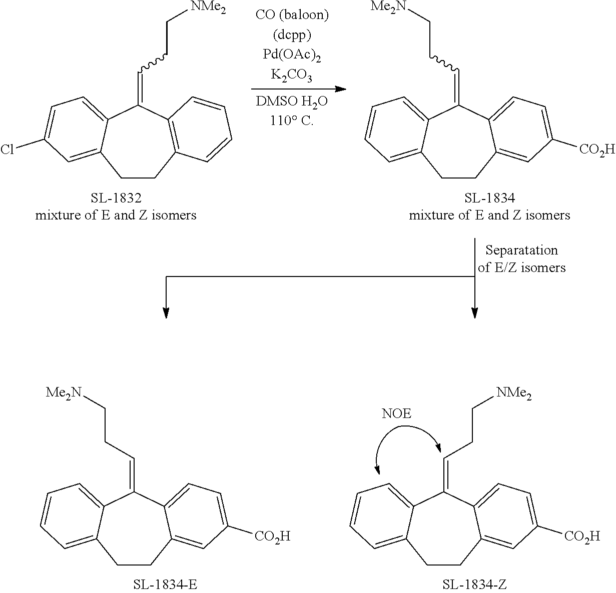 [SMILES: CN(C)CCC=C1c2ccccc2CCc2cc(Cl)ccc21 ; CN(C)CCC=C1c2ccccc2CCc2cc(C(=O)O)ccc21 ; CN(C)CC/C=C1\c2ccccc2CCc2cc(C(=O)O)ccc21 ; CN(C)CC/C=C1/c2ccccc2CCc2cc(C(=O)O)ccc21]

A 50 mL round bottom flask, equipped with stir bar and septum, was charged with SL-1832 (100 mg, 0.32 mmol), K₂CO₃ (130 mg, 0.96 mmol), Pd(OAc)₂ (7.2 mg, 32 μmol), and [dcpp 2BF₄] (39 mg, 64 μmol). Flask was evacuated and backfilled with argon (3× times repeated). Degassed DMSO (8 mL) and H₂O (0.8 mL) were added, and reaction vessel evacuated and backfilled with carbon monoxide (3× times repeat). CO was allowed to bubble through the solution for 5 minutes. The resulting yellow suspension was heated to 110° C. under CO balloon for 22 hours at which point HPLC analysis indicated complete consumption of the starting material. The reaction mixture was diluted with MeOH (8 mL), passed through syringe filter, and purified by preparative HPLC (C18, 5→95% MeCN/H₂O, 0.05% TFA) yielding 54 mg (51% yield) of SL-1834-E as a clear oil and 51 mg (48% yield) of SL-1834-Z as a clear oil. E isomer has shorter retention time than Z isomer.

Characterization data for SL-1834-E: ¹H NMR (400 MHz, MeOD) δ 7.82 (dd, J=8.0, 1.8 Hz, 1H), 7.78 (d, J=1.8 Hz, 1H), 7.43 (d, J=8.0 Hz, 1H), 7.36-7.24 (m, 3H), 7.23-7.16 (m, 1H), 5.93 (t, J=7.3 Hz, 1H), 3.39 (t, J=9.0 Hz, 2H), 3.26 (q, J=7.4 Hz, 2H), 3.02 (d, J=14.9 Hz, 1H), 2.82 (d, J=8.1 Hz, 7H), 2.60 (dd, J=16.9, 8.3 Hz, 2H); ¹³C NMR (100 MHz, MeOD) δ 169.6, 147.7, 146.1, 140.6, 139.7, 138.7, 132.8, 131.1, 129.7, 129.6, 129.6, 129.1, 128.5, 127.4, 126.5, 58.0, 34.8, 32.7, 26.2; HRMS (ESI) calc'd for C₂₁H₂₄NO₂ [M+H]⁺ 322.1807 found 322.1807.

Characterization data for SL-1834-Z: ¹H NMR (400 MHz, MeOD) δ 7.97 (d, J=1.7 Hz, 1H), 7.92 (dd, J=7.8, 1.8 Hz, 1H), 7.37-7.25 (m, 2H), 7.25-7.13 (m, 2H), 7.10 (dd, J=7.2, 1.9 Hz, 1H), 5.89 (t, J=7.3 Hz, 1H), 3.55-3.36 (m, 2H), 3.26 (q, J=8.6 Hz, 2H), 2.97 (d, J=25.6 Hz, 2H), 2.82 (d, J=6.3 Hz, 6H), 2.68-2.45 (m, 2H); ¹³C NMR (100 MHz, MeOD) δ 169.5, 147.6, 145.4, 141.2, 140.8, 138.2, 131.7, 131.3, 130.7, 129.5, 129.1, 129.0, 128.8, 127.4, 125.9, 58.0, 34.5, 32.8, 26.2; HRMS (ESI) calc'd for C₂₁H₂₄NO₂ [M+H]⁺ 322.1807 found 322.1807.

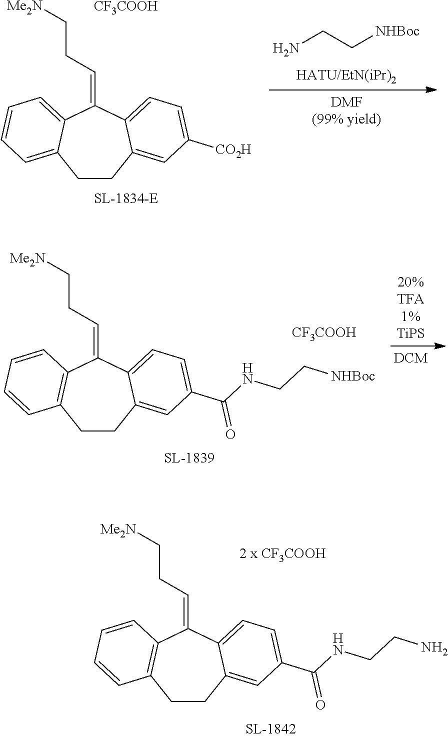

A 25 mL flask, equipped with stir bar, was charged with SL-1834-E TFA salt (36 mg, 83 μmol), tert-butyl (2-aminoethyl) carbamate (39 mg, 103 μmol), HATU (17 mg, 0.10 mmol), EtN(iPr)₂ (74 μL, 0.41 mmol), and DMF (8 mL). The resulting light-yellow solution was stirred at 22° C. for 18 hours at which point HPLC indicated complete consumption of the staring material, and the solvent was removed under reduced pressure. The crude residue was purified by preparative HPLC (C18, 5→95% MeCN/H₂O, 0.05% TFA), yielding 47 mg (99% yield) of amide SL-1829 as a clear oil. MS (ESI) calc'd for C₂₈H₃₈NO₃ [M+H]⁺ 464.3 found 464.4. Single peak on HPLC at 254 nm.

A 10 mL flask, equipped with stir bar, was charged with SL-1839 (20 mg, 35 μmol), and the cleavage cocktail (4 mL, 80:20:1 DCM/TFA/TIPS). The resulting light-yellow solution was stirred at 22° C. for 75 minutes at which point HPLC indicated complete consumption of the staring material, and the solvent was removed under reduced pressure. The residue was dissolved in 10 mL MeOH, and solvent removed under reduced pressure. The crude residue was used without additional purification in the next step. MS (ESI) calc'd for C₂₃H₃₀N₃O₃⁺ [M+H]⁺ 364.2 found 364.3. Single peak on HPLC at 254 nm.

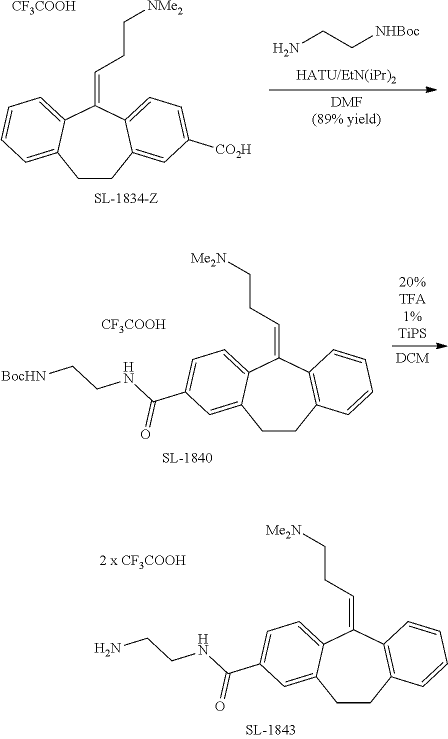

A 25 mL flask, equipped with stir bar, was charged with SL-1834-Z TFA salt (26 mg, 81 μmol), tert-butyl (2-aminoethyl) carbamate (39 mg, 0.10 mmol), HATU (16 mg, 0.10 mmol), EtN(iPr)₂ (72 μL, 0.40 mmol), and DMF (8 mL). The resulting light-yellow solution was stirred at 22° C. for 17 hours at which point HPLC indicated complete consumption of the staring material, and the solvent was removed under reduced pressure. The crude residue was purified by preparative HPLC (C18, 5→95% MeCN/H2O, 0.05% TFA), yielding 33 mg (89% yield) of amide SL-1840 as a clear oil. MS (ESI) calc'd for C₂₈H₃₈NO₃ [M+H]⁺ 464.3 found 464.4. Single peak on HPLC at 254 nm.

A 10 mL flask, equipped with stir bar, was charged with SL-1840 (15 mg, 35 μmol), and the cleavage cocktail (4 mL, 80:20:1 DCM/TFA/TIPS). The resulting light-yellow solution was stirred at 22° C. for 85 minutes at which point HPLC indicated complete consumption of the staring material, and the solvent removed under reduced pressure. The residue was dissolved in 10 mL MeOH, and solvent removed under reduced pressure. The crude residue was used without additional purification in the next step. MS (ESI) calc'd for C₂₃H₃₀N₃O₃⁺ [M+H]⁺ 364.2 found 364.4; Single peak on HPLC at 254 nm.

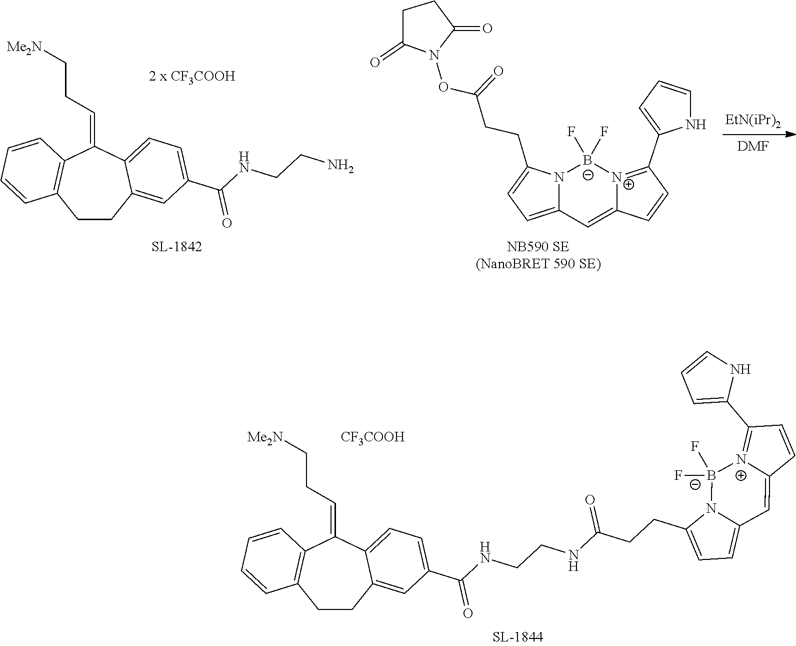

To a solution of SL-1842 TFA salt (4 mg, 7 μmol) in DMF (6 mL), DIPEA (9.0 μL, 49 μmol) was added followed by NanoBRET 590 SE (3.0 mg, 7.0 μmol, Promega). The resulting solution was allowed to react at 22° C. for 17 hours at which point HPLC analysis indicated full consumption of the starting material. Solvent was removed under vacuum, and the crude residue was purified by preparative RP HPLC (5→95% MeCN/H₂O buffered with 0.5% TFA) to provide 3.6 mg (76% yield) of SL-1844 as a purple film. HPLC: 99% purity at 254 nm; ¹H NMR (400 MHz, MeOD) δ 7.55 (dd, J=8.1, 1.9 Hz, 1H), 7.50 (d, J=1.9 Hz, 1H), 7.35 (d, J=8.1 Hz, 1H), 7.30-7.18 (m, 3H), 7.22-7.12 (m, 4H), 7.10 (s, 1H), 7.00 (d, J=4.5 Hz, 1H), 6.78 (d, J=4.0 Hz, 1H), 6.35 (dt, J=4.0, 2.3 Hz, 1H), 6.27 (d, J=4.0 Hz, 1H), 5.83 (t, J=7.3 Hz, 1H), 3.51-3.40 (m, 4H), 3.37-3.34 (m, 2H), 3.29-3.24 (m, 2H), 3.23-3.15 (m, 2H), 3.02-2.88 (m, 2H), 2.84-2.72 (m, 7H), 2.65 (t, J=7.7 Hz, 2H), 2.59-2.46 (m, 2H); HRMS (SI) Calc'd C₃₉H₄₂BF₂N₆O₂⁺ [M+H]+ 675.3430, found 675.3426.

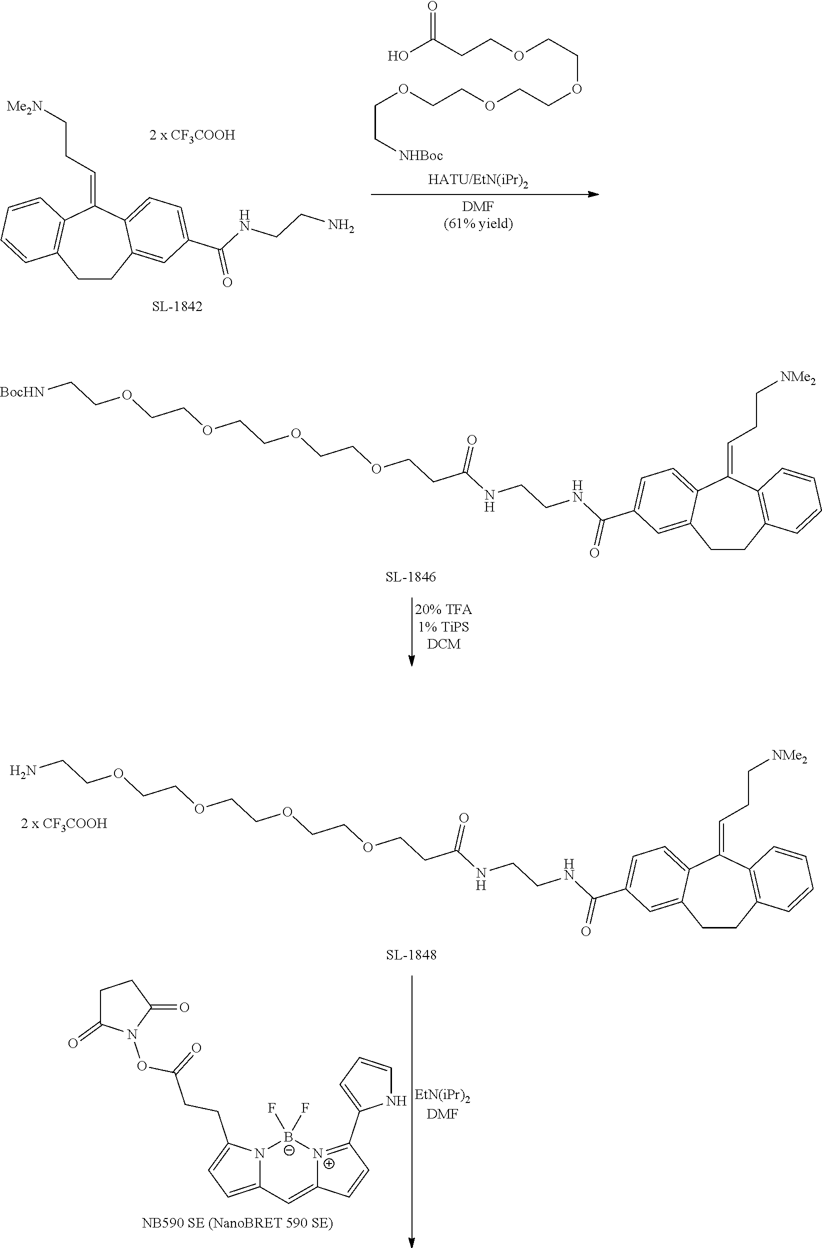 [SMILES: CN(C)CC/C=C1\c2ccccc2CCc2cc(C(=O)NCCN)ccc21 ; CC(C)(C)OC(=O)NCCOCCOCCOCCOCCC(=O)O ; CN(C)CC/C=C1\c2ccccc2CCc2cc(C(=O)NCCNC(=O)CCOCCOCCOCCOCCNC(=O)OC(C)(C)C)ccc21 ; CN(C)CC/C=C1\c2ccccc2CCc2cc(C(=O)NCCNC(=O)CCOCCOCCOCCOCCN)ccc21 ; O=C(CCc1ccc2n1[B-](F)(F)[N+]1=C(c3ccc[nH]3)C=CC1=C2)ON1C(=O)CCC1=O]

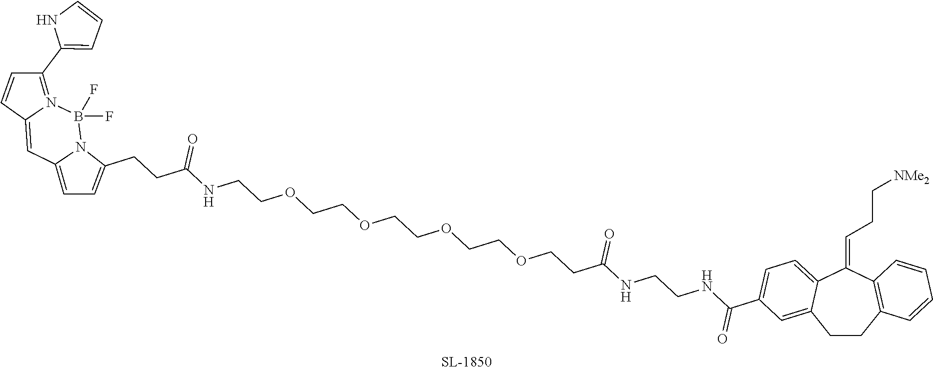

A 25 mL flask, equipped with stir bar, was charged with SL-1842 (5.2 mg, 8.8 μmol), 2,2-dimethyl-4-oxo-3,8,11,14,17-pentaoxa-5-azaicosan-20-oic acid (4.0 mg, 11 μmol), HATU (4.2 mg, 11 μmol), EtN(iPr)₂ (11 μL, 62 μmol), and DMF (6 mL). The resulting light-yellow solution was stirred at 22° C. for 17 hours at which point, HPLC indicated complete consumption of the staring material and the solvent was removed under reduced pressure. The crude residue was purified by preparative HPLC (C18, 5→95% MeCN/H2O, 0.05% TFA), yielding 3.8 mg (61% yield) of amide SL-1846 as a clear oil. 1H NMR (400 MHz, MeOD) δ 7.64 (dd, J=8.0, 1.9 Hz, 1H), 7.59 (d, J=1.9 Hz, 1H), 7.42 (d, J=8.0 Hz, 1H), 7.37-7.23 (m, 3H), 7.20 (dd, J=6.8, 1.6 Hz, 1H), 5.92 (t, J=7.3 Hz, 1H), 3.71 (t, J=6.0 Hz, 2H), 3.59-3.37 (m, 20H), 3.29-3.13 (m, 4H), 3.03 (q, J=15.7, 14.5 Hz, 1H), 2.82 (m, 7H), 2.61 (t, J=9.2 Hz, 2H), 2.45 (t, J=6.0 Hz, 2H), 1.44 (s, 9H); HRMS (ESI) calc'd for C₃₉H₅₉N₄O₈ [M+H]⁺ 711.4333 found 711.4329. Single peak on HPLC at 254 nm.

A 10 mL flask, equipped with stir bar, was charged with SL-1846 (3.8 mg, 4.6 μmol), and the cleavage cocktail (4 mL, 80:20:1 DCM/TFA/TIPS). The resulting light-yellow solution was stirred at 22° C. for 60 minutes at which point HPLC indicated complete consumption of the staring material, and the solvent removed under reduced pressure. The residue was dissolved in 10 mL MeOH, and solvent removed under reduced pressure. The crude residue was used without additional purification in the next step. MS (ESI) calc'd for C₃₄H₅₂N₄O₆²⁺ [M+H]²⁺/2 306.2 found 306.4; Single peak on HPLC at 254 nm.

To a solution of SL-1848 (4 mg, 5 μmol) in DMF (6 mL) was added DIPEA (6.0 μL, 33 μmol) followed by NanoBRET 590 SE (2.0 mg, 4.7 μmol, Promega). The resulting solution was allowed to react at 22° C. for 23 hours at which point HPLC analysis indicated full consumption of the starting material. Solvent was removed under vacuum, and the crude residue was purified by preparative RP HPLC (5→95% MeCN/H₂O buffered with 0.5% TFA) to provide 3.0 mg (70% yield) of SL-1850 as a purple film. HPLC: 99% purity at 254 nm; ¹H NMR (400 MHz, MeOD) δ 7.60 (dd, J=8.1, 1.9 Hz, 1H), 7.55 (d, J=1.9 Hz, 1H), 7.37 (d, J=8.0 Hz, 1H), 7.28 (td, J=4.5, 4.1, 2.7 Hz, 2H), 7.26-7.17 (m, 5H), 7.17-7.10 (m, 1H), 7.01 (d, J=4.6 Hz, 1H), 6.91 (d, J=4.0 Hz, 1H), 6.34 (t, J=3.1 Hz, 1H), 6.31 (d, J=4.0 Hz, 1H), 5.86 (t, J=7.3 Hz, 1H), 3.65 (t, J=6.0 Hz, 2H), 3.56-3.43 (m, 16H), 3.41 (d, J=5.6 Hz, 2H), 3.35 (m, 4H), 3.27 (d, J=7.7 Hz, 2H), 3.19 (s, 2H), 2.97 (s, 1H), 2.77 (d, J=13.9 Hz, 7H), 2.63 (t, J=7.7 Hz, 2H), 2.55 (t, J=8.8 Hz, 2H), 2.40 (t, J=6.0 Hz, 2H); HRMS (SI) Calc'd C₅₀H₆₃BF₂N₇O₇⁺ [M+H]+ 922.4850, found 922.4847.

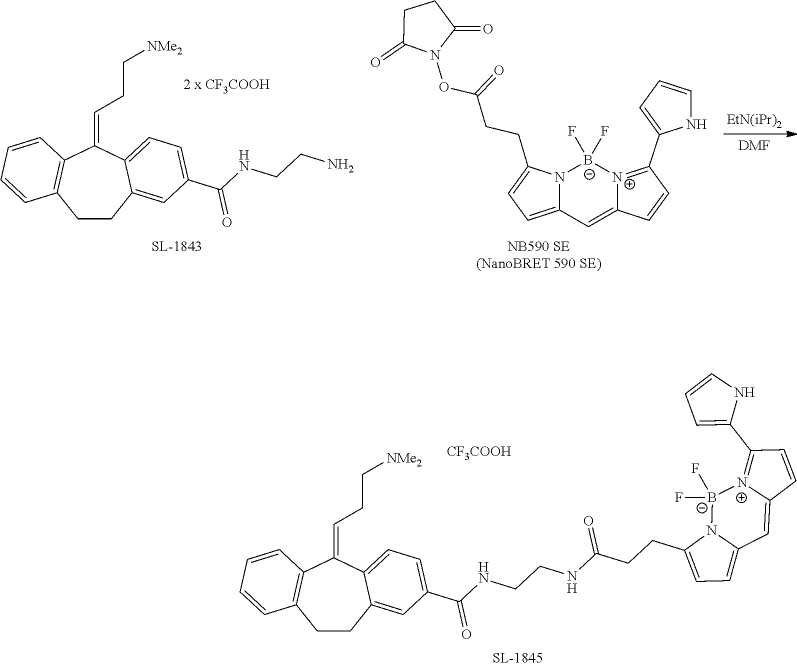

To a solution of SL-1843 TFA salt (4 mg, 7 μmol) in DMF (6 mL), DIPEA (9.0 μL, 49 μmol) was added followed by NanoBRET 590 SE (3.0 mg, 7.0 Promega). The resulting solution was allowed to react at 22° C. for 17 hours at which point HPLC analysis indicated full consumption of the starting material. Solvent was removed under vacuum, and the crude residue was purified by preparative RP HPLC (5→95% MeCN/H₂O buffered with 0.5% TFA) to provide 3.6 mg (76% yield) of SL-1844 as a purple film. HPLC: 99% purity at 254 nm; ¹H NMR (400 MHz, MeOD) δ 10.76 (s, 1H), 7.74 (d, J=1.9 Hz, 1H), 7.60 (dd, J=7.9, 1.8 Hz, 1H), 7.37-7.27 (m, 1H), 7.21 (tq, J=5.5, 2.7, 2.3 Hz, 5H), 7.16 (d, J=4.6 Hz, 1H), 7.12 (s, 1H), 7.10-7.05 (m, 1H), 7.03 (d, J=4.6 Hz, 1H), 6.82 (d, J=3.9 Hz, 1H), 6.37 (dt, J=4.2, 2.4 Hz, 1H), 6.31 (d, J=4.0 Hz, 1H), 5.83 (t, J=7.3 Hz, 1H), 3.52 (m, 4H), 3.16 (t, J=7.8 Hz, 2H), 2.94 (d, J=23.2 Hz, 2H), 2.82-2.60 (m, 8H), 2.49 (t, J=8.8 Hz, 2H); HRMS (SI) Calc'd C₃₉H₄₂BF₂N₆O₂⁺ [M+H]+ 675.3430, found 675.3421.

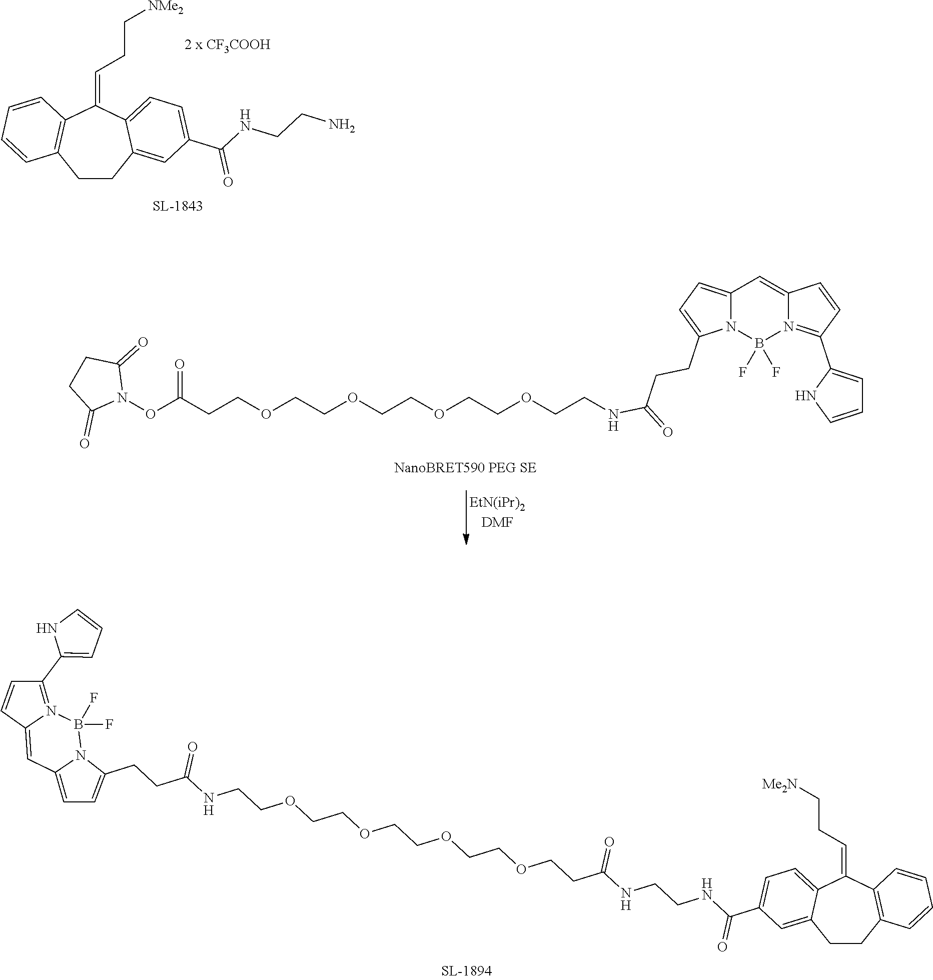

To a solution of SL-1843 (8.0 mg, 14 μmol) in DMF (6 mL), DIPEA (12 μL, 68 μmol) was added followed by NanoBRET 590-PEG SE (5.5 mg, 8.1 μmol, Promega). The resulting solution was allowed to react at 22° C. for 2 hours at which point HPLC analysis indicated full consumption of the starting material. Solvent was removed under vacuum, and the crude residue was purified by preparative RP HPLC (5→95% MeCN/H₂O buffered with 0.5% TFA) to provide 2.3 mg (19% yield) of SL-1894 as a purple film. HPLC: 99% purity at 254 nm; ¹H NMR (400 MHz, MeOD) δ 7.74 (d, J=1.9 Hz, 1H), 7.68 (dd, J=7.8, 1.9 Hz, 1H), 7.38-7.11 (m, 8H), 7.07 (dd, J=7.2, 1.9 Hz, 1H), 7.01 (d, J=4.5 Hz, 1H), 6.91 (d, J=4.0 Hz, 1H), 6.35 (dt, J=4.0, 2.3 Hz, 1H), 6.32 (d, J=4.0 Hz, 1H), 5.83 (t, J=7.3 Hz, 1H), 3.66 (t, J=6.0 Hz, 2H), 3.53 (s, 4H), 3.50-3.45 (m, 10H), 3.45-3.38 (m, 2H), 3.35 (d, J=5.4 Hz, 2H), 3.26 (d, J=7.7 Hz, 2H), 3.23-3.14 (m, 2H), 2.97-2.87 (m, 2H), 2.78 (d, J=14.9 Hz, 6H), 2.63 (t, J=7.7 Hz, 2H), 2.53 (t, J=14.3 Hz, 2H), 2.41 (t, J=6.0 Hz, 2H); HRMS (SI) Calc'd C₅₀H₆₃BF₂N₇O₇⁺ [M+H]+ 922.4850, found 922.4859.

SEQUENCES
WT OgLuc
(SEQ ID NO: 1)
MFTLADFVGDWQQTAGYNQDQVLEQGGLSSLFQALGVSVTPIQKVVLS
GENGLKADIHVIIPYEGLSGFQMGLIEMIFKVVYPVDDHHFKIILHYG
TLVIDGVTPNMIDYFGRPYPGIAVFDGKQITVTGTLWNGNKIYDERLI
NPDGSLLFRVTINGVTGWRLCENILA
WT OgLuc Lg
(SEQ ID NO: 2)
MFTLADFVGDWQQTAGYNQDQVLEQGGLSSLFQALGVSVTPIQKVVLS
GENGLKADIHVIIPYEGLSGFQMGLIEMIFKVVYPVDDHHFKIILHYG
TLVIDGVTPNMIDYFGRPYPGIAVFDGKQITVTGTLWNGNKIYDERLI
NPD
WT OgLuc β9
(SEQ ID NO: 3)
GSLLFRVTIN
WT OgLuc β10
(SEQ ID NO: 4)
GVTGWRLCENILA
NanoLuc
(SEQ ID NO: 5)
MVFTLEDFVGDWRQTAGYNLDQVLEQGGVSSLFQNLGVSVTPIQRIVL
SGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERL
INPDGSLLFRVTINGVTGWRLCERILA
NanoLuc Lg
(SEQ ID NO: 6)
MVFTLEDFVGDWRQTAGYNLDQVLEQGGVSSLFQNLGVSVTPIQRIVL
SGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERL
INPD
NanoLuc β9
(SEQ ID NO: 7)
GSLLFRVTINV
NanoLuc β10
(SEQ ID NO: 8)
GVTGWRLCERILA
LgBiT
(SEQ ID NO: 9)
MVFTLEDFVGDWRQTAGYNLDQVLEQGGVSSLFQNLGVSVTPIQRIVL
SGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERL
INPDGSLLFRVTIN
SmBiT
(SEQ ID NO: 10)
VTGYRLFEEIL
HiBiT
(SEQ ID NO: 11)
VSGWRLFKKIS
LgTrip (3546)
(SEQ ID NO: 12)
MKHHHHHHVFTLDDFVGDWEQTAAYNLDQVLEQGGVSSLLQNLAVSVT
PIMRIVRSGENALKIDIHVIIPYEGLSADQMAQIEEVFKVVYPVDDHH
FKVILPYGTLVIDGVTPNKLNYFGRPYEGIAVFDGKKITTTGTLWNGN
KIIDERLITPD
SmTrip9
(SEQ ID NO: 13)
GSMLFRVTINS
β9/β10 dipeptide
(SEQ ID NO: 14)
GSMLFRVTINSVSGWRLFKKIS
His5
(SEQ ID NO: 15)
HHHHH
HisX6
(SEQ ID NO: 16)
HHHHHH
C-myc
(SEQ ID NO: 17)
EQKLISEEDL
Flag
(SEQ ID NO: 18)
DYKDDDDK
SteptTag
(SEQ ID NO: 19)
WSHPQFEK
HA Tag
(SEQ ID NO: 20)
YPYDVPDYA

1. A composition comprising a broad-spectrum G-protein coupled receptor (GPCR) binding agent attached to a functional element or solid surface, wherein the broad-spectrum GPCR binding agent comprises: 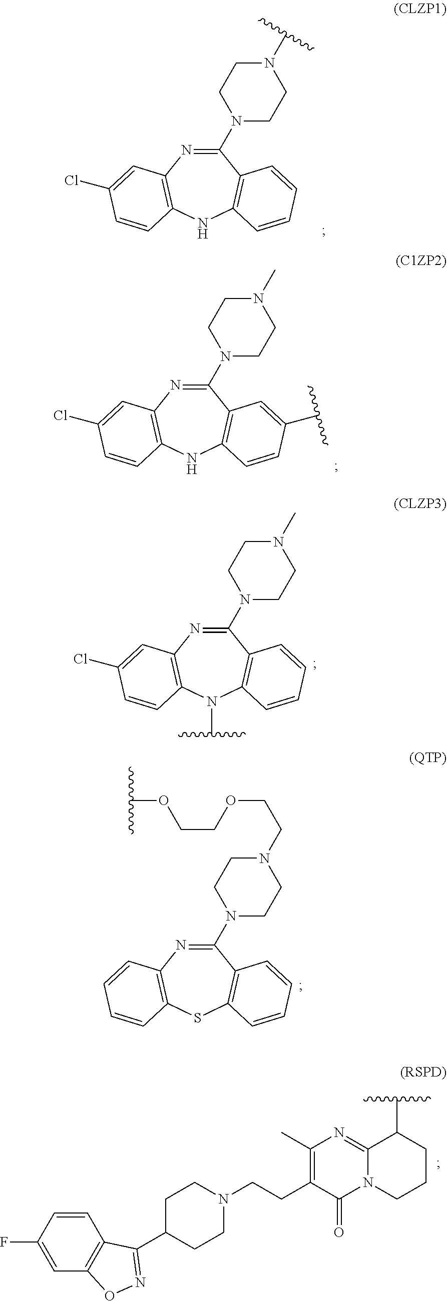 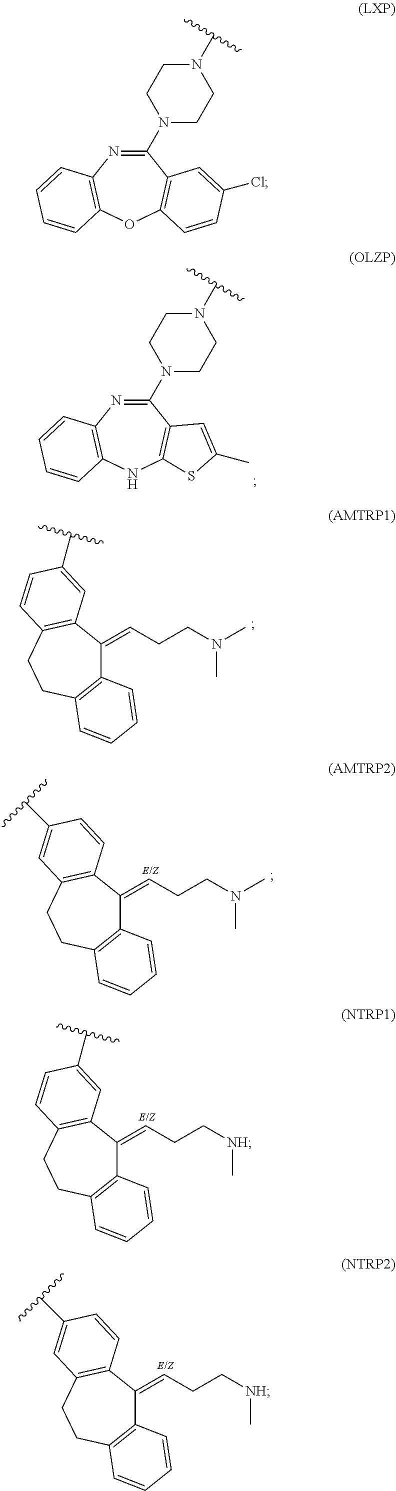
wherein [symbol] is the point of attachment of the broad-spectrum GPCR binding agent to the functional element, solid surface, or a linker between the broad-spectrum GPCR binding agent and the functional element or solid surface, and wherein the broad-spectrum GPCR binding agent may exist as the cis isomer (Z), trans isomer (E), or a mixture of the two.

2-11. (canceled)

12. The composition of claim 1, wherein the solid surface is selected from a sedimental particle, a membrane, glass, a tube, a well, a self-assembled monolayer, a surface plasmon resonance chip, or a solid support with an electron conducting surface.

13. The composition of claim 12, wherein the sedimental particle is a magnetic particle.

14. The composition of claim 1, wherein the functional element is selected from a detectable element, an affinity element, and a capture element.

15. The composition of claim 14, wherein the detectable element comprises a fluorophore, chromophore, radionuclide, electron opaque molecule, a MM contrast agent, SPECT contrast agent, or mass tag.

16. The composition of claim 1, wherein the broad-spectrum GPCR binding agent is attached to the functional element or solid surface directly.

17. The composition of claim 1, wherein the broad-spectrum GPCR binding agent is attached to the functional element or solid surface via a linker.

18. The composition of claim 17, wherein the linker comprises [(CH₂)₂O]ₙ, wherein n is 1-20.

19. The composition of claim 17, wherein the linker is attached to the broad-spectrum GPCR binding agent and/or the functional element by an amide bond.

20. The composition of claim 1, comprising a structure of: 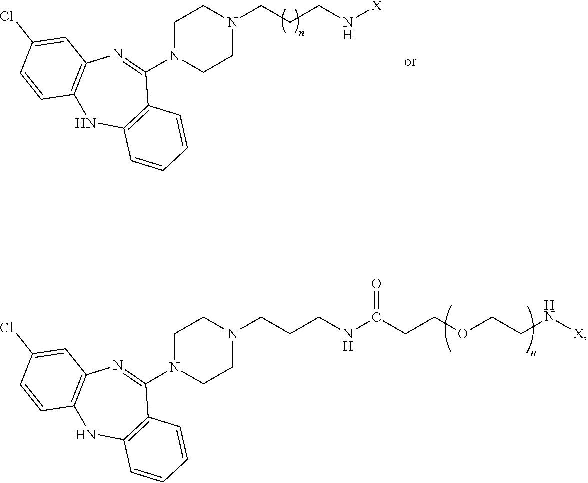
wherein n is 0-8, and wherein X is a functional element or solid surface 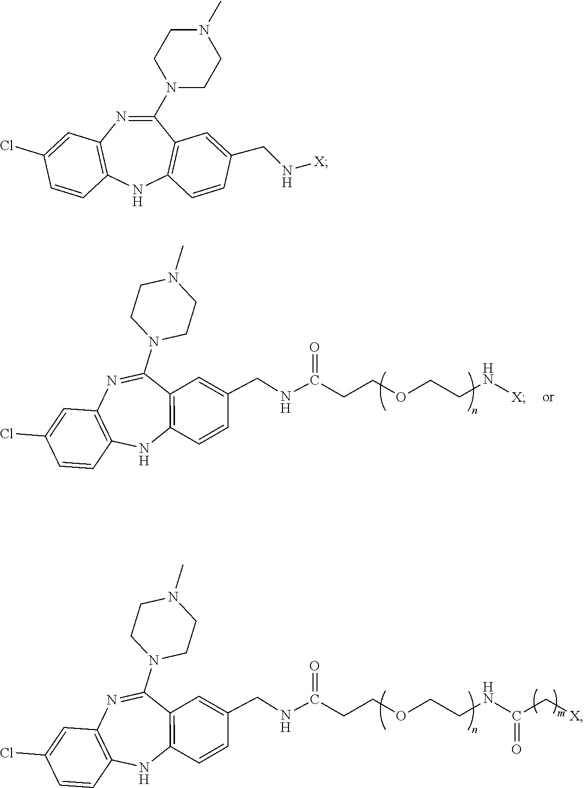
wherein n is 0-8, wherein m is 0-8, and wherein X is a functional element or solid surface; 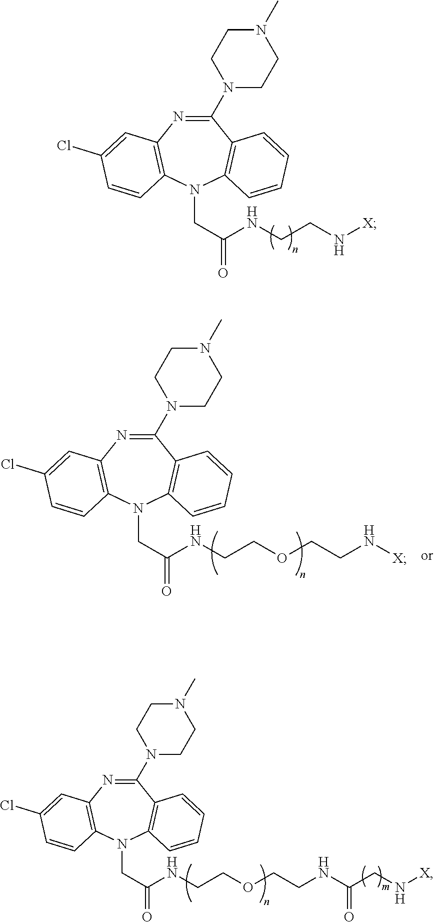
wherein n is 0-8, wherein m is 0-8, and wherein X is a functional element or solid surface; 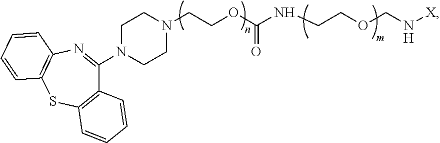
wherein n is 0-8, wherein m is 0-8, and wherein X is a functional element or solid surface; 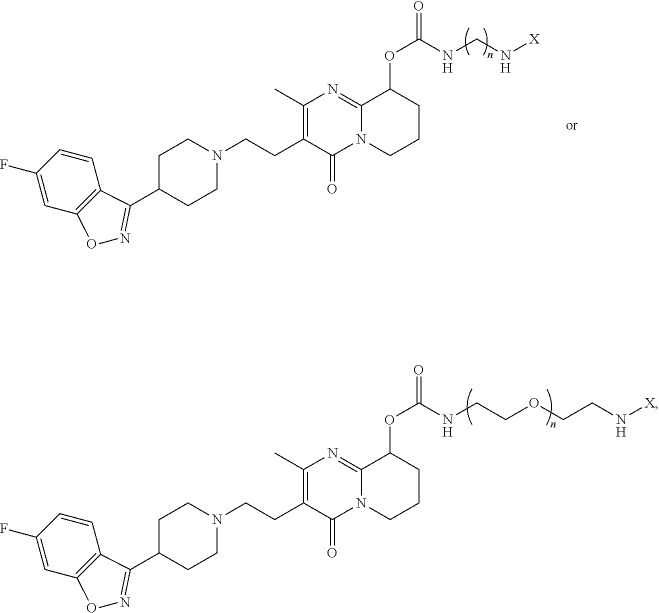
wherein n is 0-8, and wherein X is a functional element or solid surface; 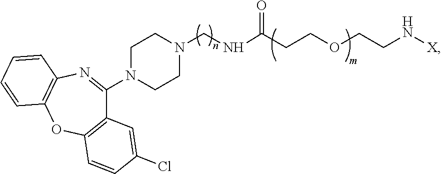
wherein n is 0-8, wherein m is 0-8, and wherein X is a functional element or solid surface; 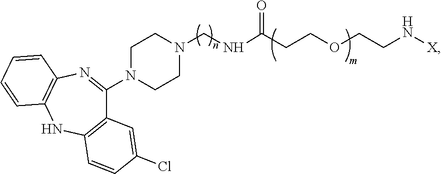
wherein n is 0-8, wherein m is 0-8, and wherein X is a functional element or solid surface; 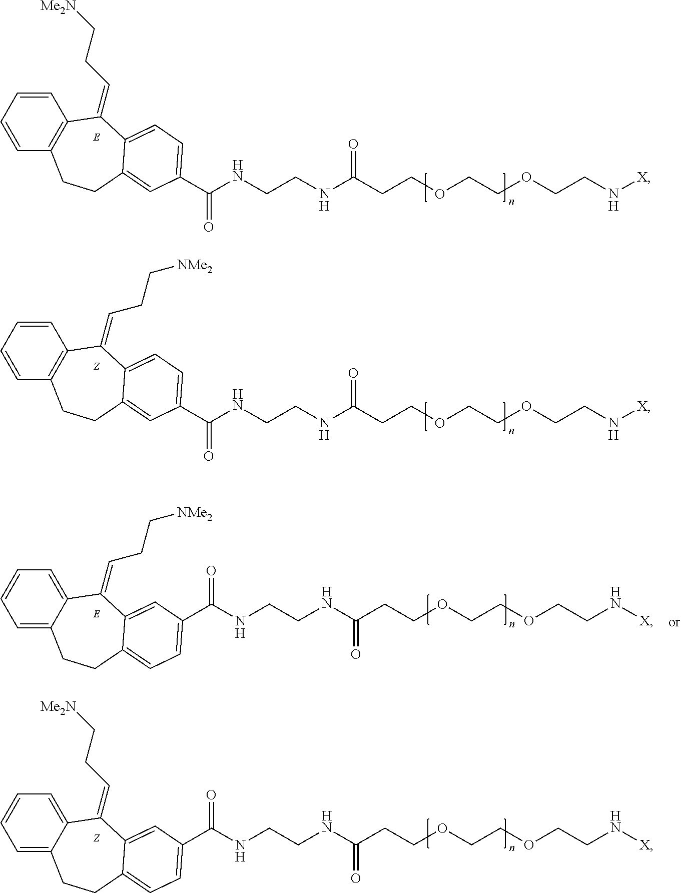
wherein n is 0-8 and wherein X is a functional element or solid surface; 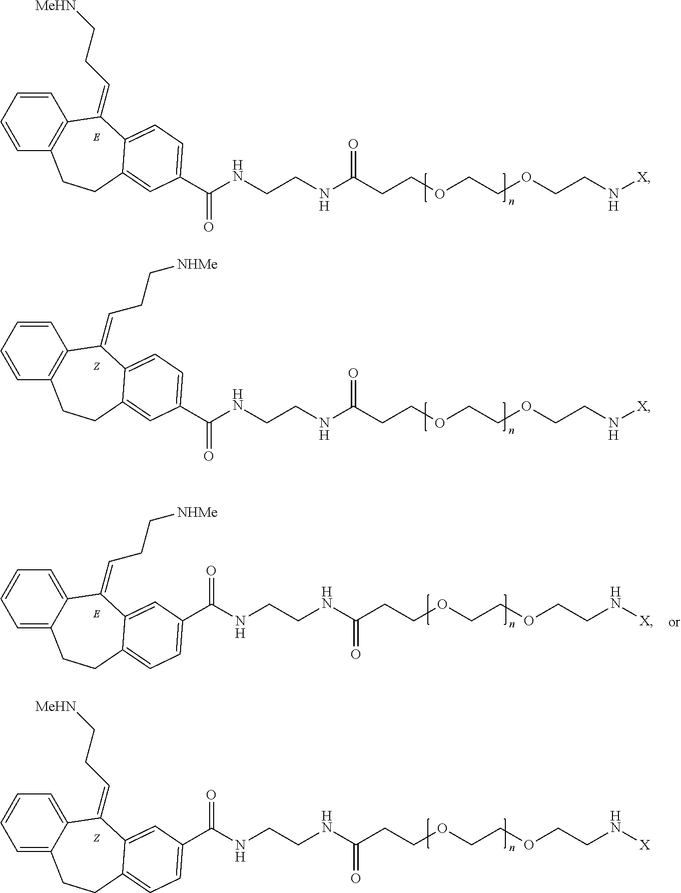
wherein n is 0-8 and wherein X is a functional element or solid surface; 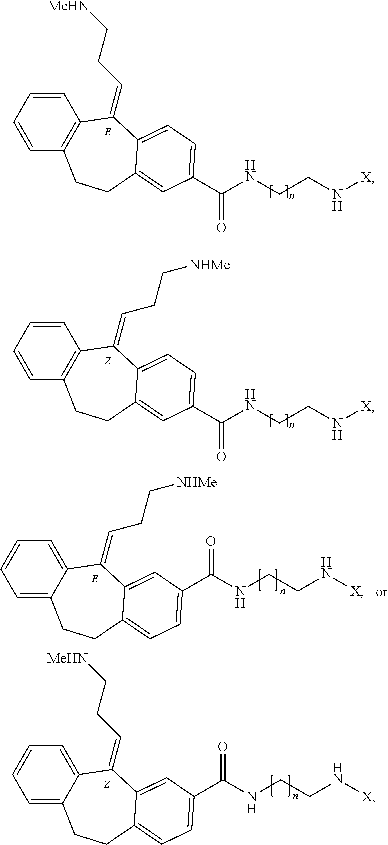
wherein n is 0-8 and wherein X is a functional element or solid surface; or 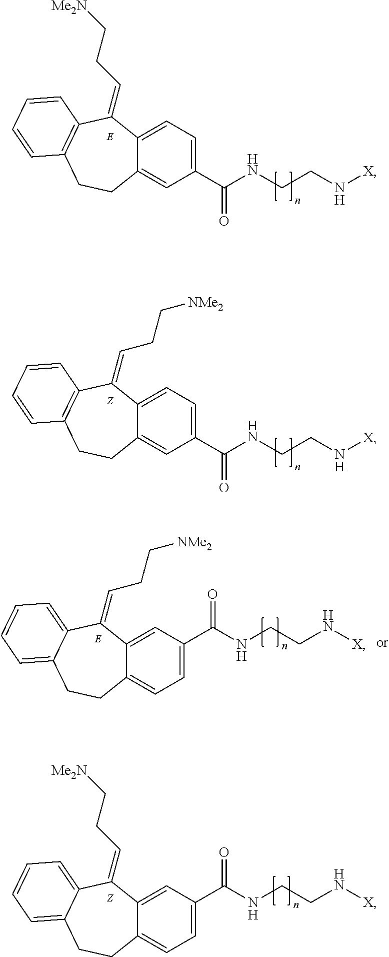
wherein n is 0-8 and wherein X is a functional element or solid surface.

21-30. (canceled)

31. The composition of claim 20, wherein X is a fluorophore.

32. The composition of claim 1, comprising a non-natural abundance of one or more stable heavy isotopes.

33. A method of detecting or quantifying GPCRs in a sample, comprising contacting the sample with a composition of claim 1 and detecting or quantifying the functional element of a signal produced thereby.

34. The method of claim 33, wherein the functional element of a signal produced thereby is detected or quantified by fluorescence, mass spectrometry, optical imaging, magnetic resonance imaging (MM), and energy transfer.

35. A method of isolating GPCRs from a sample, comprising contacting the sample with a composition of claim 1 and separating the functional element or the solid surface, as well as the bound GPCRs, from the unbound portion of the sample.

36. A method of characterizing the identities of the GPCRs in a sample comprising isolating the GPCRs from a sample by the method of claim 35, and analyzing the isolated GPCRs by mass spectrometry.

37. A method of monitoring interactions between GPCRs and unmodified biomolecules comprising contacting the sample with a composition of any claim 1.

38. The method of claim 33, wherein the sample is selected from a cell, cell lysate, body fluid, tissue, biological sample, in vitro sample, and environmental sample.

39. A system comprising:
(a) composition of claim 1, wherein the functional element is a fluorophore; and
(b) a fusion of a GPCR and a bioluminescent protein or a peptide component of a bioluminescent complex, wherein the emission spectrum of the bioluminescent protein or the bioluminescent complex overlaps the excitation spectrum of the fluorophore.

40. The system of claim 39, comprising a kit, cell, cell lysate, or reaction mixture.

41. The system of claim 39, wherein the fusion comprises a GPCR and a peptide component of a bioluminescent complex, and wherein the system further comprises one or more additional components of the bioluminescent complex and a substrate for the bioluminescent complex.

42. A method comprising:
(a) contacting a fusion of a GPCR and a bioluminescent protein, with
(i) a composition of claim 1 wherein the functional element is a fluorophore and wherein the emission spectrum of the bioluminescent protein overlaps the excitation spectrum of the fluorophore, and
(ii) a substrate for the bioluminescent protein; and
(b) detecting a wavelength of light within the excitation spectrum of the fluorophore resulting from bioluminescence resonance energy transfer from the bioluminescent protein to the fluorophore when the broad-spectrum GPCR binding agent is bound to the GPCR.

43. A method comprising:
(a) contacting a fusion of a GPCR and a peptide component of a bioluminescent complex, with
(i) a composition of claim 1 wherein the functional element is a fluorophore and wherein the emission spectrum of the bioluminescent protein overlaps the excitation spectrum of the fluorophore,
(ii) a polypeptide component of the bioluminescent complex. and
(iii) a substrate for the bioluminescent protein; and
(b) detecting a wavelength of light within the excitation spectrum of the fluorophore resulting from bioluminescence resonance energy transfer from the bioluminescent complex to the fluorophore when the broad-spectrum GPCR binding agent is bound to the GPCR.
